(19) United States
(12) Patent Application Publication
(10) Pub. No.: US 2023/0053473 A1
(43) Pub. Date: Feb. 23, 2023

(54) ENHANCING EXPRESSION OF LINE-1 ENCODED ORF2P FOR CANCER THERAPEUTICS

(71) Applicant: The Johns Hopkins University, Baltimore, MD (US)

(72) Inventor: Kathleen H. Burns, Baltimore, MD (US)

(21) Appl. No.: 17/789,099

(22) PCT Filed: Dec. 26, 2020
(86) PCT No.: PCT/US2020/067060

§ 371 Date: Jun. 24, 2022

Related U.S. Application Data
(60) Provisional application No. 62/953,743, filed on Dec. 26, 2019.

(51) Int. Cl. A61K 31/69; A61K 31/52; A61K 31/428; A61K 31/4184; A61K 38/06; A61K 31/4015; A61K 38/05; A61K 38/07; A61K 31/4439; A61K 31/24; A61K 45/06; A61P 35/00

(52) U.S. Cl. CPC A61K 31/69; A61K 31/52; A61K 31/428; A61K 31/4184; A61K 38/06; A61K 31/4015; A61K 38/05; A61K 38/07; A61K 31/4439; A61K 31/24; A61K 45/06; A61P 35/00

(57) ABSTRACT

Methods for treating a neoplasia are provided. Further provided are novel targets for cancer chemotherapy including ORF2p Protein and methods for increasing expression of ORF2p in neoplasias. Monoclonal antibodies are also provided to various epitopes of ORF2p and their use in research and diagnostivds.

### CROSS-REFERENCE TO RELATED APPLICATION

The present application claims the benefit of U.S. provisional application No. 62/953,743, filed Dec. 26, 2019, which is incorporated by reference herein in its entirety.

### BACKGROUND

Mobile elements make up nearly half of the human genome. The most prevalent sequences are retrotransposons, which propagate via RNA intermediates, and of these, modern activity resides with the Long INterspersed Element-1 (LINE-1, L1) sequences and those elements mobilized by L1 proteins in trans. L1 is the only autonomous (protein-coding), functional retrotransposon in humans, and each of us inherits a distinct complement of active elements. Mobilization occurs after a retrotransposition-competent L1 is transcribed, translated into proteins encoded by its open reading frames (ORFs), ORF1p and ORF2p, and packaged into a ribonucleoprotein (RNP) complex. ORF2p encodes an endonuclease that cuts the genomic DNA target site and a reverse transcriptase that generates L1 cDNA.

Many malignant tissues undergo L1 promoter hypomethylation and permit L1 expression and somatic retrotransposition. Both targeted and genome-wide sequencing efforts have identified thousands of de novo insertions that have occurred across hundreds of human cancers. Several groups have shown that L1 ORF1p expression is a hallmark of many different cancers. Of these many cancer types, it has been shown that L1 upregulation is induced early in the development of ovarian cancers, where ORF1p accumulation is evident within precursor lesions of the fallopian tube. L1 retrotransposition can also contribute directly to cellular transformation; in colon cancers, acquired L1 insertions are known to cause driving mutations in the adenomatous polyposis coli (APC) tumor suppressor.

Many studies have focused on host factors that alter retrotransposition efficiency or on the functional effects of acquired LINE-1 insertions; fewer have focused on cellular effects of LINE-1 expression. LINE-1 is known to be toxic, but the mechanisms underlying its toxicity are unclear. ORF2p appears to incite DNA double-strand breaks (DSBs) in some systems, although it is thought to function as a single-strand nickase in retrotransposition. Despite its toxicity, LINE-1 promoter hypomethylation and ORF1p protein expression are hallmarks of human cancers and retrotransposition is commonplace in these diseases. This paradox reflects a lack of understanding surrounding LINE-1 toxicity and how malignant cells tolerate LINE-1 expression.

ORF2p is strictly required for retrotransposition. Whether the protein can be directly detected has been a matter of some debate. In experimental systems, ORF2p is translated from the bicistronic transcript through an unconventional mechanism. Compared to ORF1p, ectopically expressed ORF2p accumulates in substoichiometric amounts (ORF1p:ORF2p ratio >30:1) and may be restricted to a subset of cells within a population. Reports of endogenously expressed ORF2p have been more limited than ORF1p.

### SUMMARY

In accordance with an embodiment, the present invention provides a method for treatment of a neoplasia in a cell or population of cells comprising increasing expression of ORF2p in the cell or population of cells.

In accordance with an embodiment, the present invention provides a method for treatment of a neoplasia in a cell or population of cells comprising increasing expression of ORF2p in the cell or population of cells by targeting (e.g., inhibition of) protein kinase RNA activated (PKR) in the cell or population of cells.

In accordance with an embodiment, the present invention provides a method for treatment of a neoplasia in a cell or population of cells comprising increasing expression of ORF2p in the cell or population of cells by targeting (e.g., inhibition of) protein kinase RNA activated (PKR) in the cell or population of cells in combination with one or more additional chemotherapeutic or biological agents.

In accordance with an embodiment, the present invention provides a method for treatment of a neoplasia in a subject in need thereof, comprising administering to the subject a biologically active agent which increases expression of ORF2p in the neoplasia of the subject.

In accordance with an embodiment, the present invention provides a method for treatment of a neoplasia in a subject in need thereof, comprising administering to the subject a biologically active agent which increases expression of ORF2p in the neoplasia of the subject in combination with one or more additional chemotherapeutic agents.

In accordance with an embodiment, the present invention provides a method for treatment of a neoplasia in a subject in need thereof, comprising increasing expression of ORF2p in the neoplasia of the subject by targeting (e.g. inhibition) protein kinase RNA activated (PKR) in the neoplasia of the subject.

In accordance with an embodiment, the present invention provides a method for treatment of a neoplasia in a subject in need thereof, comprising increasing expression of ORF2p in the neoplasia of the subject by targeting (e.g. inhibition) protein kinase RNA activated (PKR) in the neoplasia of the subject in combination with one or more additional chemotherapeutic or biologic agents.

In preferred aspects, cells of a patient, or a targeted neoplasia sample, or a tissue sample of a patient suffering from a neoplasia will be assessed for ORF1p and/or ORF2p levels.

In particular preferred aspects, cells of a patient, or a targeted neoplasia sample, or a tissue sample of a patient suffering from a neoplasia will be assessed for reduced ORF2p expression as compared to ORF1p expression. For instance, a tissue sample may be assessed for ORF2p expression by use of an antibody disclosed herein. A sample or patient assessed as having lower ORF2p levels as compared to ORF1p (e.g. 20, 25, 30, 40, 50, 60, 70, 80, 90, 100 percent less or multiple (e.g. 2, 3, 4, 5, 6, 7, 8, 9, 10) fold less ORF2p levels relative to ORF1p would be identified and selected for treatment, particularly for instance to target PKR or other regulators of ORF2p translational or to inhibit proteasomal degradation of ORF2p. Similarly, the ratio of ORF2p to ORF1p will be less than 1 in a targeted sample such as tumor biposy. Patients may be identified and selected for treatment based on ORF2p levels, particularly low ORF2p expression. For instance, a tissue sample may be assessed for ORF2p expression by use of an antibody disclosed herein.

In many instances, the differential expression of ORF2p and ORF1p will be substantial. For instance, ORF2p expression may be detected at very low levels or even undetected in a targeted neoplasia sample using an antibody-based assay such as a Western blot.

In accordance with an embodiment, the present invention provides monoclonal antibodies that specifically bind ORF2p antigenic epitopes. In some embodiments, the ORF2p antigenic epitope is selected from the group consisting of DRSTRQ (SEQ ID NO: 1), LHQADLID (SEQ ID NO: 2), KASRRQEITKIRAE (SEQ ID NO: 3), KELEKQEQT (SEQ ID NO: 4), QDIGVGKD (SEQ ID NO: 5). Such antibodies can be used for assessing levels of expression of ORF2p in a cell or population of cells or tissue sample.

In accordance with an embodiment, the present invention provides the use of a monoclonal antibody that specifically binds ORF2p antigenic epitopes to detect expression of ORF2p in a cell or population of cells comprising administration of one or more of said monoclonal antibodies to the cell or population of cells.

In some embodiments, the ORF2p antigenic epitope is selected from the group consisting of DRSTRQ (SEQ ID NO: 1), LHQADLID (SEQ ID NO: 2), KASRRQEITKIRAE (SEQ ID NO: 3), KELEKQEQT (SEQ ID NO: 4), QDIGVGKD (SEQ ID NO: 5).

The present methods and systems also may utilize antibodies to assess expression of ORF1p in a cell or population of cells or tissue sample, including relative to ORF2p expression levels as disclosed herein. In some embodiments, the ORF1p antigenic epitope may be MENDFDELRE (recognized by 4H1).

Kits are also provided which may comprise 1) antibodies for assessing ORF2p expression levels and 2) antibodies for assessing ORF1p expression levels.

A variety of types of cancers may be treated and identified and selected for treatment with the present methods and systems, including without limitation breast cancer, colon cancer, esophageal cancer, gastric cancer, head and neck cancer, lung cancer, melanoma, ovarian cancer, pancreatic cancer, and prostate cancer.

Other aspects of the invention are disclosed infra.

### BRIEF DESCRIPTION OF THE DRAWINGS

FIGS. 1A-1D. Heterogeneous LINE-1 expression in colon cancer. 1A) ORF1p immunohistochemistry stain of formalin-fixed paraffin-embedded (FFPE) colon cancer tissue. LINE-1 immunostaining is seen in tumor (T) and not in normal colonic epithelium (N). The arrow indicates a transition from normal to tumor within a gland. Scale bar=50 μm. 1B) Immunohistochemistry stain of FFPE colon cancer tissue from patient case 191. Left, low magnification of ORF1p intensely-positive and negative tumor sectors. Right, low magnification of CDX2, a colon epithelium marker. LINE-1(+) cells express higher CDX2 and are gland-forming whereas LINE-1(−) cells express lower CDX2 and do not form glands. Scale bars=500 μm. 1C) Phylogenetic tree of the tumor subclones in case 191 based on TIP-seq and known tumor driver alleles. The number of de novo LINE insertions is indicated along the line edges (red). We genotyped by Sanger sequencing known tumor driver alleles and found an AKT1E17K mutation in the CDX2-dim cells and a TP53R248Q mutation in CDX2-high cells (both primary and metastatic sites). All tumor specimens possessed a BRAFV600E allele regardless of LINE-1 expression status. The color of the lines indicates the presence or absence of known tumor driver alleles. 1D) Ki67 quantification of normal epithelium, LINE-1(+) glandular cancer, and LINE-1(−) solid cancer in case 191. The percent of positive cells was calculated as the number of Ki67+ nuclei divided by the total number of epithelial cell nuclei. Three independent high-powered fields were counted per tissue morphology, and results were compared with ANOVA and two-sided T tests. Scale bar=100 μm.

FIGS. 2A-2G. LINE-1 inhibits cell growth in RPE by activating the p53-p21 pathway. 2A) LINE-1 sequence. The 5′ untranslated region (UTR) is a CpG-rich RNA polymerase II promoter. Open reading frame (ORF) 1 and ORF2 are separated by a 63 bp linker sequence. ORF2 has endonuclease (EN, red) and reverse transcriptase (RT, gray) domains. 2B) Above, episomal pCEP4 mammalian expression vector for eGFP (pDA083) or LINE-1 (pDA077). AbxR=antibiotic selection marker, EBNA1=Epstein-Barr Nuclear Antigen 1, oriP=EBNA-1 replication origin. Below, western blot of ORF1p and ORF2p from RPE cells transfected with each plasmid. Uncropped blot is shown in the Source Data. 2C) Clonogenic assay (day 12). Cells are transfected with eGFP (pDA083) or LINE-1 (pDA077). Representative plates with number of colonies indicated ±SD. Quantification to the right is normalized to eGFP-expressing cells set at 100%, with n=3 independent experiments. P value calculated by two-sided unpaired T test. 2D) Clonogenic assay (day 12). Cells are treated with lentivirus encoding TP53 shRNA (+) or control vector (−). Data presented as the rate of LINE-1 per 100 eGFP colonies ±SEM, n=3 independent experiments. P value obtained by unpaired two-sided T test. 2E) Positive Selection CRISPR-Cas9 knockout screen workflow using the Brunello CRISPR knockout library. RPE-Cas9=RPE cells constitutively expressing Cas9 protein. KO=knockout. sgRNA=single-guide RNA. NGS=Next-Generation Sequencing. NTC=Non-targeting-control. 2F) Screen enrichment rank vs. significance values of gene knockouts that rescue growth of LINE-1(+) cells. The red line is the FWER-adjusted genome-wide significance level. Low ranks indicate rescue of LINE-1(+) cells. 2G) CRISPR knockout of TP53 or CDKN1A significantly rescue growth of RPE compared to non-targeting-control (NTC). Representative plates with all data presented as LINE-1/100 eGFP colonies ±SEM. n=2 biological replicates. P value obtained by unpaired one-sided T test.

FIGS. 3A-3E. LINE-1 activates a p53 and IFN response. 3A) Left: Volcano plot of differentially expressed genes. Vertical dashed lines indicate fold-change of −1 or 1 (loge) and the horizontal dashed line indicates a FWER-controlled p-value of 0.05. Right: histograms of gene set enrichment analysis results. Gene set names are indicated above each plot. The number of genes is indicated on the y-axis and the x-axis indicates differential expression bins. Individual genes comprising these datasets are highlighted in the volcano plot according to the colors of the bars in the histograms. Data derived from n=3 independent replicates. 3B) Violin plots illustrating differential expression of p53 transcriptional targets. Direct and indirect target genes are curated from published reports (Fischer et al., 2016 and Fischer, 2017). Horizontal bars mark median values. The number of genes in each group are indicated below the plot. 3C) Histogram of gene set enrichment results of interferon (IFN) signaling genes. The number of genes is indicated on the y-axis and the x-axis indicates differential expression. 3D) Relative fold-change of interferon B1 (IFNB1) and A1 (IFNA1) in LINE-1(+) compared to luciferase(+) cells measured by RNAseq. Error bars indicate SEM. 3E) RNAseq analysis revealed upregulation of the RNA sensing pathway involving Toll-like receptor 3 (TLR3), RIG-I (DDX58), and MDAS (IFIH1) in LINE-1(+) cells. Error bars indicate SEM.

FIGS. 4A-4D. Mapping LINE-1 fitness interactions in TP53-deficient cells. 4A) TP53^{KD} cells are RPE-Cas9 cells stably transduced with shRNA to knockdown p53 and then engineered to express luciferase (pDA094) or codon optimized LINE-1 (pDA095) in a doxycycline-inducible manner (Tet-On). Tet-On cells were transduced with the Brunello CRISPR KO library at a multiplicity of infection of 0.3 and puromycin-selected for 8 days before inducing expression of LINE-1 or luciferase for 27 days. Cell pools were sampled at 4-5 day intervals and analyzed for sgRNA representation with MAGeCK. Count data are normalized to reads that align to 1,000 built-in non-targeting-control (NTC) sgRNAs (black). NGS=Next Generation Sequencing. KO=Knockout. 4B) Genes shown as rank ordered plot of Stauffer Z scores (Zₛ) with a family-wise error rate (FWER) of 0.05. Inset indicates the number of 95% confidence interval overlaps over all time points between LINE-1 and luciferase groups among gene knockouts that meet the FWER threshold (red) versus those that do not (gray). 4C) Heatmap of 1,390 significant genes depicting the Z scores over time, ranked by Zₛ. There are 1,366 synthetic lethal interactions and 24 rescue interactions. Most knockouts achieved detectable effects by 17-22 days into the screen, evidenced by increasing gene Z scores during these time points. 4D) Overlap of genes with LINE-1 fitness interactions observed in the present study with genes previously known to interact with LINE-1 proteins physically or by modifying retrotransposition. Previously known LINE-1 interactors were identified by Liu et al., 2018, Moldovan et al., 2015, Taylor et al., 2013, and Goodier et al., 2013.

FIGS. 5A-5H. The Fanconi Anemia (FA) pathway is essential in p53-deficient cells. 5A) Network plot of 75 DNA repair genes identified in the screen. Edges indicate known physical interactions. This network is enriched for Fanconi anemia genes (blue nodes). 5B) Model of FA complexes responding to a DNA lesion (vertical line) encountered by a replication fork (blue line=genomic DNA; green line=nascent DNA). Genes are color coded based on the performance of their knockouts in the screen. 5C) Western blot of FANCD2 response to 24-hour treatment with 1 μg/ml mitomycin C (MMC). Cells are treated with FA member sgRNAs or non-targeting-control (NTC). FANCD2 monoubiquitination is assessed as the ratio of FANCD2-L (long) to FANCD2-S (short) band intensities (relative L:S ratio). The ratio is graphed relative to NTC, MMC-treated cells. nd=not determined. Uncropped blot is shown in the Source Data. 5D) Clonogenic growth assay of LINE-1(+) RPE cells with sgRNAs targeting the same genes as in (C). n=3 independent experiments. P value calculated with a one-sided T test. 5E) Representative western blot of FANCD2 and FANCI in response to 72 hours expression of LINE-1 or luciferase in RPE. MMC treatment allows delineation of L (monoubiquitinated) and S (non-ubiquitinated) protein bands. Quantification to the right of n=2 independent experiments ±SEM. Uncropped blot is shown in the Source Data. 5F) Representative western blot of FANCD2 in response to 72 hours expression from plasmids with wildtype or mutant LINE-1 in HeLa cells. Quantification below of n=2 independent experiments ±SEM. Effect of wildtype LINE-1 is statistically significant as assessed by ANOVA (p=0.0143). Uncropped blot is shown in the Source Data. 5G) Left, representative images of FANCD2 foci (green) in EdU+ nuclei. Scale bar=6 μm. Right, quantification of FANCD2 foci in EdU+HeLa cells. Number of cells per group: untreated, n=134; HU, n=105; wildtype, n=109; RT (D702Y), n=101. Two-sided T tests were used for statistical comparisons. HU=hydroxyurea. RT=reverse transcriptase. ns=not significant. 5H) Left, γH2A.X and 53BP1 focus quantification in EdU+TP53^{KD} cells. Number of cells per group: Lucif, n=326; LINE-1, n=358; doxorubicin, n=431. Two-sided T tests were used for statistical comparisons. Right, representative images of γH2A.X (red), 53BP1 (green), EdU (cyan), and DAPI (blue). Scale bar=12 μm.

FIGS. 6A-6G. LINE-1 activity induces replication stress. 6A) Median count of sgRNAs targeting replication stress signaling genes ATRIP and the 9-1-1 complex (HUS1 and RAD1) during the screen. Error bars indicate 95% confidence intervals. 6B) Clonogenic assay of LINE-1(+) RPE cells (induced with 1 μg/ml doxycycline) with CRISPR-knockout of ATRIP compared to non-targeting-control (NTC). Error bars indicate SEM, n=3 independent experiments. P value is calculated with an unpaired two-sided T test. 6C) Clonogenic assay of LINE-1(+) RPE cells (induced with 1 μg/ml doxycycline) with drug inhibition of ATR kinase by 1 μM VE-821 compared to vehicle (DMSO). Error bars indicate SEM, n=3 independent experiments. P value is calculated with an unpaired two-sided T test. 6D) Western blot of RPA2 occupancy on chromatin induced by LINE-1 compared to luciferase control after 72 hours of expression in RPE. Chromatin-bound protein lysates were used. 1 μM MMC was used as a control to verify that these cells respond to replication stress. Uncropped blot is shown in the Source Data. 6E) Western blot of p-RPA S4/S8 after 72 hours of wildtype or mutant LINE-1 expression in HeLa cells. Relative signal intensity for n=2 independent experiments ±SEM is quantified. 1 μM MMC was used as a replication stress control and produces a gel shift in total RPA2 that is more subtly produced by WT LINE-1, which is the the hyperphosphorylated protein. Statistical significance is assessed by ANOVA (p=0.0007). Uncropped blot is shown in the Source Data. 6F) MMC dose-response clonogenic assay of LINE-1(+) cells or control. Molar concentration indicated on x-axis. Data are plotted as the mean viability relative to 100 pM ±SD, n=3 independent experiments. Two-sided T tests were used to compare relative viability at each dose. 6G) Median count of sgRNAs targeting fork protection (RADX) and fork restart (BLM, WRN, WRNIP1) genes. Median values are depicted with 95% Confidence Intervals.

FIG. 7. Model of LINE-1-induced replication stress. Collision of a replication fork, comprised of genomic DNA (dark blue) and newly synthesized DNA (green), with a LINE-1 insertion intermediate—an RNA:DNA hybrid made of LINE-1 mRNA (red) and LINE-1 cDNA (green). The LINE-1 insertion intermediate is recognized by the Fanconi Anemia pathway core complex and recruits and activates FANCD2 and FANCI, which are then monoubiquitinated. The stalled fork leads to an accumulation of RPA, which recruits ATR-ATRIP and the 9-1-1 (RAD9-HUS1-RAD1) complex, key replication stress signaling proteins. These coordinate the cell response to the replication stress, including phosphorylation of RPA. Failure to resolve this collision reduces cell fitness. A similar conflict could occur upstream of the lagging strand as well.

FIGS. 8A-8D. LINE-1 Peptide Detection in Tumor Mass Spectrometry Data. (8A) ORF1p peptides observed in CPTAC breast and ovarian tumors. Each column represents a tumor-derived MS dataset (102 fuchsia-colored columns for breast tumors and 176 sea foam-colored columns for ovarian tumors) analyzed for the presence of L1 ORF peptides. ORF1 peptides, displayed at the right, mark rows. A red tick indicates that the given peptide was detected as present in the according tumor sample (white space: peptide not detected). Highest quality PSMs that were observed for (8B, 8C) ORF1p and (8D) ORF2p are displayed. Precursor ion related peaks are shown in yellow, y-ions in red, b-ions in blue, and unassigned ions in black.

FIGS. 9A-9H: Production of monoclonal ORF2p antibodies. FIG. 9A: Expression constructs used to generate antigens for ORF2p antibody production. FIG. 9B: Coomassie-stained protein electrophoresis gels illustrating purity of ORF2p antigens used in antibody generation. FIG. 9C: Immunization strategy to produce rabbit monoclonal antibodies. FIG. 9D: Western blot detection of overexpressed ORF2p-3× Flag obtained from HEK-293TLD cells transfected with pLD561 (shown in panel a) using 5 different monoclonal antibodies (Ab) compared to anti-Flag. FIG. 9E: Immunoprecipitation of ORF2p-3× Flag using 3 antibodies. FIG. 9F: Immunofluorescence imaging of HEK-293TLD cells expressing ORF2p-3× Flag showing co-localization with anti-Flag antibody. FIG. 9G: Immunohistochemistry of HEK-293TLD cells expressing ORF2p-3× Flag with 4 monoclonal antibodies compared to anti-Flag. FIG. 9H: Above, overview of PhIP-Seq. A phage library expresses protein epitopes from the protein-coding genome, which are affinity purified with ORF2p antibodies. DNA sequences are then isolated and sequenced to identify the genes encoding the peptides. Below, results from five monoclonal antibodies targeting ORF2p. In each instance, the greatest affinity of the ORF2p monoclonal antibodies is for peptides encoded by L1Hs ORF2p peptides. EN=endonuclease, RT=reverse transcriptase, MBP=mannose binding protein, SUMO=small ubiquitin-like modification.

FIGS. 10A-10F. ORF2 mAbs detect endogenous L1Hs. (10A) Epitopes identified by five ORF2 mAbs are indicated along the linear sequences of the ORF2 protein. (10B) Immunoprecipitation (IP) blockade of ORF2p pulldown can be achieved by pre-incubating ORF2 mAbs with blocking peptides identified in (10A). (10C) ORF2 mAb epitopes are highly conserved among both full-length and ORF2-intact L1Hs sequences in the human genome. (10D) Western blot measuring the ability of the MT5 antibody to detect an L1Hs polymorphism at amino acid position 990 reveals that the antibody can detect both alleles. (10E) Epitope % identities among 31 ‘hot’ or highly active L1Hs sequences as reported by Brouha et al. (10F) Western blot of whole cell lysates (WCL) from several ORF1p negative and ORF1p positive cancer cell lines fails to detect ORF2 protein with two different ORF2 mAbs. HEK-293T_{LD} cells expressing ORF2-3× Flag are included as a positive control.

FIGS. 11A-11F. Protein Staining and Western Blotting of anti-ORF1p IPs and extracts. (11A) Three tumors (labeled TOP, LEFT) were used as starting material for ORF1p affinity isolations (α-ORF1p T), including mock-capture controls using mouse IgG affinity medium with tumor extracts (mIgG), and matched normal tissue with anti-ORF1p affinity medium (α-ORF1p N). The eluted material was electrophoresed (4-12% Bis-Tris NuPAGE) and Coomassie G-250 stained and a 200 ng BSA standard is displayed as a staining intensity gauge. Each lane contains a 200 mg-scale isolation using 10 μl of affinity medium. Several bands were cut and analyzed by LC-MS/MS—the highest-ranking proteins are listed (see Methods) (11B) Tumor A anti-ORF1p affinity capture was repeated using a slightly modified procedure (see Methods). 30% of 100 mg-scale affinity isolations using 15 μl of affinity medium have been electrophoresed and Sypro Ruby stained. (11C) Comparison of ORF1p yield from anti-ORF1p affinity isolations. pLD401 is a codon-optimized L1 sequence (OrfeusHs), ectopically expressed in HEK-293T_{LD}. Here, 80% of 100 mg-scale affinity isolations using 10 μl of affinity medium have been electrophoresed and Coomassie G-250 stained. (11D) Western blotting of the same materials used in (11C), including Tumor C and matched normal tissues. Here, 25 μg of the whole cell extract have been probed for ORF2p, ORF1p, and GAPDH as a control. 10% of α-ORF1p affinity isolates have also been probed for ORF2p and ORF1p. (11E) A collection of cell lines were assessed by anti-ORF1p affinity capture. pMT302 is derived from a naturally occurring L1 sequence (L1RP), ectopically expressed in HEK-293T_{LD}. pLD222 is a plasmid harboring a doxycycline-inducible GFP construct ectopically expressed in HEK-293TLD; here included as a control for pMT302. (11F) IHC using α-ORF1p on (LEFT) Tumor B and (RIGHT) Tumor C. α-ORF2p clones MT5 (panel D) and MT9 (panel E) are described in this study (see FIGS. 9 and 10).

FIGS. 12A-12C. Label-free Quantitative IP-MS analysis. A legend appears at the bottom: attention is drawn to hits observed previously by I-DIRT, matches to L1RE1 (Uniprot: ORF1p consensus), and candidate non-consensus ORF1p sequences. Gene symbols corresponding to tumor-specific, quantified proteins are displayed on each plot with the following criteria: 1. the protein exhibited statistical significance in the IP (see Methods) with a loge fold change ≥2 and also exhibited statistical significance in another IP from this study with loge fold change ≥1; or 2. the protein was previously determined specific by I-DIRT or was highlighted in other literature (discussed in the main text) and exhibited statistical significance. (12A) Tumor A, these IPs (set 1 and 2) differ in several experimental parameters (see Methods); both sets use a mock IP control (mouse IgG). (12B) Tumor B, two distinct controls were used: (LEFT) mIgG IP, (RIGHT) matched normal liver, α-ORF1p IP. (12C) Tumor C, controls as for Tumor B with matched normal colon.

FIGS. 13A and 13B. Treatment with a PKR inhibitor (PKRi, CAS 608512-97-6-Sigma cat#527450) causes a dose-dependent increases the accumulation of ORF2p (relative to ORF1p and tubulin) by day 5 of treatment. (Day 1 and 3 show little effect. The effect is sustained at day 10. Higher doses of the drug are toxic). The cells used are RPE cells that have a doxycycline (Dox) controlled LINE-1 expression construct. (13A.) Western blot showing ORF2p, ORF1p, and tubulin levels on day 5. Addition of Dox induces LINE-1 expression. Addition of PKRi enhances relative ORF2p expression levels. (13B.) Quantification of ORF2p expression (relative to tubulin) by Western blot as a function PKRi inhibitor dose on different days of the experiment.

### DETAILED DESCRIPTION

The present inventors now show that ORF2p protein is suppressed in the cytosol of many different types of cancer. The inventors have created novel antibodies to several ORF2p antigenic epitopes and have mapped them to specific sequences of the protein. The novel antibodies can be used to detect ORF2p expression in a variety of cell and cell-free media. In addition, the inventors now show that ORF2p is inhibitory to cell growth and that upregulation of ORF2p protein is possible using a small molecule targeting PKR. Such an approach therefore can be useful in identifying new drugs and therapies which can increase the expression of ORF2p in neoplasias and have chemotherapeutic effect.

In accordance with an embodiment, the present invention provides a method for treatment of a neoplasia in a cell or population of cells comprising increasing expression of ORF2p in the cell or population of cells.

As discussed, in preferred aspects, a neoplasia or patient suffering from a neoplasia will be identified and selected for treatment based on ORF2p levels, particularly reduced ORF2p expression relative to ORF1p expression. For instance, a tissue sample may be assessed for ORF2p expression by use of mass spectrometry or an antibody disclosed herein. See, for instance, the exemplary procedures at Examples 9 and 10 which follow.

As discussed above, ORF1p expression may be assessed in cells or a tissue sample including relative to levels of ORF2p expression. A variety of approaches may be utilized for assessing ORF1p expression including use of ORF1p antibodies. Suitable ORF1p antibodies for use in the present methods and systems have been disclosed in Rodić et al., Am J Pathol. 2014 May;184(5):1280-6. doi: 10.1016/j.ajpath.2014.01.007. Epub 2014 Mar. 6 and are commercially available from EMD Millipore (catalog #MABC1152). ORF1p antibodies also are commercially available from vendors such as Abcam (see Abcam ORF1p monoclonal antibodies ab216324, ab230966, ab2455249, ab246317 and ab246320) Antibody-based assays including a Western blot for example of a biopsy sample can be suitably employ for assessing ORF1p and ORF2p expression levels in a sample.

In accordance with an embodiment, the present invention provides a method for treatment of a neoplasia in a cell or population of cells comprising increasing expression of ORF2p in the cell or population of cells by targeting (e.g., inhibition of) protein kinase RNA activated (PKR) in the cell or population of cells.

In accordance with an embodiment, the present invention provides a method for treatment of a neoplasia in a cell or population of cells comprising increasing expression of ORF2p in the cell or population of cells by targeting (e.g. inhibition of) protein kinase RNA activated (PKR) in the cell or population of cells in combination with one or more additional chemotherapeutic agents.

In accordance with an embodiment, the present invention provides a method for treatment of a neoplasia in a subject in need thereof, comprising administering to the subject a biologically active agent which increases expression of ORF2p in the neoplasia of the subject.

In accordance with an embodiment, the present invention provides a method for treatment of a neoplasia in a subject in need thereof, comprising administering to the subject a biologically active agent which increases expression of ORF2p in the neoplasia of the subject in combination with one or more additional chemotherapeutic agents.

In accordance with an embodiment, the present invention provides a method for treatment of a neoplasia in a subject in need thereof, comprising increasing expression of ORF2p in the neoplasia of the subject by targeting (e.g. inhibition of) protein kinase RNA activated (PKR) in the neoplasia of the subject.

In accordance with an embodiment, the present invention provides a method for treatment of a neoplasia in a subject in need thereof, comprising increasing expression of ORF2p in the neoplasia of the subject by targeting (e.g. inhibition of) protein kinase RNA activated (PKR) in the neoplasia of the subject in combination with one or more additional chemotherapeutic agents.

Protein kinase R (PKR) is a serine-threonine kinase (551 amino acid long) encoded in humans by the EIF2AK2 gene located on chromosome 2, which plays a major role in central cellular processes such as mRNA translation, transcriptional control, regulation of apoptosis, and proliferation (García et al., 2007). In accordance with such preponderant role, PKR dysregulation has been implicated in cancer, neurodegeneration (Segev et al., 2013, 2015; Stern et al., 2013), inflammation, and metabolic disorders (Segev et al., 2016; Garcia-Ortega et al., 2017). This kinase, which is constitutively and ubiquitously expressed in vertebrate cells, is not found in plants, fungi, protists, or invertebrates (Taniuchi et al., 2016). PKR was first cloned in 1990 at the Pasteur Institute (Meurs et al., 1990; Watanabe et al., 2018), and is also known as Protein kinase RNA-activated; and interferon-induced, double-stranded RNA-domain kinase (Hugon et al., 2009).

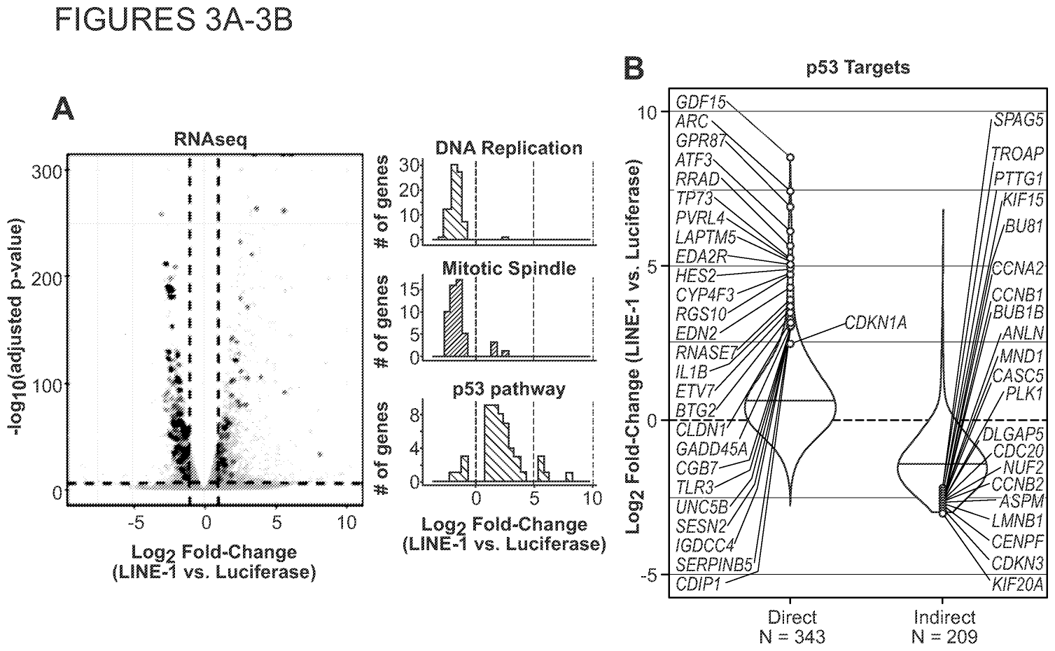

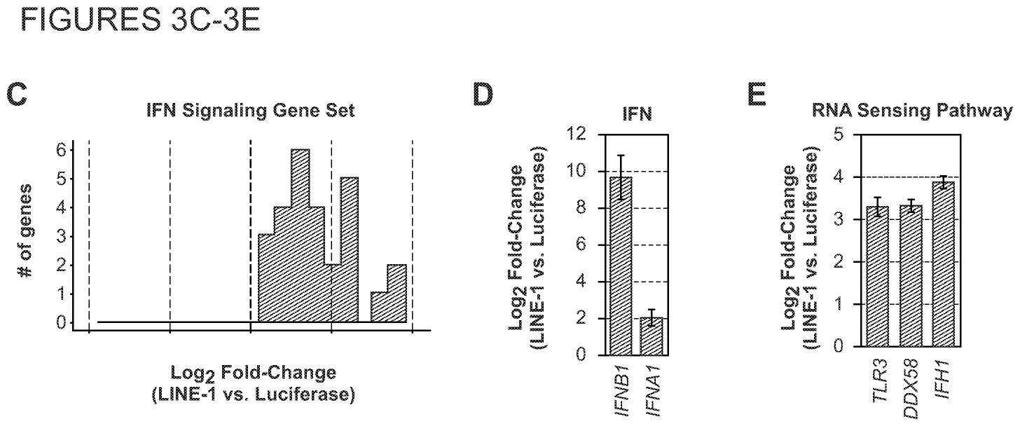

In the present inventive system, PKR (EIF 2AK2) mRNA expression is being induced in cells by our expression of LINE-1. We have determined that LINE-1 can activate interferon responses (FIG. 3) and PKR mRNA increases with that program. Without being held to any particular theory, over time, ORF2p expression levels decrease, potentially through PKR dependent mechanisms.

Protein kinase R serves as a central hub for the detection of cellular stress signals and response to them, and is thus expected to be regulated by different stress-response pathways. In accord with this notion, the canonical activator of PKR is double-stranded RNA (an obligatory feature of the replication process of RNA viruses), rendering PKR as a pattern recognition receptor endowed with cell function modulatory abilities. The central role of PKR in mediating anti-viral responses is also evidenced by the high degree of positive selection exhibited by coding sequence, indicative of the arms race against the pathogens it encounters and combats (Elde et al., 2009; Rothenburg et al., 2009; Carpentier et al., 2016). However, PKR can also be activated by other factors, for example, heat shock proteins, growth factors (e.g., PDGF), and heparin (Li et al., 2006). PKR is also activated in response to numerous insults, including non-viral pathogens (bacterial lipopolysaccharide, which activates the toll-like receptor 4 pathway), nutrition or energy excess, cytokines (e.g., TNF-α, IL-1, IFN-γ), calcium, reactive oxygen species, irradiation (presumably by inducing DNA damage), mechanical stress, and endoplasmic reticulum stress resulting from the presence of a large quantity of unfolded proteins [caused, e.g., by tunicamycin, arsenite, thapsigargin, or H2O2, which in turn activate the PKR activator protein.

The most widely used pharmacological PKR inhibitor is the highly potent small molecule imidazolo-oxindole C16, also known as PKRi, which targets the ATP binding site of PKR. C16 has an IC50 of about 200 nM in vitro, and is typically used at doses of 200-500 nM in vitro for 1 h. Another less specific pharmacological inhibitor of PKR is the 2-aminopurine (2-AP) compound, which competes for ATP at the ATP binding site of PKR, and thereby inhibits its phosphorylation. This compound is less potent than C16, and is used in vitro at doses of 4-10 mM for 4 h. Other inhibitors include 6-amino-3-methyl-2-oxo-N-phenyl-2,3-dihydro-1H-benzo[d]imidazole-1-carboxamide, and 3-methyl-6-(methylsulphonamido)-2-oxo-N-phenyl-2,3-dihydro-1H-benzo[d]imidazole-1-carboxamide (Mol. Diversity, 20 805-819 (2016).

In some embodiments, the PKR inhibitor is selected from the group consisting of 2-aminopurine, 6,8-dihydro-8-(1H-imidazol-5-ylmethylene)-7H-pyrrolo[2,3-g]benzothiazol-7-one, 6-amino-3-methyl-2-oxo-N-phenyl-2,3-dihydro-1H-benzo[d]imidazole-1-carboxamide, and 3-methyl-6-(methylsulphonamido)-2-oxo-N-phenyl-2,3-dihydro-1H-benzo[d]imidazole-1-carboxamide.

In some embodiments the biologically active agent which increases expression of ORF2p is a small nucleic acid sequence such as an anti-mIR or SINEUP sequence. In particular, a SINEUP nucleic acid molecule is specific to the inter-ORF region of LINE-1 around the start codon of the mRNA encoding ORF2p and increases translation of the ORF2p protein and its expression.

In some embodiments the biologically active agent which prevents clearance of ORF2p protein in a cell or population of cells by inhibition of proteasomes or ubiquinylation of ORF2p in the cell.

In accordance with one or more embodiments, the present invention provides a method for increasing ORF2p expression in a cell by contacting the cell with a proteasomal inhibitor. In some embodiments, the proteasomal inhibitor can include compounds that inhibit one or more activities of a proteasome such as, but not limited to, peptide aldehydes, peptide boronates, and nonpeptide inhibitors. In certain embodiments, the proteasomal inhibitor can include Epoxomicin, Lactacystin, Bortezomib, MG-132, Carfilzomib, MLN9708, Ixazomib, PI-1840, ONX-0914, Oprozomib, CEP-18770, and Gabexate Mesylate, for example.

Additional suitable proteasomal inhibitors for use in the present methods and compositions may include epigallocatechin-3-gallate, salinosporamide A, carfilzomib, MLN9708, epoxomicin, MG132, Ixazomib. Additional non-limiting example of proteasomal inhibitors can be found, e.g., in U.S. Pat. No. 8,809,283; US Patent Publication 2011/0009332; International Patent Publications WO 2014/182744; WO1999/037666; and European Patent 1895971, all of which are incorporated by reference herein in its entirety.

In accordance with one or more embodiments of the present invention, it will be understood that the types of neoplasia diagnosis which may be made, using the methods provided herein, is not necessarily limited.

In some embodiments, the neoplasia to be treated is positive for LINE-1 expression. In other embodiments, the neoplasia to be treated is positive for LINE-1-encoded ORF1p protein.

Therefore, in accordance with another embodiment, the present invention provides a method for diagnosis of a neoplasia as being susceptible to treatment with increasing expression of ORF2p with detection of ORF1p expression in the neoplasia.

As used herein, the term “treat,” as well as words stemming therefrom, includes diagnostic and preventive as well as disorder remitative treatment.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of neoplasia in a mammal. Furthermore, the treatment or prevention provided by the inventive method can include treatment or prevention of one or more conditions or symptoms of the disease, e.g., neoplasia, being treated or prevented. Also, for purposes herein, “prevention” can encompass delaying the onset of the disease, or a symptom or condition thereof.

As used herein, the term “biologically active agent” any compound, biologic, e.g. drugs, inhibitors, proteins, cytokines, or stem cells. An active agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed. In some embodiments, the biologically active agent increases expression of ORF2p in a cell or population of cells, or suppresses the inhibition of expression of ORF2p in a cell or population of cells.

Non-limiting examples of biologically active agents include following: ADAR inhibitors, adrenergic blocking agents, anabolic agents, androgenic steroids, antacids, anti-asthmatic agents, anti-allergenic materials, anti-cholesterolemic and anti-lipid agents, anti-cholinergics and sympathomimetics, anti-coagulants, anti-convulsants, anti-diarrheal, anti-emetics, anti-hypertensive agents, anti-infective agents, anti-inflammatory agents such as steroids, non-steroidal anti-inflammatory agents, anti-malarials, anti-manic agents, anti-nauseants, anti-neoplastic agents, anti-obesity agents, anti-parkinsonian agents, anti-pyretic and analgesic agents, anti-spasmodic agents, anti-thrombotic agents, anti-uricemic agents, anti-anginal agents, antihistamines, anti-tussives, appetite suppressants, ATR inhibitors, benzophenanthridine alkaloids, biologicals, cardioactive agents, cerebral dilators, CHK1 inhibitors, coronary dilators, decongestants, diuretics, diagnostic agents, DNA damaging agents, DNA repair inhibitors, epigenetic modulators, erythropoietic agents, estrogens, expectorants, gastrointestinal sedatives, agents, hyperglycemic agents, hypnotics, hypoglycemic agents, integrated stress response inhibitor (ISRIB), ion exchange resins, laxatives, metformin, mineral supplements, mitotics, mucolytic agents, growth factors, neuromuscular drugs, nutritional substances, peripheral vasodilators, progestational agents, prostaglandins, proteasomal inhibitors, Protein kinase R activators and inhibitors, psychic energizers, psychotropics, sedatives, stimulants, thyroid and anti-thyroid agents, tranquilizers, ubiquinylation inhibitors, uterine relaxants, vitamins, WRN helicase inhibitors, antigenic materials, and prodrugs.

Specific examples of useful biologically active agents the above categories include: anti-neoplastics such as androgen inhibitors, antimetabolites, cytotoxic agents, and immunomodulators. More specifically, non-limiting examples of useful biologically active agents include the following therapeutic categories antineoplastic agents, such as alkylating agents, nitrogen mustard alkylating agents, nitrosourea alkylating agents, antimetabolites, purine analog antimetabolites, pyrimidine analog antimetabolites, hormonal antineoplastics, natural antineoplastics, antibiotic natural antineoplastics, and vinca alkaloid natural antineoplastics, such as carboplatin and cisplatin; carmustine (BCNU); methotrexate; fluorouracil (5-FU) and gemcitabine; goserelin, leuprolide, and tamoxifen, aldesleukin, interleukin-2, docetaxel, etoposide, interferon; paclitaxel, other taxane derivatives, tretinoin (ATRA); bleomycin, dactinomycin, daunorubicin, doxorubicin, and mitomycin; vinblastine and vincristine.

The dose of the compositions of the present invention also will be determined by the existence, nature and extent of any adverse side effects that might accompany the administration of a particular composition. Typically, an attending physician will decide the dosage of the pharmaceutical composition with which to treat each individual subject, taking into consideration a variety of factors, such as age, body weight, general health, diet, sex, compound to be administered, route of administration, and the severity of the condition being treated. By way of example, and not intending to limit the invention, the dose of the pharmaceutical compositions of the present invention can be about 0.001 to about 1000 mg/kg body weight of the subject being treated, from about 0.01 to about 100 mg/kg body weight, from about 0.1 mg/kg to about 10 mg/kg, and from about 0.5 mg to about 5 mg/kg body weight. In another embodiment, the dose of the pharmaceutical compositions of the present invention can be at a concentration from about 1 nM to about 10,000 nM, preferably from about 10 nM to about 5,000 nM, more preferably from about 100 nM to about 500 nM.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

In accordance with an embodiment of the present invention, the medicament for treating a disease in a subject can encompass many different formulations known in the pharmaceutical arts, including, for example, intravenous and sustained release formulations. With respect to the inventive methods, the disease can include cancer. Cancer can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer.

In another embodiment, the term “administering” means that at least one or more pharmaceutical compositions of the present invention are introduced into a subject, preferably a subject receiving treatment for a disease, and the at least one or more compositions are allowed to come in contact with the one or more disease related cells or population of cells.

As used herein, the term “treat,” as well as words stemming therefrom, includes diagnostic and preventative as well as disorder remitative treatment.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

Further examples of biologically active agents include, without limitation, enzymes, receptor antagonists or agonists, hormones, growth factors, autogenous bone marrow, antibiotics, antimicrobial agents, and antibodies. The term “biologically active agent” is also intended to encompass various cell types and genes that can be incorporated into the compositions of the invention.

In certain embodiments, the subject compositions comprise about 1% to about 75% or more by weight of the total composition, alternatively about 2.5%, 5%, 10%, 20%, 30%, 40%, 50%, 60% or 70%, of a biologically active agent.

The “therapeutically effective amount” of the pharmaceutical compositions to be administered will be governed by such considerations, and can be the minimum amount necessary to prevent, ameliorate or treat a disorder of interest. As used herein, the term “effective amount” is an equivalent phrase refers to the amount of a therapy (e.g., a prophylactic or therapeutic agent), which is sufficient to reduce the severity and/or duration of a disease, ameliorate one or more symptoms thereof, prevent the advancement of a disease or cause regression of a disease, or which is sufficient to result in the prevention of the development, recurrence, onset, or progression of a disease or one or more symptoms thereof, or enhance or improve the prophylactic and/or therapeutic effect(s) of another therapy (e.g., another therapeutic agent) useful for treating a disease, such as cancer.

In accordance with another embodiment, the present invention provides methods of treating cancer in a subject comprising administering to the mammal a therapeutically effective amount of the composition of the present invention sufficient to slow, stop or reverse the cancer in the subject.

In an embodiment, the methods of the present invention can include the biologically active agent and/or chemotherapeutic agent in conjunction with a carrier. The carrier is preferably a pharmaceutically acceptable carrier. With respect to pharmaceutical compositions, the carrier can be any of those conventionally used and is limited only by chemico-physical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

In accordance with an embodiment, the present invention provides a monoclonal antibody that specifically binds ORF2p antigenic epitopes.

In some embodiments, the ORF2p antigenic epitope is selected from the group consisting of DRSTRQ (SEQ ID NO: 1), LHQADLID (SEQ ID NO: 2), KASRRQEITKIRAE (SEQ ID NO: 3), KELEKQEQT (SEQ ID NO: 4), QDIGVGKD (SEQ ID NO: 5).

In accordance with an embodiment, the present invention provides the use of a monoclonal antibody that specifically binds ORF2p antigenic epitopes to detect expression of ORF2p in a cell or population of cells comprising administration of one or more of said monoclonal antibodies to the cell or population of cells.

In some embodiments, the ORF2p antigenic epitope is selected from the group consisting of DRSTRQ (SEQ ID NO: 1), LHQADLID (SEQ ID NO: 2), KASRRQEITKIRAE (SEQ ID NO: 3), KELEKQEQT (SEQ ID NO: 4), QDIGVGKD (SEQ ID NO: 5).

The term “antigen” or “antigenic epitope” as used herein refers to any molecule (e.g., protein, peptide, lipid, carbohydrate, etc.) solely or predominantly expressed or over-expressed by a target cell of interest, such that the antigen is associated with the target cell.

The term “polypeptide” as used herein includes oligopeptides and refers to a single chain of amino acids connected by one or more peptide bonds.

The term “a peptide or polypeptide fragment thereof, capable of being cleaved by a specific protease” as used herein, means an amino acid sequence which is specifically recognized by a protease enzyme and specifically binds and hydrolytically cleaves that amino acid sequence. The peptide sequence can be any sequence of between about 3 to about 20 amino acids in length, which is known to be cleaved by a known protease.

The term “functional portion” when used in reference to a monoclonal antibody or antigenic epitope refers to any part or fragment, which part or fragment retains the biological activity of which it is a part (the parent molecule, antibody, or antigen). Functional portions encompass, for example, those parts that retain the ability to specifically bind to the antigen (e.g., in an MHC-independent manner), or detect, treat, or prevent the disease, to a similar extent, the same extent, or to a higher extent, as the parent molecule. In reference to the parent molecule, the functional portion can comprise, for instance, about 10%, 25%, 30%, 50%, 68%, 80%, 90%, 95%, or more, of the parent molecule.

The functional portion can comprise additional amino acids at the amino or carboxy terminus of the portion, or at both termini, which additional amino acids are not found in the amino acid sequence of the parent molecule. Desirably, the additional amino acids do not interfere with the biological function of the functional portion, e.g., specifically binding to a cancer antigen, having the ability to detect cancer, treat or prevent cancer, etc. More desirably, the additional amino acids enhance the biological activity, as compared to the biological activity of the parent molecule.

By “protein” is meant a molecule comprising one or more polypeptide chains.

In this regard, the invention also provides an immunoconjugate molecule comprising at least one of the polypeptides described herein along with at least one other polypeptide. The other polypeptide can exist as a separate polypeptide of the fusion protein, or can exist as a polypeptide, which is expressed in frame (in tandem) with one of the inventive polypeptides described herein. The other polypeptide can encode any peptidic or proteinaceous molecule, or a portion thereof. Suitable methods of making fusion proteins are known in the art, and include, for example, recombinant methods. See, for instance, Choi et al., Mol. Biotechnol. 31: 193-202 (2005).

As used herein, “recombinant antibody” refers to a recombinant (e.g., genetically engineered) protein comprising at least one of the polypeptides of the invention and a polypeptide chain of an antibody, or a portion thereof. The polypeptide chain of an antibody, or portion thereof, can exist as a separate polypeptide of the recombinant antibody. Alternatively, the polypeptide chain of an antibody, or portion thereof, can exist as a polypeptide, which is expressed in frame (in tandem) with the polypeptide of the invention. The polypeptide of an antibody, or portion thereof, can be a polypeptide of any antibody or any antibody fragment, including any of the antibodies and antibody fragments described herein.

Included in the scope of the invention are functional variants of the inventive monoclonal antibodies, polypeptides, and proteins described herein. The term “functional variant” as used herein refers to an antibodies, polypeptides, or proteins having substantial or significant sequence identity or similarity to a parent antibodies, polypeptides, or proteins, which functional variant retains the biological activity of the antibodies, polypeptides, or proteins of which it is a variant. In reference to the parent antibodies, polypeptides, or proteins, the functional variant can, for instance, be at least about 30%, 50%, 75%, 80%, 90%, 98% or more identical in amino acid sequence to the parent antibodies, polypeptides, or proteins.

The functional variant can, for example, comprise the amino acid sequence of the parent antibodies, polypeptides, and proteins with at least one conservative amino acid substitution. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc

Alternatively or additionally, the functional variants can comprise the amino acid sequence of the parent antibodies, polypeptides, and proteins with at least one non-conservative amino acid substitution. In this case, it is preferable for the non-conservative amino acid substitution to not interfere with or inhibit the biological activity of the functional variant. Preferably, the non-conservative amino acid substitution enhances the biological activity of the functional variant, such that the biological activity of the functional variant is increased as compared to the parent antibodies, polypeptides, and proteins.

The antibodies, polypeptides, and proteins of the invention (including functional portions and functional variants thereof) can be obtained by methods known in the art. Suitable methods of de novo synthesizing polypeptides and proteins are described in references, such as Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westwoood et al., Oxford University Press, Oxford, United Kingdom, 2000; and U.S. Pat. No. 5,449,752. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, NY 2001; and Ausubel et al., Current Protocols in Molecular Biology, Greene Publishing Associates and John Wiley & Sons, NY, 1994. Further, some of the antibodies, polypeptides, and proteins of the invention (including functional portions and functional variants thereof) can be isolated and/or purified from a source, such as a plant, a bacterium, an insect, a mammal, e.g., a rat, a human, etc. Methods of isolation and purification are well-known in the art. Alternatively, the antibodies, polypeptides, and proteins described herein (including functional portions and functional variants thereof) can be commercially synthesized by companies, such as Synpep (Dublin, Calif.), Peptide Technologies Corp. (Gaithersburg, Md.), and Multiple Peptide Systems (San Diego, Calif.). In this respect, the inventive antibodies, polypeptides, and proteins can be synthetic, recombinant, isolated, and/or purified.

The antibody can be in monomeric or polymeric form. Also, the antibody or fragments thereof, can have any level of affinity or avidity for the target cell or population of cell antigen(s). Desirably, the antibody is specific for the functional portion of the target cell or population of cells, such that there is minimal cross-reaction with other cells or populations of cells.

Methods for generating humanized antibodies are well known in the art and are described in detail in, for example, Janeway et al., supra, U.S. Pat. Nos. 5,225,539, 5,585,089 and 5,693,761, European Patent No. 0239400 B1, and United Kingdom Patent No. 2188638. Humanized antibodies can also be generated using the antibody resurfacing technology described in U.S. Pat. No. 5,639,641 and Pedersen et al., J. Mol. Biol., 235, 959-973 (1994).

The invention also provides antigen binding portions of any of the antibodies described herein.

Also, the antibody, or antigen binding portion thereof, can be modified to comprise a detectable label, such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

The antibodies of the present invention can be formulated into a composition, such as a pharmaceutical composition. In this regard, the invention provides a pharmaceutical composition comprising any of the antibodies, polypeptides, proteins, functional portions, functional variants, nucleic acids, expression vectors, and a pharmaceutically acceptable carrier. The inventive pharmaceutical compositions containing any of the inventive antibodies can comprise more than one antibody.

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### EXAMPLES

Cell Lines

Tet-On 3G HEK293 cells (ClonTech) were used, Tet-On HEK293T (JD Boeke), Tet-On 3G Hela (ClonTech), HEK293FT (AJ Holland), hTERT-RPE1^{puroS} (AJ Holland), and hTERT-RPE1^{puroS}-Cₐₛ9 (AJ Holland). RPE cells have been authenticated by STR profiling. Cells were grown in DMEM (293, HeLa) or DMEM/F12 with 1.5% sodium bicarbonate (RPE) with 10% Tetracycline-free Fetal Bovine Serum (Takara Bio USA). Cells were cultured at 37C, 5% CO₂. Antibiotic selection was performed with puromycin (1 μg/ml), G418 (400 μg/ml), or blasticidin (10 μg/ml). Doxycycline was used at 1 μg/ml unless otherwise stated. Cells were tested and mycoplasma negative.

TP53^{KD} Generation

For shRNA growth experiments, TP53^{WT} RPE-Cas9 cells were transduced with pOT-p53-shRNA-TagRFP or pSicoR-mCh_empty, then transfected with LINE-1 or eGFP plasmids. To generate monoclonal knockout cells, RPE-Cas9 cells were transduced with pOT-p53-shRNA-TagRFP lentivirus and single RFP+ cells were sorted by a FACS Aria into 96-well plates. Monoclonal cell lines were screened for p53 knockdown by western blot in cells treated with 200 ng/ml doxorubicin.

TP53^{KD} Generation

For shRNA growth experiments, TP53^{WT} RPE-Cas9 cells were transduced with pOT-p53-shRNA-TagRFP or pSicoR-mCh_empty, then transfected with LINE-1 or eGFP plasmids. To generate monoclonal knockout cells, RPE-Cas9 cells were transduced with pOT-p53-shRNA-TagRFP lentivirus and single RFP+ cells were sorted by a FACS Aria into 96-well plates. Monoclonal cell lines were screened for p53 knockdown by western blot in cells treated with 200 ng/ml doxorubicin.

Viability Assessments

Viability was determined by clonogenic growth or CellTiter-Glo assay (Promega, Madison, Wis.). WT RPE were assessed by clonogenic growth by transfecting 1e5 cells with 2 μg eGFP (pDA083) or 3 μg LINE-1 (pDA077) plasmid to achieve equimolar ratios. Cells were split to 10 cm growth dishes and selected with G418 24 hours later. In Tet-On assays, 500 cells were plated and doxycycline was added to activate transgene expression. For MMC sensitivity experiments, cells were treated with 100 pM, 1 nM, 10 nM, and 100 nM for 24 hours on day 2 after plating. In VE-821 sensitivity, cells were treated with 1 μM drug or DMSO vehicle throughout the duration of the experiment. For assays in CRISPR knockout cells, knockout cell pools were generated by infecting TP53^{KD} Tet-On RPE cells with lentivirus encoding either non-targeting control or a gene targeting guide and selecting with puromycin for 1 week. For all assays, after 10-14 days of L1 or control expression, colonies were washed with PBS and fixed (6% gluteraldehyde, 0.5% crystal violet) for 10 minutes. Plates were rinsed in water and air dried, then imaged on a flatbed scanner. Colonies with >50 cells were counted.

A similar procedure was used for clonogenic assays in 293 cells, except that 1e5 cells were transfected with LINE-1 (pDA056) and blasticidin-selected, then 500 cells were plated on poly-D-lysine (Sigma) coated plates and LINE-1 was induced with 1 μg/ml doxycycline before colony fixation. Growth was quantified based on % plate confluence using ImageJ.

CellTiter-Glo assays were performed in 293T cells transfected with LINE-1 (pDA007), LINE-1 ORF2 H230A (pDA025), LINE-1 ORF2 D702Y (pDA034), LINE-1 ORF2 H230A/D702Y (pDA027), or empty vector (pDA019). 8,000 cells were plated per well and treated with doxycycline (0-1000 ng/ml) for 72 hours. CellTiter reagents were then added and luminescence was measured using a Glomax Multi+ Detection System (Promega, Madison, Wis.).

CRISPR Knockout Screening

We used the Brunello GPP pooled CRISPR knockout library packaged into lentivirus for screening. The library comprises 76,441 gRNAs targeting 19,114 genes, with 4 sgRNAs per gene. TP53^{WT}-Cas9 cells were transduced at 100-fold library representation at a multiplicity of infection (MOI) of 0.2, in duplicate. TP53^{KD}-Cas9 with LINE-1 or luciferase transgenes were transduced at 100-fold library representation at an MOI of 0.3, in triplicate. Knockout pools were puromycin-selected for 8 days. TP53^{WT}-Cas9 cells were transfected with LINE-1 (pDA077) or eGFP (pDA083) at 150-fold library representation and assayed for library representation at day 19. TP53^{KD}-Cas9 cells were started at 500-fold library representation and maintained at 200-fold representation during passages through day 27. For TP53^{KD}-Cas9 screens, cells were continuously doxycycline-treated and sampled every 4-5 days. Cells were lysed (50 mM Tris, 50 mM EDTA, 1% SDS, pH 8), incubated with RNase A and Proteinase K, and DNA was extracted by isopropanol precipitation. DNA concentrations were measured by Nanodrop. Library preparation was performed with a 1-step PCR by Q5 Hot-start polymerase master mix (cat#M0494, NEB, Ipswich, Mass. (98C for 30 seconds; 24 cycles: 98C for 5 seconds, 68C for 30 seconds, 72° C. for 30 seconds; 72° C. for 2 minutes; hold at 10° C.). See Table 1 for primer sequences. Barcoded libraries were quantified using the NEB Library Quant Kit and mixed to obtain equal coverage, then sequenced with single-end 75 base reads on an Illumina NextSeq 500.

TABLE 1

| | | Primer | |
|---|---|---|---|
| Method | Category | Name | Sequence (5′-3′) |
| PCR | Screen | GECKO_PCR2_F01 | AATGATACGGCGACCACCGAGATCTACACTCTTTCCCTACACG |
| | Library | | ACGCTCTTCCGATCTNNNNAAGTAGAGtcttgtggaaaggacgaaacaccg |
| | Preparation | | (SEQ ID NO: 6) |
| PCR | Screen | GECKO_PCR2_F02 | AATGATACGGCGACCACCGAGATCTACACTCTTTCCCTACACG |
| | Library | | ACGCTCTTCCGATCTNNNNNACACGATCtcttgtggaaaggacgaaacaccg |
| | Preparation | | (SEQ ID NO: 7) |
| PCR | Screen | GECKO_PCR2_F03 | AATGATACGGCGACCACCGAGATCTACACTCTTTCCCTACACG |
| | Library | | ACGCTCTTCCGATCTNNNNNNCGCGCGGTtcttgtggaaaggacgaaacacc |
| | Preparation | | g (SEQ ID NO: 8) |
| PCR | Screen | GECKO_PCR2_F04 | AATGATACGGCGACCACCGAGATCTACACTCTTTCCCTACACG |
| | Library | | ACGCTCTTCCGATCTNNNNNNNCATGATCGtcttgtggaaaggacgaaaca |
| | Preparation | | ccg (SEQ ID NO: 9) |
| PCR | Screen | GECKO_PCR2_F05 | AATGATACGGCGACCACCGAGATCTACACTCTTTCCCTACACG |
| | Library | | ACGCTCTTCCGATCTNNNNCGTTACCAtcttgtggaaaggacgaaacaccg |
| | Preparation | | (SEQ ID NO: 10) |
| PCR | Screen | GECKO_PCR2_F06 | AATGATACGGCGACCACCGAGATCTACACTCTTTCCCTACACG |
| | Library | | ACGCTCTTCCGATCTNNNNNTCCTTGGTtcttgtggaaaggacgaaacaccg |
| | Preparation | | (SEQ ID NO: 11) |
| PCR | Screen | GECKO_PCR2_F07 | AATGATACGGCGACCACCGAGATCTACACTCTTTCCCTACACG |
| | Library | | ACGCTCTTCCGATCTNNNNNNAACGCATTtcttgtggaaaggacgaaacacc |
| | Preparation | | g (SEQ ID NO: 12) |
| PCR | Screen | GECKO_PCR2_F08 | AATGATACGGCGACCACCGAGATCTACACTCTTTCCCTACACG |
| | Library | | ACGCTCTTCCGATCTNNNNNNNACAGGTATtcttgtggaaaggacgaaaca |
| | Preparation | | ccg (SEQ ID NO: 13) |
| PCR | Screen | GECKO_PCR2_F09 | AATGATACGGCGACCACCGAGATCTACACTCTTTCCCTACACG |
| | Library | | ACGCTCTTCCGATCTNNNNAGGTAAGGtcttgtggaaaggacgaaacaccg |
| | Preparation | | (SEQ ID NO: 14) |
| PCR | Screen | GECKO_PCR2_F10 | AATGATACGGCGACCACCGAGATCTACACTCTTTCCCTACACG |
| | Library | | ACGCTCTTCCGATCTNNNNNAACAATGGtcttgtggaaaggacgaaacaccg |
| | Preparation | | (SEQ ID NO: 15) |
| PCR | Screen | GECKO_PCR2_F11 | AATGATACGGCGACCACCGAGATCTACACTCTTTCCCTACACG |
| | Library | | ACGCTCTTCCGATCTNNNNNNACTGTATCtcttgtggaaaggacgaaacacc |
| | Preparation | | g (SEQ ID NO: 16) |
| PCR | Screen | GECKO_PCR2_F12 | AATGATACGGCGACCACCGAGATCTACACTCTTTCCCTACACG |
| | Library | | ACGCTCTTCCGATCTNNNNNNNAGGTCGCAtcttgtggaaaggacgaaaca |
| | Preparation | | ccg (SEQ ID NO: 17) |
| PCR | Screen | GECKO_PCR2_R02 | CAAGCAGAAGACGGCATACGAGATACACGATCGTGACTGGAG |
| | Library | | TTCAGACGTGTGCTCTTCCGATCTNNNNNTCTACTATTCTTTCC |
| | Preparation | | CCTGCACTGT (SEQ ID NO: 18) |
| PCR | Cloning | SB- | GTACCACTTCCTACCCTCGAAAGGCCTCTGATGGGCAAGAAGC |
| | | ORFeus-5 | AGAACCG (SEQ ID NO: 19) |
| PCR | Cloning | SB- | TCATGTCTATCGATGGAAGCTTGGCCTGACATCAGTTGCCGCC |
| | | ORFeus-3 | GATCAGGC (SEQ ID NO: 20) |
| PCR | qRT-PCR | DA.q13.CDKN1A.left | TGTCTTGTACCCTTGTGCCT (SEQ ID NO: 21) |
| PCR | qRT-PCR | DA.q14.CDKN1A.right | AAGATGTAGAGCGGGCCTTT (SEQ ID NO: 22) |
| PCR | qRT-PCR | DA.q45.GAPDH.forward | GGATTTGGTCGTATTGGG (SEQ ID NO: 23) |
| PCR | qRT-PCR | DA.q46.GAPDH.reverse | GGAAGATGGTGATGGGATT (SEQ ID NO: 24) |
| PCR | qRT-PCR | DA.q.IFNB1.forward | AGCTGCTTAATCTCCTCAGGG (SEQ ID NO: 25) |
| PCR | qRT-PCR | DA.q.IFNB1.reverse | TCTCCTGTTGTGCTTCTCCA (SEQ ID NO: 26) |
| PCR | qRT-PCR | DA.q.TLR3.forward | TGTCTCATAATGGCTTGTCATCT (SEQ ID NO: 27) |
| PCR | qRT-PCR | DA.q.TLR3.reverse | GGCCAAATAATCTTCCAATTGCG (SEQ ID NO: 28) |
| PCR | qRT-PCR | DA.q.IFIT2.forward | AAACAACTGCTCCATCTGCG (SEQ ID NO: 29) |
| PCR | qRT-PCR | DA.q.IFIT2.reverse | TGCAAAGCCTCAGAATCTGC (SEQ ID NO: 30) |
| PCR | qRT-PCR | DA.q.IFIT1.forward | GCTTTCAAATCCCTTCCGCT (SEQ ID NO: 31) |
| PCR | qRT-PCR | DA.q.IFIT1.reverse | TAGGCAGAGATCGCATACCC (SEQ ID NO: 32) |
| PCR | qRT-PCR | DA.q15.ORFeus.left | AAGATCATCCGCGCCATCTA (SEQ ID NO: 33) |
| PCR | qRT-PCR | DA.q16.ORFeus.right | TCAGCTTCACCTCCTCCTTG (SEQ ID NO: 34) |
| CRISPR | Non- | GPP_76767 | CCATTCACAATCCCACTACA (SEQ ID NO: 35) |
| oligo | target- | | |
| | control | | |
| CRISPR | Non- | GPP_77342 | TGAGCATTCGTAGCCCAGCA (SEQ ID NO: 36) |
| oligo | target- | | |
| | control | | |
| CRISPR | TP53 | GPP_19455 | GATCCACTCACAGTTTCCAT (SEQ ID NO: 37) |
| oligo | Knockout | | |
| CRISPR | TP53 | GPP_19456 | GGTGCCCTATGAGCCGCCTG (SEQ ID NO: 38) |
| oligo | Knockout | | |
| CRISPR | CDKN1A | GPP_02927 | GTCACCGAGACACCACTGGA (SEQ ID NO: 39) |
| oligo | Knockout | | |
| CRISPR | CDKN1A | GPP_02925 | AGTCGAAGTTCCATCGCTCA (SEQ ID NO: 40) |
| oligo | Knockout | | |
| CRISPR | FANCM | GPP_46254 | TGACGGTGGTTACAACACGC (SEQ ID NO: 41) |
| oligo | Knockout | | |
| CRISPR | FANCA | GPP_06027 | GACACACAGAACCTTCCGAG (SEQ ID NO: 42) |
| oligo | Knockout | | |
| CRISPR | FANCL | GPP_41632 | CGAGATGAATCCCTCATACA (SEQ ID NO: 43) |
| oligo | Knockout | | |
| CRISPR | FANCI | GPP_41960 | TATGACTGTATTCTTATACC (SEQ ID NO: 44) |
| oligo | Knockout | | |
| CRISPR | FANCD2 | GPP_06034 | AGTTGACTGACAATGAGTCG (SEQ ID NO: 45) |
| oligo | Knockout | | |
| CRISPR | ATRIP | GPP_52946 | TCCTAGGAAAAACCCTTCTG (SEQ ID NO: 46) |
| oligo | Knockout | | |
| CRISPR | PPHLN1 | GPP_38975 | AGGTGTTAGACAAACCCAGT (SEQ ID NO: 47) |
| oligo | Knockout | | |
| CRISPR | TASOR | GPP_32305 | GGAAAACGAAATAACTCAAG (SEQ ID NO: 48) |
| oligo | Knockout | | |
| CRISPR | MPP8 | GPP_40459 | ATACATCGGATGATGATACC (SEQ ID NO: 49) |
| oligo | Knockout | | |
| CRISPR | MORC2 | GPP_31095 | ACATTAGAAGTACGCCTAGG (SEQ ID NO: 50) |
| oligo | Knockout | | |
| CRISPR | SETDB1 | GPP_26249 | AAGGAAAGAGTCTACTGTCG (SEQ ID NO: 51) |
| oligo | Knockout | | |
| CRISPR | C5orf30 | GPP_55433 | TGACACGGTCCTCCGCATCG (SEQ ID NO: 52) |
| oligo | Knockout | | |
| CRISPR | C5orf30 | GPP_55434 | TTAGCTCAGAAGTGCACAGG (SEQ ID NO: 53) |
| oligo | Knockout | | |
| CRISPR | MAP2K3 | GPP_14931 | CTTGGACAAGTTCTACCGGA (SEQ ID NO: 54) |
| oligo | Knockout | | |
| CRISPR | MAP2K3 | GPP_14932 | TTGGTGACCATCTCAGAACT (SEQ ID NO: 55) |
| oligo | Knockout | | |
| CRISPR | HAX1 | GPP_28138 | CCCCAACCAGCACCAGACTG (SEQ ID NO: 56) |
| oligo | Knockout | | |
| CRISPR | HAX1 | GPP_28137 | AAGACCCCCCCAAAGATCCT (SEQ ID NO: 57) |
| oligo | Knockout | | |
| CRISPR | RASA2 | GPP_15862 | AGGATCGACTTGTGGAACAA (SEQ ID NO: 58) |
| oligo | Knockout | | |
| CRISPR | RASA2 | GPP_15861 | AGATATCACACATTACAGTG (SEQ ID NO: 59) |
| oligo | Knockout | | |
| CRISPR | UBE3C | GPP_25625 | CATTCAGAAAGGGCTCGTAA (SEQ ID NO: 60) |
| oligo | Knockout | | |
| CRISPR | UBE3C | GPP_25627 | TGACTTTGGGTACCATCATG (SEQ ID NO: 61) |
| oligo | Knockout | | |
| CRISPR | FOXO3 | GPP_06436 | CCTGCCATATCAGTCAGCCG (SEQ ID NO: 62) |
| oligo | Knockout | | |
| CRISPR | FOXO3 | GPP_06434 | CAGAGTGAGCCGTTTGTCCG (SEQ ID NO: 63) |
| oligo | Knockout | | |
| CRISPR | TNFRSF10B | GPP_22999 | TGCAGCCGTAGTCTTGATTG (SEQ ID NO: 64) |
| oligo | Knockout | | |
| CRISPR | TNFRSF10B | GPP_23000 | TGTGCCGGAAGTGCCGCACA (SEQ ID NO: 65) |
| oligo | Knockout | | |
| CRISPR | FAT1 | GPP_06096 | CTACGACAGTCACTTCGATG (SEQ ID NO: 66) |
| oligo | Knockout | | |
| CRISPR | FAT1 | GPP_06095 | CTAACCGTTAGAGCTTCCGA (SEQ ID NO: 67) |
| oligo | Knockout | | |
| CRISPR | NLRX1 | GPP_49583 | AGGGCCTTTATACGCCACCA (SEQ ID NO: 68) |
| oligo | Knockout | | |
| CRISPR | NLRX1 | GPP_49586 | TTCTGGACTGGTGTTATGGG (SEQ ID NO: 69) |
| oligo | Knockout | | |
| CRISPR | GLYATL2 | GPP_65486 | ATTACCAGATCGTCATTACC (SEQ ID NO: 70) |
| oligo | Knockout | | |
| CRISPR | GLYATL2 | GPP_65488 | GAGCAAACTTTGCAGATCCA (SEQ ID NO: 71) |
| oligo | Knockout | | |
| CRISPR | CARD8 | GPP_31163 | AAAATGGGCAACGAGAAACC (SEQ ID NO: 72) |
| oligo | Knockout | | |
| CRISPR | CARD8 | GPP_31166 | GAAGAGCAGGAATCTTCAGA (SEQ ID NO: 73) |
| oligo | Knockout | | |
| CRISPR | C4orf29 | GPP_50826 | TCCTCCCATCACAAAAAGGT (SEQ ID NO: 74) |
| oligo | Knockout | | |
| CRISPR | C4orf29 | GPP_50824 | CAATTGAATCTGTTATTGCA (SEQ ID NO: 75) |
| oligo | Knockout | | |
| CRISPR | TTC12 | GPP_41176 | CTTACCTGGTCAGGTCAAGG (SEQ ID NO: 76) |
| oligo | Knockout | | |
| CRISPR | TTC12 | GPP_41175 | AGCTATCCTGCGCTACAGTG (SEQ ID NO: 77) |
| oligo | Knockout | | |
| CRISPR | BOD1L1 | GPP_66975 | TATGGTTTCCAATATCGACA (SEQ ID NO: 78) |
| oligo | Knockout | | |
| CRISPR | BOD1L1 | GPP_66974 | GGATCATACGATTCCCTCAG (SEQ ID NO: 79) |
| oligo | Knockout | | |
| CRISPR | RNF217 | GPP_62881 | AAAACACCAGACGAATTGGC (SEQ ID NO: 80) |
| oligo | Knockout | | |
| CRISPR | RNF217 | GPP_62882 | ATTTGCATCCAAATATACTG (SEQ ID NO: 81) |
| oligo | Knockout | | |
| CRISPR | PIK3CB | GPP_13974 | AAAGAGCACTTGGTAATCGG (SEQ ID NO: 82) |
| oligo | Knockout | | |
| CRISPR | PIK3CB | GPP_13976 | TGTAGCGTGGGTAAATACGA (SEQ ID NO: 83) |
| oligo | Knockout | | |
| CRISPR | C5orf46 | GPP_71359 | GACGACAAGCCAGACGACTC (SEQ ID NO: 84) |
| oligo | Knockout | | |
| CRISPR | C5orf46 | GPP_71357 | CCAGGAGGCTTAGGAATTTG (SEQ ID NO: 85) |
| oligo | Knockout | | |
| CRISPR | ZNRF1 | GPP_54539 | AGGTGGTTCAGCTCGCATAG (SEQ ID NO: 86) |
| oligo | Knockout | | |
| CRISPR | ZNRF1 | GPP_54542 | GTCCGGTAGTGCCCGAAATG (SEQ ID NO: 87) |
| oligo | Knockout | | |
| CRISPR | SPEN | GPP_31455 | AAGGAGCGTCTATGCAACCA (SEQ ID NO: 88) |
| oligo | Knockout | | |
| CRISPR | SPEN | GPP_31458 | GTTTGCCTACCTCGTGAACG (SEQ ID NO: 89) |
| oligo | Knockout | | |
| CRISPR | SMARCE1 | GPP_17777 | ACCAACAGCCGGGTCACGGT (SEQ ID NO: 90) |
| oligo | Knockout | | |
| CRISPR | SMARCE1 | GPP_17779 | TCGACAGAGACAATCTCGCA (SEQ ID NO: 91) |
| oligo | Knockout | | |
| CRISPR | MOB3A | GPP_59112 | GGGCGAGGACCTGAACGACT (SEQ ID NO: 92) |
| oligo | Knockout | | |
| CRISPR | MOB3A | GPP_59110 | CGAGGCGCAGATCAACAACG (SEQ ID NO: 93) |
| oligo | Knockout | | |
| CRISPR | ZNF609 | GPP_31610 | TGGTGGTAAATGTAACGTGG (SEQ ID NO: 94) |
| oligo | Knockout | | |
| CRISPR | ZNF609 | GPP_31608 | CTCTACAAGTCGAACTTTGG (SEQ ID NO: 95) |
| oligo | Knockout | | |

Samples were demultiplexed and 20 bp CRISPR sgRNA sequences were aligned to the Brunello reference index using Bowtie, allowing no mismatches. We restricted our analysis to genes with FPKM >1 in RPE cells. Read count data was analyzed to quantify knockout cell proportions with MAGeCK software v0.5.6 or v0.5.7 with the following key parameters: —norm-method control, —additional-rra-parameters ‘—permutation 10000 —min-percentage-goodsgrna 0.6’. Gene pvalues from MAGeCK were converted into Z scores and combined by Stouffer's method (Zₛ=Σᵢ₌₁ⁿZᵢ/\sqrt{n}); i=gene ID, n=total number of timepoints in which gene i was identified. We filtered this list by limiting the number of overlapping 95% confidence intervals among timepoints to fewer than 5. Gene knockouts with differential fitness effects on LINE-1(+) cells as compared to control were analyzed for overrepresentation of GO terms using Webgestalt. Individual GO categories were then analyzed in StringDB to generate network plots. To determine enrichment of genes encoding nuclear proteins, we used a Chi-square test following the null hypothesis that only 35.2% of genes should encode nuclear proteins based on the genetic composition of the Brunello library. Analysis of HUSH complex genes was pursued based on knowledge of the LINE-1 literature, as this complex is not annotated in current gene sets.

RNAseq

LINE-1 or luciferase was induced for 3 days with 1 μg/ml doxycycline and RNA was collected with the Quick-RNA Microprep kit (Zymo). Libraries were prepared with the TruSeq stranded mRNA library preparation kit (Illumina). Paired-end 150 bp reads were obtained on an Illumina HiSeq4000. Demultiplexed libraries were aligned to hg38 using STAR v2.4.5. Quantification and differential expression analysis was performed using the HTseq and DESeq2 packages in R. For gene set enrichment analysis, we isolated genes with |log2Fold-Change|>1 and p-adjusted <1.8e-6 and used GSEA software v2.0 from the Broad Institute against Hallmark, Biocarta, KEGG, and Reactome genesets v6.2. We used loge Fold-Change values to perform a pre-ranked analysis.

Western Blots for LINE-1 Analysis

Cells were lysed in RIPA buffer with protease/phosphatase inhibitor (cat#5872, Cell Signaling Technology, Danvers, Mass.) or Laemmli Sample Buffer (cat#1610747, Biorad, Hercules, Calif.) by sonication. PAGE was carried out with manufacturer-recommended buffers on 4-20% or 7.5% Mini TGX Gels (Biorad), NuPAGE 4-12% BisTris gels, or NuPAGE 3-8% Tris-Acetate gels (Thermo). Semi-dry transfers were carried out for Biorad gels or NuPAGE BisTris gels at 2.5 A for 5-15 minutes using the Trans-Blot-Turbo (Biorad). Wet transfers were carried out for Tris-Acetate gels at 30V overnight. All blocking was performed with Odyssey Blocking Buffer (Licor). Primary antibodies were incubated with membranes overnight at 4C, then infrared-conjugated secondaries (Licor) were added 1:10,000 and imaged on a Licor Odyssey Scanner. Quantifications were carried out using Image Studio v4.0. Blots were stripped with Reblot Plus Strong Solution (Millipore Sigma).

Cloning

Plasmids used in this study are listed in Table 2. The mammalian expression vector pCEP4 (Invitrogen) was modified to possess a 2^{nd} or 3^{rd} Generation Tet-inducible promoter (ClonTech) by Gibson assembly. LINE-1 sequences were inserted into the vector backbone by Gibson assembly with PCR amplicons of endogenous LINE-1 sequence (LINE-1 RP) or ORFeus codon-optimized sequence. Control pCEP4 vectors encoded either eGFP or lacked expression inserts. LINE-1 point mutant constructs were also created by amplification and Gibson assembly. For sleeping-beauty integrated LINE-1, ORFeus codon-optimized LINE-1 was cloned into the donor vector pSBtet-RN or pSBtet-GN by Gibson assembly. Briefly, pSBtet-RN or GN was digested with SfiI and DraIII, gel purified and assembled with PCR-amplified LINE-1 (primers SB-ORFeus-5 and SB-ORFeus-3 in Supplementary Table 6) using the HiFi 2X Assembly Master Mix (NEB, Ipswich, Mass.).

TABLE 2

| Plasmid | | | | | | |
|---|---|---|---|---|---|---|
| ID | plasmid name | Purpose | Source | Insert | Promoter | Marker(s) |
| DA007 | pDA007 | ORFeus | Burns | ORFeus-Hs, | Tet, 2nd | puromycin |
| | | expression, WT | Lab | ORF2-3xFlag | | |
| DA019 | pDA019 | control | Burns | Multiple | Tet, 2nd | puromycin |
| | | expression | Lab | Cloning Site | | |
| | | vector | | | | |
| DA025 | pDA025 | ORFeus | Burns | ORFeus | Tet, 2nd | puromycin |
| | | expression, | Lab | H230A En, | | |
| | | mutant | | ORF2-3xFlag | | |
| DA027 | pDA027 | ORFeus | Burns | ORFeus | Tet, 2nd | puromycin |
| | | expression, | Lab | H230A/D702Y, | | |
| | | mutant | | ORF2-3xFlag | | |
| DA034 | pDA034 | ORFeus | Burns | ORFeus | Tet, 2nd | puromycin |
| | | expression, | Lab | D702Y RT, | | |
| | | mutant | | ORF2-3xFlag | | |
| DA055 | pDA055 | ORFeus | Burns | ORFeus-Hs, | Tet, 3rd | hygromycin |
| | | expression, WT | Lab | ORF2-3xFlag | | |
| DA056 | pDA056 | ORFeus | Burns | ORFeus-Hs | Tet, 3rd | blasticidin |
| | | expression, WT | Lab | | | |
| DA064 | psPAX2 | lentivirus | AJ | | | |
| | | packaging | Holland | | | |
| DA065 | pMD.G | lentivirus | AJ | | | |
| | | packaging | Holland | | | |
| DA077 | pDA077 | L1RP | Burns | L1RP | CMV | neomycin |
| | | expression | Lab | | | |
| DA079 | pOT_p53shRNA_Lentivirus_TagRFP_T | TP53 shRNA | AJ | p53 shRNA | H1 | TagRFP |
| | | lentivirus | Holland | | | |
| DA081 | pSicoR_mCh_empty | control for TP53 | Addgene | empty | H1 | mCherry |
| | | shRNA lenti | | | | |
| DA083 | pDA083 | eGFP | Burns | eGFP | CMV | neomycin |
| | | expression | Lab | | | |
| DA090 | pCMV(CAT)T7-SB100 | generation of | Addgene | sleeping | CMV | |
| | | Tet-On RPE | | beauty | | |
| DA091 | pSBtet-RN | generation of | Addgene | Luciferase | Tet, 2nd | G418, RFP |
| | | Tet-On RPE | | | | |
| DA093 | pDA093 | generation of | Burns | ORFeus | Tet, 2nd | G418, RFP |
| | | Tet-On RPE | Lab | | | |
| DA094 | pSBtet-GN | generation of | Burns | Luciferase | Tet, 2nd | G418, |
| | | Tet-On RPE | Lab | | | eGFP |
| DA095 | pDA095 | generation of | Burns | ORFeus | Tet, 2nd | G418, |
| | | Tet-On RPE | Lab | | | eGFP |
| DA097 | pLentiGuide-Puro | generation of | Addgene | | U6 | puromycin |
| | | CRISPR KO | | | | |
| | | cells | | | | |
| JM111 | pJM111 | L1RP | Kazazian | L1RP | CMV | puromycin |
| | | retrotransposition | Lab | (inactive) | | |
| | | reporter, | | GFP-AI | | |
| | | negative control | | | | |
| MT525 | pMT525 | L1RP | Boeke | L1RP | CMV | puromycin |
| | | retrotransposition | Lab | GFP-AI | | |
| | | reporter | | | | |

Single-Gene CRISPR Knockout Cell Generation

To validate screen hits, we cloned 20 bp CRISPR sgRNAs into the pLentiGuide-Puro vector digested with BstBI restriction enzyme as previously described and then packaged the plasmids into lentivirus. We selected sgRNAs that had enriched in the screens. See Table 1 for sgRNA sequences. Cells were incubated with lentiviral supernatants supplemented with 10 μg/ml polybrene for 24 hours, then selected with puromycin for 1 week, and then used in downstream assays, including clonogenic assays and western blots.

Transfection

293 and Hela cells were transfected with Fugene HD reagent (Promega, Madison, Wis.) following standard protocols. RPE cells were transfected using midi- or maxi-prepped plasmid DNA with Viafect reagent (Promega, Madison, Wis.) at a DNA:Viafect ratio of 1:3. See supplemental Table 2 for plasmids.

Lentivirus Packaging

293FT cells were transfected with Fugene HD (Promega, Madison, Wis.) following the manufacturer's recommendations. Insert vector was added to packaging plasmids pMD.G and psVAX2 at a ratio of 3:4:1 by mass. Media was changed after 24 hours and 48 hours later viral supernatants were collected and filtered through 0.45 um filters. For screen libraries, complex lentivirus pools were packaged by a similar method by Applied Biological Materials Inc. (Richmond, BC, Canada).

Retrotransposition Reporter Assay

2e5 RPE cells were transfected with 2 ug LINE-1 reporter plasmids (MT525, JM111) or 2 ug eGFP plasmid and selected with 1 μg/ml puromycin for 12 days. Cells were trypsinized and resuspended in cytometry buffer (Hanks Balanced Salt Solution, no phenol red, 1% FBS, 1 mM EDTA) at a concentration of ˜1e6 cells/mL, then analyzed on a BD Accuri C6 Flow Cytometer. Singlets were gated on SSC-A/SSC-H and FSC-A/FSC-H, then eGFP thresholds were set such that untransfected cells showed 0.1% eGFP+ cells. We normalized the % GFP+ cells in experimental groups to % GFP+ in eGFP-controls.

Nucleoside Reverse Transcriptase Inhibitor Treatments for qRT-PCR

250,000 Tet-On TP53^{KD} cells expressing Luciferase or LINE-1 were plated in T25 flasks with 1 ng/mL doxycycline added and treated with 5 μM zalcitabine (ddC) or 5 μM didanosine (ddI) for 72 hours. Cells were lysed and RNA were extracted using Quick-RNA MicroPrep kit (Zymo Research).

qRT-PCR

cDNA was generated using the iScript kit (Biorad, Hercules, Calif.) following RNA extraction using the Quick-RNA Microprep kit (Zymo). Primers were designed using Primer3 and tested against cDNA to ensure single bands were generated in the PCR. Real-time PCR was performed for 40 cycles (98C×15 seconds, 60C×30 seconds) using SSOAdvanced 2X Master mix (Biorad) on the MyIQ cycler (Biorad). Fold-change expression was determined by the 2^{-ΔΔCt} method. See Table 1 for primer sequences.

Immunofluorescence Imaging

HEK293T cells were transfected with doxycycline-inducible LINE-1 plasmid (pDA055) and stably-selected with hygromycin for 2 weeks. 5,000 cells were plated in a black 96-well, glass-bottom plate (Corning, cat#3603), treated with doxycycline (0 to 5,000 ng/mL, 24 hours), fixed (3% paraformaldehyde, 10 minutes), permeabilized (0.5% Triton X-100/PBS-Glycine, 3 minutes), and blocked (1% BSA/PBS-Glycine, 30 minutes). Primary antibodies: ORF1p and FLAG, both at 1:500 dilution; Hoechst 33342 (1:50 dilution, Sigma) for nuclear DNA; and HCS CellMask deep red cytoplasmic stain (1:20000 dilution, Invitrogen). Secondary antibodies: anti-rabbit Alexa Fluor 488 (1:200, Invitrogen) and anti-mouse Alexa Fluor 568 (1:200, Invitrogen). Imaging performed with a TE300 epifluorescent microscope (Nikon, Melville, N.Y.) with a motorized stage and excitation/emission filters (Prior). Images acquired with a DS-QiMc camera at low magnification (20X Plan Fluor lens; 0.285 μm/pixel, Nikon) using Nikon Elements software (Nikon). Twenty-five images were acquired per sample in a 5×5 grid (1.88 mm²). Images analyzed using a custom MATLAB software to segment single cells using the HCS CellMask stain and nuclei using Hoechst 33342. Accurate cell segmentation was manually verified to create a subset of 100 single cells in which ORF1p and ORF2p signal strengths were measured as the total intensity within each segmented cell for each fluorescence channel.

Nuclear Foci Quantification

We used either Tet-On TP53^{KD} cells expressing Luciferase or LINE-1 or HeLa cells transfected with doxycycline-inducible LINE-1 plasmids (pDA007, pDA025, pDA027, pDA033, pDA019) and stably-selected with puromycin for 1-2 weeks. Positive controls were treated with either 6 mM hydroxyurea for 4 hours or 200 ng/mL doxorubicin for 2 hours. 100,000 cells were plated on cover slips and treated with 1000 ng/ml doxycycline for 72 hours. EdU was added for 2 hours and cells were pre-treated with 0.5% Triton X-100 for 5 min, fixed with 3.7% paraformaldehyde for 10 minutes, then permeabilized with 0.5% NP-40 for 10 minutes. EdU Click-iT reaction (ThermoFisher) was performed following manufacturer's instructions. Slides were blocked (1% BSA/PBS-Glycine, 30 minutes) and incubated with polyclonal rabbit FANCD2 (1:1000, Novus Biologicals), rabbit 53BP1 (1:500, Novus Biologicals), or mouse γH2A.X (1:1000, Millipore) for 1 hour at room temperature and then anti-rabbit Alexa Fluor 488 for FANCD2 (1:200, ThermoFisher) and anti-rabbit Alexa Fluor 488 (1:2000, ThermoFisher) and anti-mouse Alexa Fluor 555 (1:2000, ThermoFisher) for 53BP1 and γH2A.X, respectively. Slides were imaged at low magnification with the same equipment as described above with key methodological differences. Randomly-selected nuclei (>200 per sample) were imaged at high magnification. Foci were quantified using a previously published method in MATLAB. We categorized cells as S phase (EdU+) or G1/G2 phase (EdU−) and excluded cells with sub-2n DNA content (dying cells). We compared foci counts using unpaired two-sided T-tests.

Transposon Insertion Sequencing (TIP-seq) and PCR Validations

Tissues for TIP-seq were acquired as flash-frozen de-identified surgical specimens. Small sections of each frozen tissue sample were isolated and TIP-seq was performed as previously described. Briefly, 10 μg of DNA was digested with Asel, BspHI, BstYI, HindIII, NcoI, or PstI (NEB). Vectorettes matching the sticky ends were ligated and touchdown PCR was run with an L1PA1-specific primer (5′-AGATATACCTAATGCTAGATGACACA-3′) (SEQ ID NO: x) and ExTaq HS polymerase (Takara Bio; Shiga, Japan). We combined six PCR reactions for each sample and purified the DNA for sequencing library preparation shearing amplicons to an average size of 300 bp. We then performed end-repair, dA-tailing and index-specific adaptor ligation steps according to Illumina's TruSeq DNA Sample Prep v4 kit protocol (Illumina; San Diego, Calif.). Using 2% Size-Select E-gels (Life Technologies; Carlsbad, Calif.), we size-selected our adaptor-ligated DNA at approximately 450 bp before performing a final PCR amplification. After purifying the PCR amplified libraries, we submitted them for quality control and Illumina HiSeq4000 150-bp paired-end sequencing at the NYU Genome Technology Center. Insertions were called using TIPseqHunterV2 after alignments to hg19. We validated insertions by designing PCR primers with Primer3 and amplifying the insertions. We performed genotyping PCR reactions using 1 ng input DNA of both flash-frozen surgical specimens and DNA obtained from formalin-fixed paraffin embedded tissue using the QIAamp DNA FFPE Tissue Kit (Qiagen).

Quantification and Statistical Analysis

In CRISPR KO screens and RNAseq analyses, statistical testing was included in the software packages (MAGeCK and DESeq2, WebGestalt, GSEA, StringDB). For all other analyses, appropriate statistical tests were performed using R, which is indicated in figure legends. Tests were typically unpaired and included both one- and two-sided T tests or ANOVA depending on the a priori hypothesis.

Detection of L1 ORF Peptides in CPTAC Data

The CPTAC discovery breast and ovarian mass spectrometry data was used (available at the CPTAC Data Portal: cptac-data-portal.georgetown.edu/cptac/s/S015 and cptac-data-portal.georgetown.edu/cptac/s/S020, respectively). For the detection of ORF1p and ORF2p peptides, we constructed a protein sequence collection that, in addition to human proteins from Ensembl, also included high confidence LINE-1 proteins from L1Base2: 292 ORF1p/ORF2p sequences translated from full-length intact LINE-1 and 107 ORF2p translated from ORF2 intact LINE-1 elements in human, and 89 LINE-1 ORF1p/ORF2p translated from ancestor consensus sequences. In addition, we also included a list of contaminant proteins from the common Repository of Adventitious Proteins (cRAP). We used the X! Tandem (thegpm.org/tandem/) search engine with the curated databases and the same search parameters as in. In-house scripts were used to parse the X! Tandem outputs to filter for high-quality Peptide-Spectrum Matches (PSMs). Only PSMs that meet the following criteria were retained: the fraction of the intensity of peaks that matched the sequence >40%, the gaps in the fragmentation were not larger than 3 amino acids, the peptide length >=7 and the e-value <=0.01. We also eliminated PSMs that match to more than one gene. In order to select a set of reliable peptides from ORF1p, we performed a pair-wise comparison of the peptide quantities only kept the peptides that formed a set that had a Spearman correlation of 0.6 with each other.

Purification of ORF2 Proteins

pGC6, expressing ORF2 endonuclease, is tagged with N-HIS6-TEV. We expressed overnight in bacteria at 16° C., then shifted temperature and induced with IPTG. We froze cell pellets then purified on a nickel column in standard conditions. We cleaved the tag with TEV protease overnight, then performed gel filtration to clean up the untagged protein. pLD75, expressing ORF2 reverse transcriptase tagged with His-MBP, was expressed similarly, purified on a nickel column, then with cation exchange (HiTrap SP FF; GE Healthcare Life Sciences). pLD561, expressing full-length ORF2p-3× Flag, was expressed as a 15 L culture in suspension HEK-293T_{LD}. We lysed cells with a microfluidizer in 500 mM NaCl buffer with 1% (v/v) Triton X-100, then performed Flag IP using Dynabeads (Thermo Fisher Scientific) coupled to anti-Flag M2 (Millipore Sigma) followed by 3× Flag elution.

Generation of Monoclonal Antibodies

Rabbit monoclonal antibodies were developed with Abcam (Cambridge, Mass.). For EN-targeting antibodies, rabbits were immunized and boosted with EN, screened by ELISA for EN affinity, and then hybridoma supernatants were tested against ORF2-3× Flag by ELISA. For RT-targeting antibodies, rabbits were immunized with MBP-tagged RT, boosted with SUMO-tagged RT, then screened by ELISA with MBP-RT and counter-screened with MBP and SUMO to eliminate clones that were specific for MBP or SUMO.

ORF2p Induction in HEK-293T_{LD} Cells

Plasmid DNA was miniprepped using the Zyppy Miniprep Plasmid DNA kit (Zymo, Irvine, Calif.) or PureLink HiPure Plasmid Midiprep Kit (Thermo Fisher, Waltham, Mass.). These were transfected into Tet-On HEK-293T_{LD} cells by incubating 3 μg plasmid DNA with 9 μL Fugene HD (Promega, Madison, Wis.) in 100 μL Optimem for 15 min, then adding dropwise to 6-well plates containing 500,000 cells per well. 1 μg/ml doxycycline was added at the time of transfection and cells were then used for immunoprecipitation, immunofluorescence, immunohistochemistry, or western blot assays 24 hr later.

ORF1p and ORF2p Immunohistochemistry

For cells, HEK-293T_{LD} cells expressing a plasmid encoding ORF2-3× Flag were admixed with untransfected HEK-293T_{LD} and pelleted, fixed in 10% formalin for 24 hr, then processed into paraffin-embedded blocks. For human tissue samples, de-identified paraffin-embedded blocks were obtained from the Pathology Department at Massachusetts General Hospital. Formalin-fixed paraffin embedded tissues were sectioned at 5 μm onto glass slides, heated to 65° C. for 20 min, and then rehydrated by serial washes in xylene, ethanol (100%/90%/75%), and water. IHC was performed with the DAKO EnVision+ System-HRP kit (cat#K4006, Agilent, Santa Clara, Calif.). Antigen retrieval was performed using Target Retrieval Solution for 20 minutes at >90° C., then slides were blocked with peroxidase block and then 2% (w/v) BSA in PBS. Primary antibody incubation with ORF1p was performed at 1:5000 for 1 hr at room temperature and with ORF2p MT49 overnight at 4° C. at a final concentration of 10 μg/ml, and secondary HRP mouse polymer secondaries were used to label primary antibody with chromogen upon DAB addition. Hematoxylin was used as a nuclear counterstain. Slides were then dehydrated in serial washes and coverslips were placed. Scoring was performed by a trained pathologist.

Immunofluorescence (IF)

HEK-293T_{LD} cells expressing a plasmid encoding ORF2-3× Flag were admixed with untransfected HEK-293T_{LD} and pelleted, fixed in 10% formalin for 24 hr, then processed into paraffin-embedded blocks. IF was performed on 5 μM sections. Slides were processed as for IHC but using AlexaFlour-conjugated secondary antibodies (anti-rabbit 488 and anti-mouse 555). Imaging was performed on a Zeiss Confocal Microscope.

Antibody Epitope Mapping

A library of peptide based epitope mimics was synthesized using solid-phase Fmoc synthesis. An amino functionalized polypropylene support was obtained by grafting with a proprietary hydrophilic polymer formulation, followed by reaction with t-butyloxycarbonyl-hexamethylenediamine (BocHMDA) using dicyclohexylcarbodiimide (DCC) with N-hydroxybenzotriazole (HOBt) and subsequent cleavage of the Boc-groups using trifluoroacetic acid (TFA). Standard Fmoc-peptide synthesis was used to synthesize peptides on the amino-functionalized solid support by custom modified JANUS liquid handling stations (Perkin Elmer). The binding of antibody to each of the synthesized peptides was tested in a pepscan-based ELISA. The peptide arrays were incubated with primary antibody solution (overnight at 4° C.). After washing, the peptide arrays were incubated with a 1:1000 dilution of anti-rabbit IgG HRP conjugate (DAKO) for 1 hr at 25° C. After washing, the peroxidase substrate 2,2′-azino-di-3-ethylbenzthiazoline sulfonate (ABTS) and 20 μl/ml of 3 percent H₂O₂ were added. After 1 hr, the color development was measured with a charge coupled device (CCD)—camera and an image processing system. Epitope targets were read as the largest contiguous stretch of amino acids shared by all peptides recognized by the primary antibodies.

Peptide Blocking Experiments

Blocking peptides were chosen to span two extra amino acids and were N-terminally acetylated and C-terminally amidated. Peptides were resuspended in acetic acid or ammonium acetate depending on their charge characteristics. To pre-block antibodies, 10, 100, or 1000-times excess peptide by weight was incubated with primary antibody mixture on a rotating wheel at 4° C. overnight. The next day, pre-blocked or no-block antibodies were conjugated to protein G dynabeads for 15 min at room temperature, then ORF2p-3× Flag HEK-293T_{LD} lysate was added for 1 hr at room temperature. The remaining protocol is the same as described above for immunoprecipitation.

Phage Immunoprecipitation and DNA Sequencing

The PhIP-Seq assay was described previously. Approximately 100 ng of each mAb was added to the combined T7 bacteriophage human peptidome library (unique genome and repetitive element sublibrary addition, 1×10⁵ plaque forming units for each phage clone in each library) and incubated with rotation overnight at 4° C. in deep 96-well plates in 1 mL total volume of phosphate-buffered saline. Negative controls for data normalization included eight mock immunoprecipitation reactions on each plate. mAb-phage complexes were captured by magnetic beads (20 μL of protein A-coated and 20 μL of protein G-coated, catalog numbers 10002D and 10004D, Invitrogen, Carlsbad, Calif.) for 4 hours at 4° C. with rotation and processed using the Agilent Bravo liquid handling system (Agilent Technologies, Santa Clara, Calif.). Beads were washed twice with 0.1% NP-40 in Tris-buffered saline (50 mM Tris-HCl with 150 mM NaCl, pH 7.5), resuspended in 20 μL of a Herculase II-containing PCR mix (catalog number 600679, Agilent Technologies), and ran for 20 PCR cycles followed by a second 20-cycle PCR using 2 uL of the initial PCR products to add barcodes and P5/P7 Illumina sequencing adapters. Pooled PCR products were sequenced using an Illumina HiSeq 2500 (Illumina, San Diego, Calif.) in rapid mode (50 cycles, single end reads). Data were normalized and analyzed using a z-scores algorithm according to Yuan et. al.

Cloning to Generate ORF2p M990 Variant

A plasmid with full-length codon-optimized L1 (pMT491) was digested with Notl-AscI, blunted with T4 polynucleotide kinase, and ligated with T4 DNA ligase to generate a doxycycline-inducible ORF2-3× Flag expression vector (pDA033). To generate the M990 mutant (Plasmid pDA101), we performed a 3-fragment multichange isothermal assembly using BsrGI/BstZ17I-digested pDA033 as the backbone (fragment 3), and PCR-amplified fragments containing the V990M mutant in the overlapping sequence. PCR fragments were amplified from pDA033 with Q5 polymerase (NEB) and assembly was performed with the HiFi Assembly Master Mix (NEB). Individual clones were screened for M990 mutations by Sanger sequencing. Fragment 1 was generated with primers 5′-TGAGCGGCTACAAGATCAACGTG-3′ (SEQ ID NO: 96) and 5′-GCTCATGAAGTCCTTGCCCATGCCGATGTCCTGGATGGTG-3′ (SEQ ID NO: 97). Fragment 2 was generated with primers 5′-CACCATCCAGGACATCGGCATGGGCAAGGACTTCATGAGC-3′ (SEQ ID NO: 98) and 5′-ACATGTGCACATTGTGCAGGT-3′ (SEQ ID NO: 99).

Immunoprecipitation

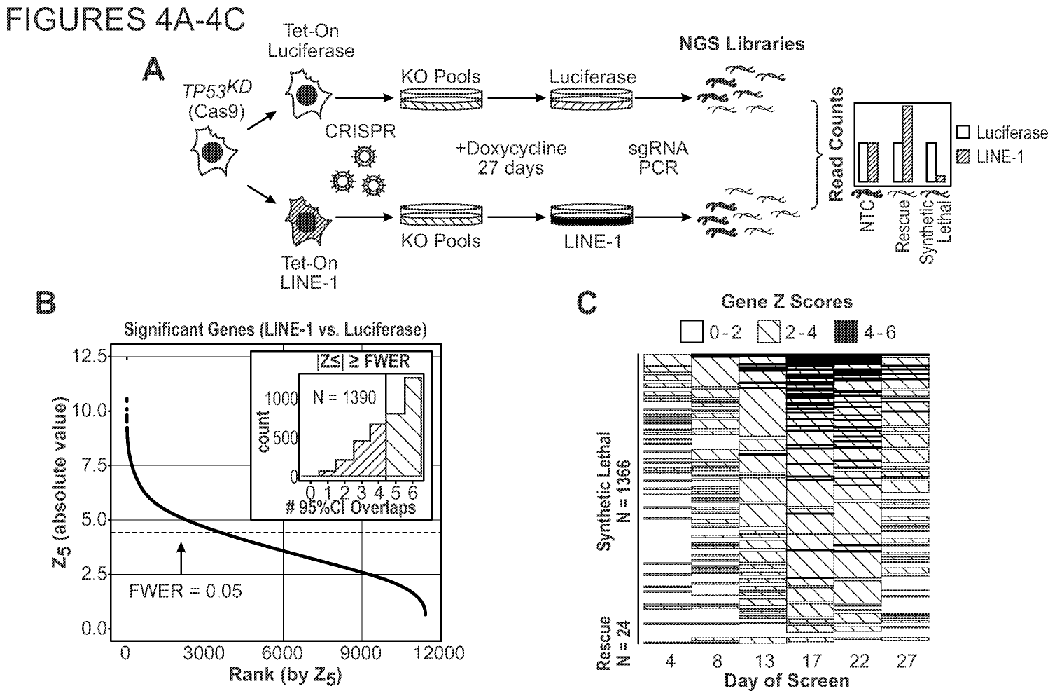

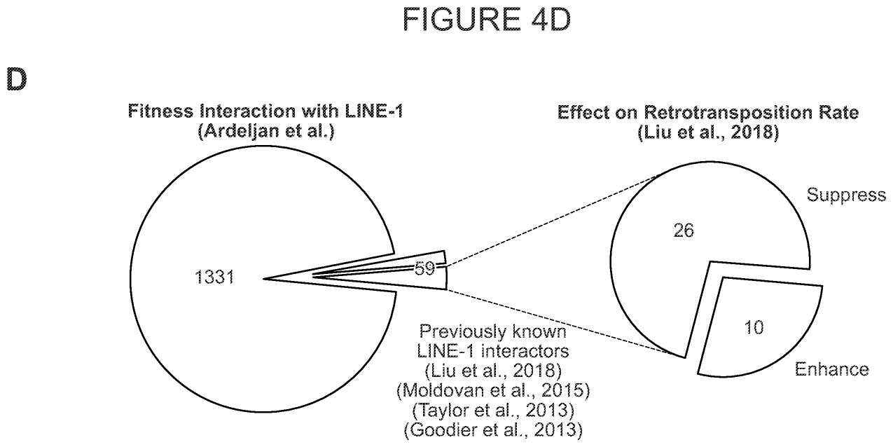

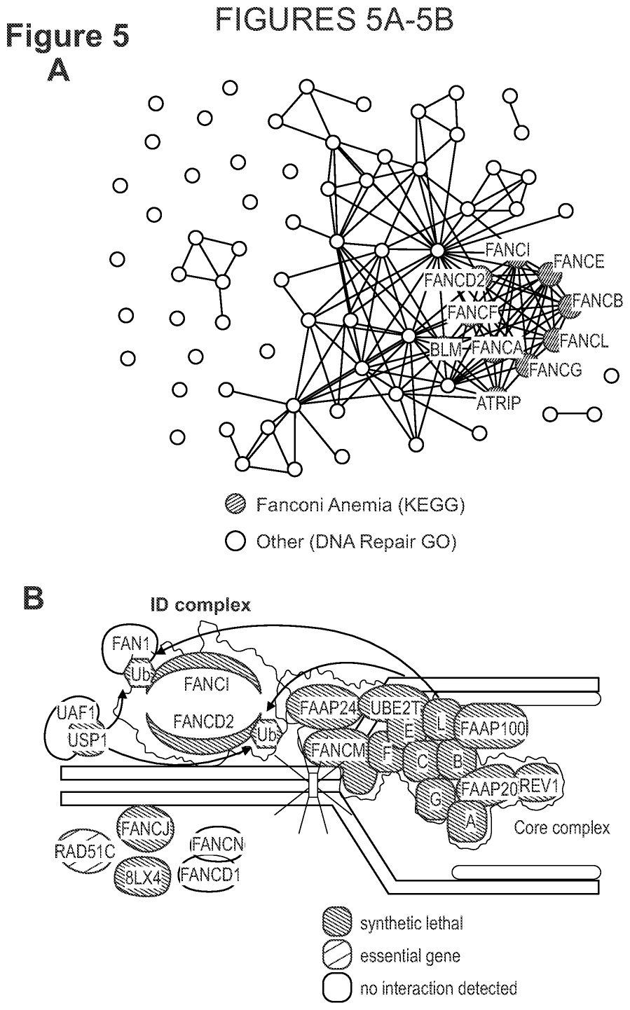

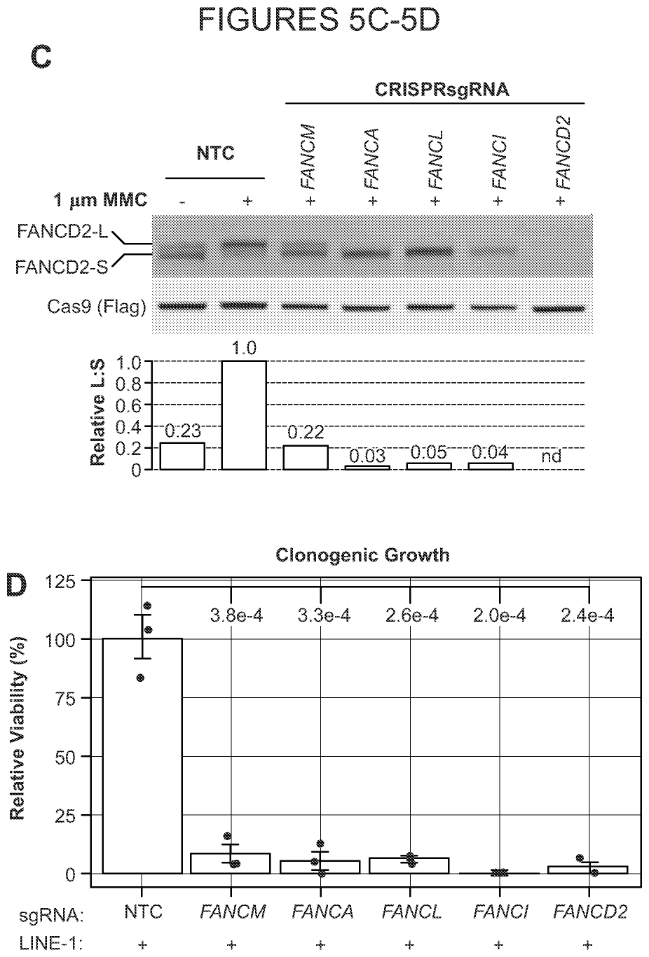

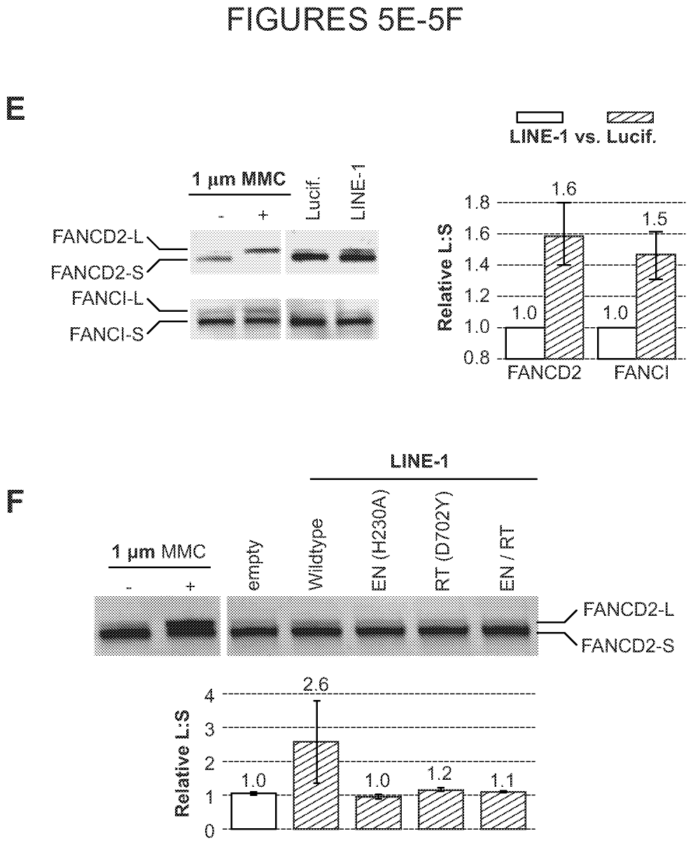

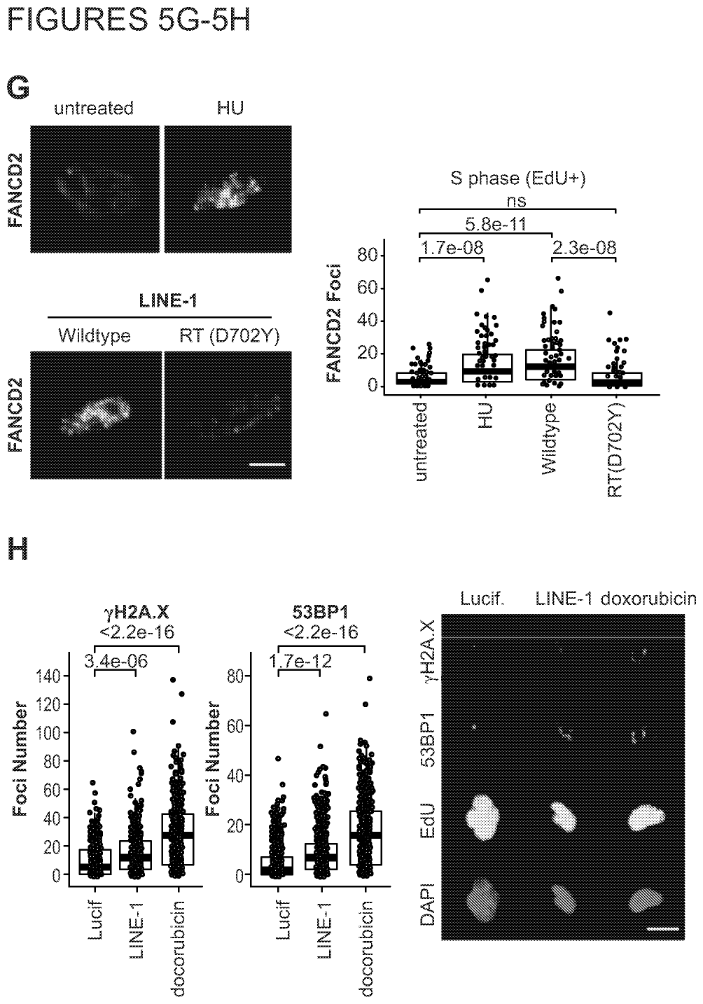

For FIGS. 4, 5, handling of cryomilled HEK-293TLD cells ectopically expressing L1 from pLD401 and pMT302 was previously described [Cell 2013, 155(5):1034-1048, Methods Mol Biol 2016, 1400:311-338]. Patient samples were milled and extracted similarly, as previously described [J Vis Exp 2016(118)]. Protein extraction solution: 20 mM HEPES pH 7.4, 500 mM NaCl, 1% (v/v) Triton X-100, 1× Roche Complete EDTA-free protease inhibitors. Tumor A was extracted in a separate instance in the same solution with the addition of Promega recombinant RNasin at 1:50 (v:v).

For patient samples subjected to LFQ-MS we used the following parameters: 200 mg-scale, 10 μl of anti-ORF1p (Millipore Sigma #MABC1152) and mouse IgG (Millipore Sigma #I5381) affinity medium were used per 200 mg-scale affinity capture. In addition to the mouse IgG mock affinity capture control, for tumors B and C, we carried out an additional mock affinity capture using the anti-ORF1p antibody and extracts from matched normal tissue, resected at the time the CRC was removed from the patient. Affinity media and clarified extracts were incubated for 1 hr at 4° C., washed three times with extraction solution, and eluted with NuPage sample buffer (Thermo Fisher Scientific #NP0007) at 70° C. After SDS-PAGE (Thermo Fisher Scientific: 1 mm, 4-12% Bis-Tris NuPAGE system), samples were analyzed by general protein staining, western blotting, and/or MS as described in the main text. Samples destined for MS were reduced (DTT) and alkylated (iodoacetamide) prior to electrophoresis. In a second instance, tumor A affinity isolations were conducted at a 100 mg-scale using 15 μl of anti-ORF1p and mouse IgG medium, were extracted and washed (3×250 μl washes as opposed to 1 ml) in the presence of 1:50 RNasin (not previously included), and 1× protease inhibitors (normally only present during extraction); approximately ⅔ the standard sonication energy was applied (the standard is 15-20 J per 100 mg-scale in a 25% (w:v) extract). In all cases, representative SDS-PAGE lanes are displayed in FIG. 4. Unless otherwise stated, all panels displayed have been ‘auto tone’ calibrated, respectively, in Adobe Photoshop to maximize the visual contrast across the detected signal range.

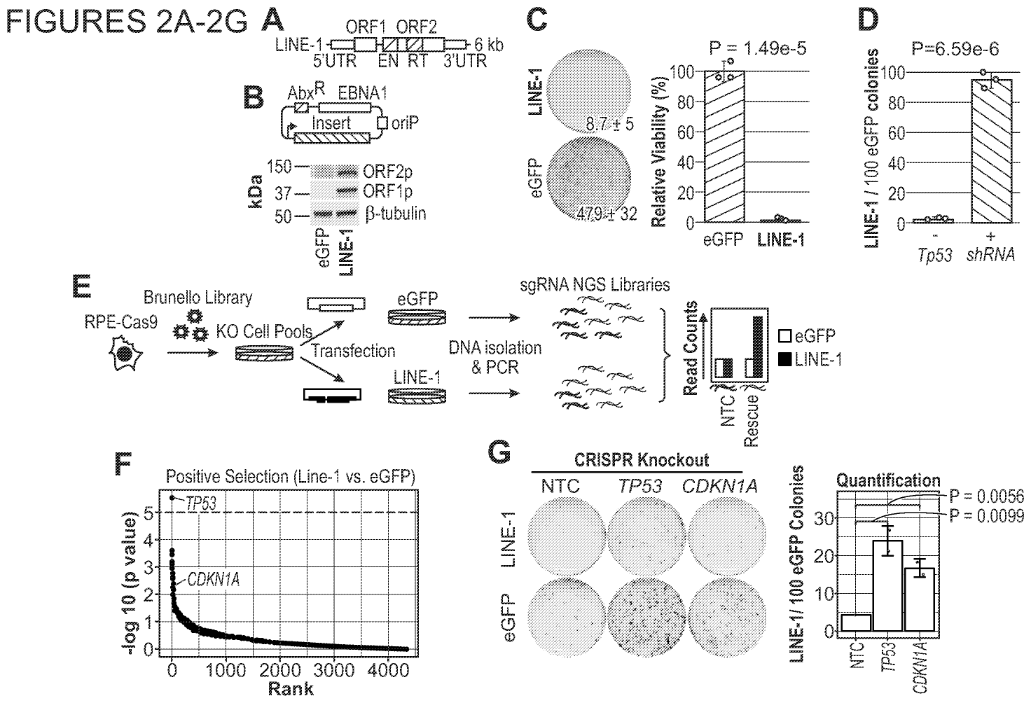

For FIGS. 2 and 3: HEK-293TLD cells transfected with a plasmid encoding ORF2-3× Flag were lysed by sonication in extraction buffer (50 mM NaCl, 20 mM HEPES pH 7.4, 1% Triton, 1 mM EDTA). 1 μg of each antibody was conjugated to 25 μL of Protein G Dynabeads for 15 minutes at room temperature, then washed with TBST. IP was carried out for 1 hour at room temperature on a rotating wheel with protein lysates diluted by TBST. For peptide blocks, antibodies were pre-incubated with peptide, conjugated to Dynabeads for 15 minutes, and lysate was added for 1 hour at room temperature. After IP, samples were washed in extraction buffer, then eluted from the Dynabeads by heating in LDS (Thermo) at 70° C. for 10 minutes. Supernatants were then run on Mini TGX gels (Biorad) for western blot with anti-Flag (Sigma) antibody.

Western Blotting

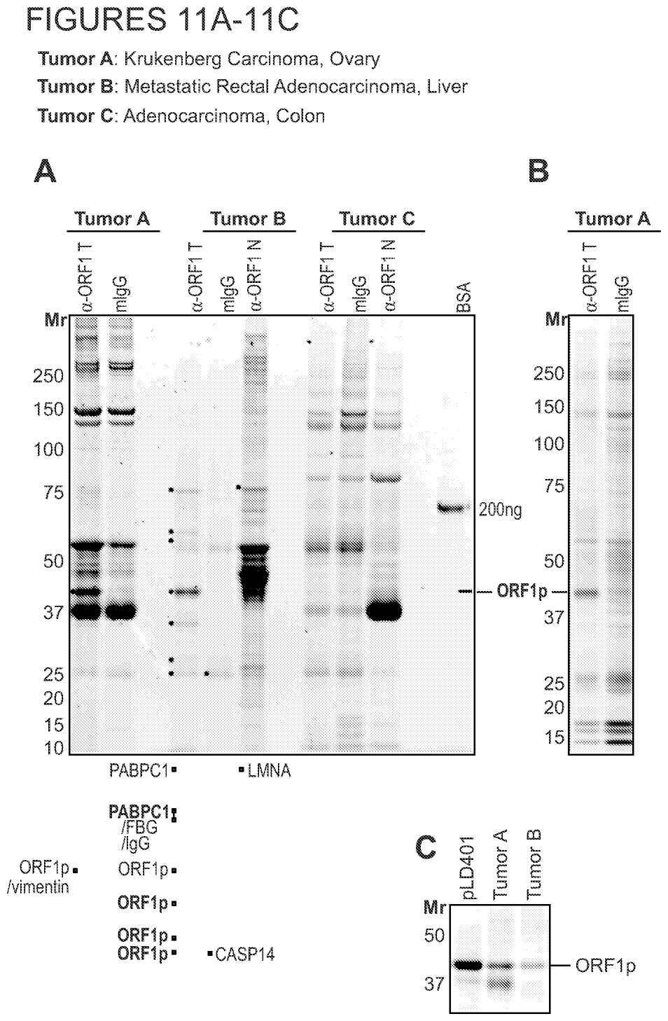

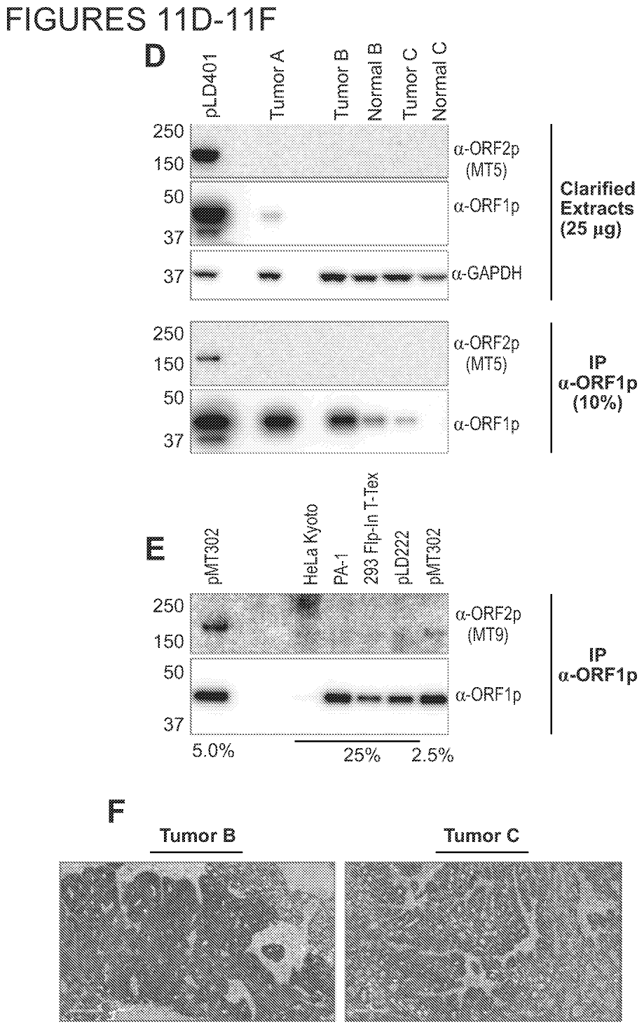

For western blots displayed in FIG. 11 the following parameters were used: wet transfer (1% [w/v] SDS/20% [v/v] methanol in transfer buffer) for 90 min/70 V/4° C., PVDF membrane (0.45 μm), HRP-conjugated secondary antibodies (see below), and chemiluminescent HRP detection (substrate: Millipore Sigma #WBLUF0100). Blocking was done overnight at 4° C. using 5% (w/v) nonfat dry milk in TBST (20 mM Tris-Cl, 137 mM NaCl, 0.1% Tween 20), pH 7.6. Primary antibodies were applied overnight at 4° C. in 5% (w/v) BSA in TBST, pH 7.6. Secondary antibodies were applied for 2 hr at room temperature in 5% (w/v) BSA in TBST, pH 7.6. Where appropriate, total protein quantities were estimated using a commercial Bradford reagent. An ImageQuant LAS-4000 system (GE Healthcare) was used for blot imaging on the high sensitivity setting with incremental image capture. ECL signal capture times displayed varied with target from ˜1-5 min and were free of pixel saturation in any signal displayed in the figures. Anti-ORF1p (Millipore Sigma #MABC1152) was used at 0.4 μg/ml; anti-ORF2p (this study) clone MT5 was used at 0.13 μg/ml and clone MT9 was used at 0.71 μg/ml; anti-GAPDH (Cell Signaling #2118) was used at 0.02 μg/ml. Secondary antibodies: anti-mouse HRP conjugate (GE Lifesciences #NV931) and anti-rabbit HRP conjugate (GE Lifesciences #NV934) were used at 1:10,000. All panels displayed have been ‘auto tone’ calibrated, respectively, in Adobe Photoshop to maximize the visual contrast across the detected signal range.

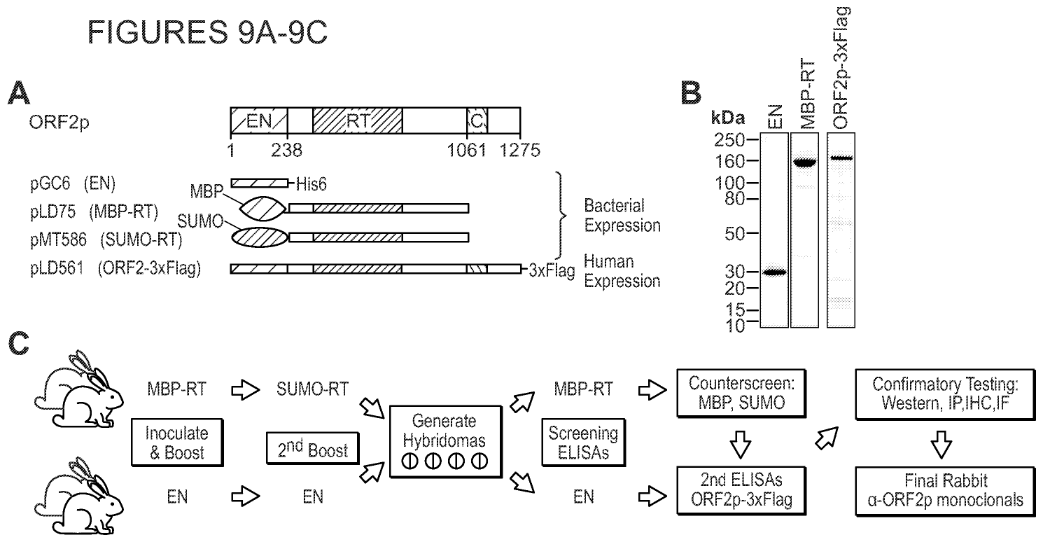

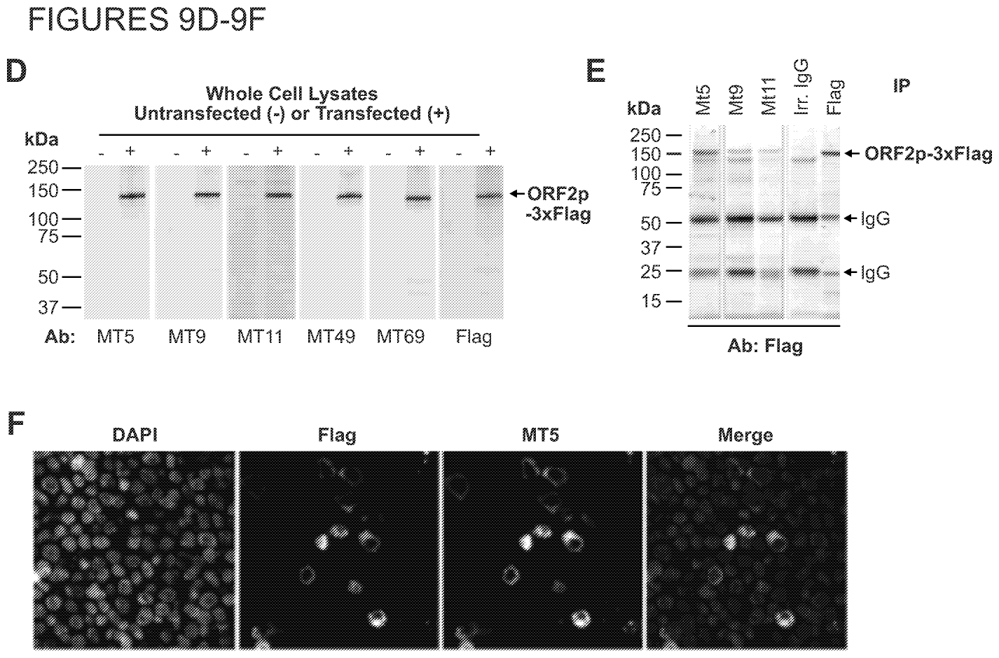

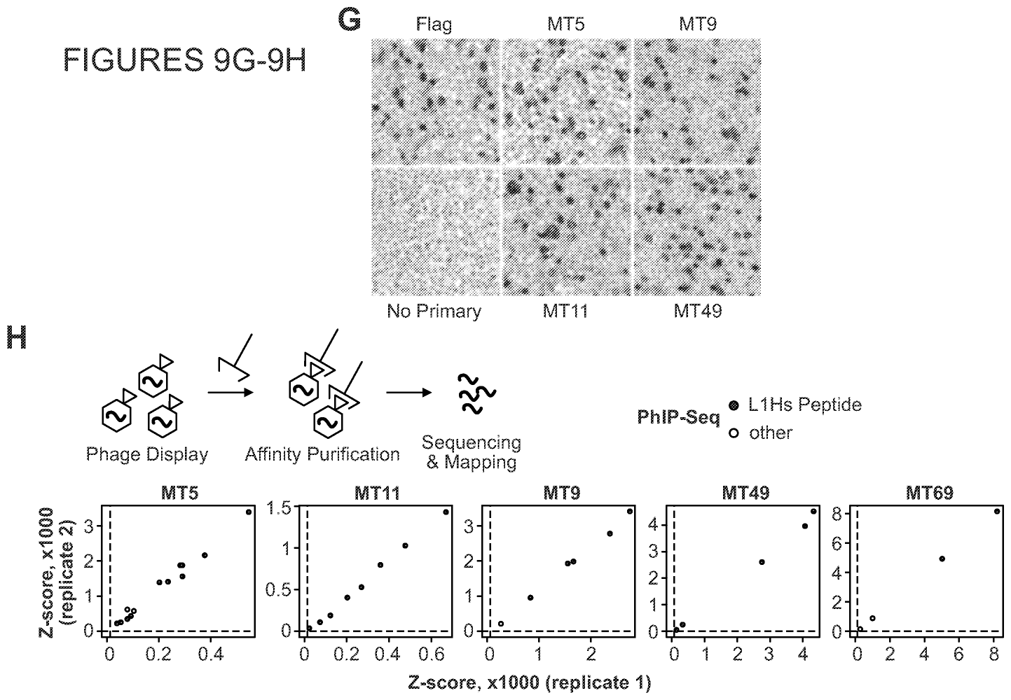

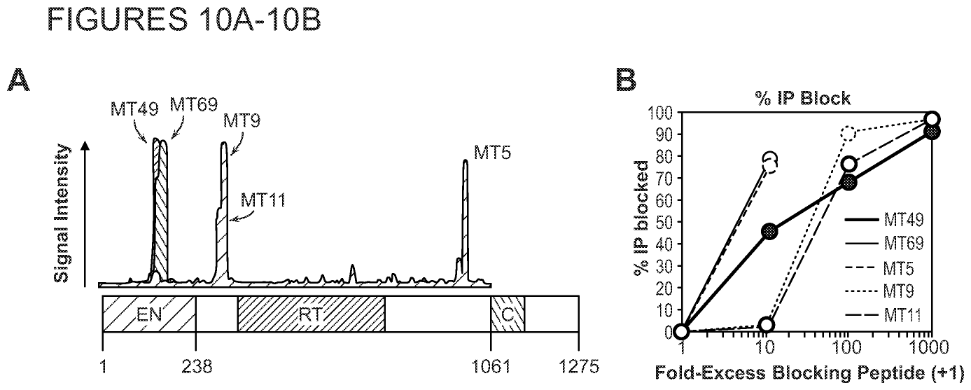

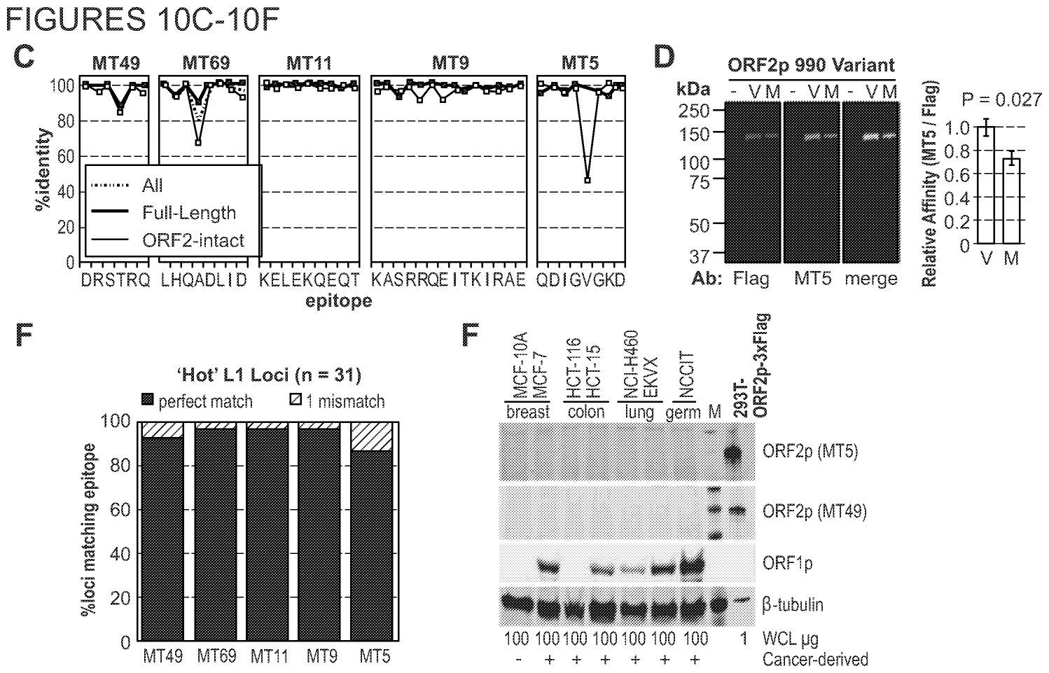

For western blots displayed in FIGS. 9 and 10, cells were lysed in RIPA buffer, vortexed, and supernatants quantified by BCA. Lysates were reduced in LDS with beta-mercaptoethanol and then polyacrylamide gel electrophoresis was performed on 4-20% Protean Mini TGX gels (Biorad) and transferred to Immobilon PVDF membranes for 15 minutes using mini TGX settings on the Trans-Blot-Turbo system (Biorad). Membranes were incubated with primary antibodies overnight at 4° C. (rabbit anti-ORF2 mAbs at 1:1000; mouse anti-Flag M2 (Sigma F1804) at 1:2000), secondary antibodies (all from Licor and used at 1:10,000 dilutions; as appropriate: goat anti-mouse IR680, goat anti-rabbit IR680, goat anti-mouse IR800, goat anti-rabbit IR800) for 1 hour at room temperature, and detection was carried out on the Odyssey Scanner (Licor).

Mass Spectrometry

Peptides were resuspended in 10 μL 5% (v/v) methanol, 0.2% (v/v) formic acid and half was loaded onto an EASY-Spray column (Thermo Fisher Scientific, ES800, 15 cm×75 μm ID, PepMap C18, 3 μm) via an EASY-nLC 1200 interfaced with a Q Exactive Plus mass spectrometer (Thermo Fisher Scientific). Column temperature was set to 35° C. Using a flow rate of 300 nl/min, peptides were eluted in a gradient of increasing acetonitrile, where Solvent A was 0.1% (v/v) formic acid in water and Solvent B was 0.1% (v/v) formic acid in 95% (v/v) acetonitrile. Peptides were ionized by electrospray at 1.8-2.1 kV as they eluted. The elution gradient length was 10 minutes for gel bands and 140 min for all gel plugs except, the second set derived from tumor A, where the gradient length was 190 min. Full scans were acquired in profile mode at 70,000 resolution (at 200 m/z). The top 5 (for gel bands) or 25 (for gel plugs) most intense ions in each full scan were fragmented by HCD. Peptides with charge state 1 or unassigned were excluded. Previously sequenced precursors were also excluded, for 4 s (for gel bands) or 30 s (for gel plugs), within a mass tolerance of 10 ppm. Fragmentation spectra were acquired in centroid mode at 17,500 resolution. The AGC target was 2×10⁵, with a maximum injection time of 200 msec. The normalized collision energy was 24%, and the isolation window was 2 m/z units.

Analysis of Excised Protein Bands and Candidate Phospho-Sites

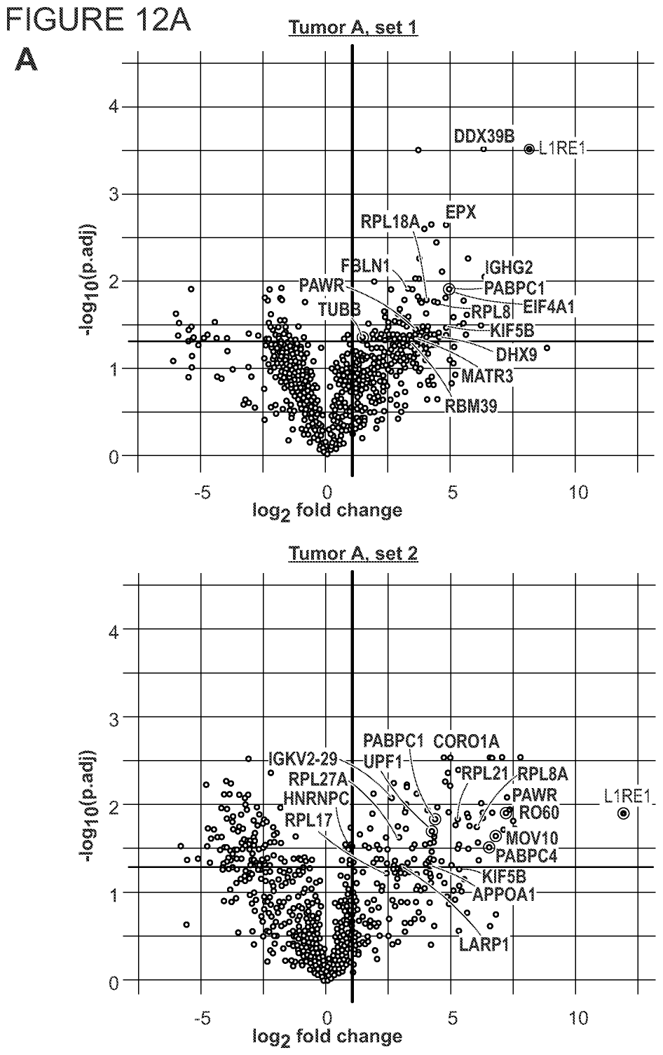

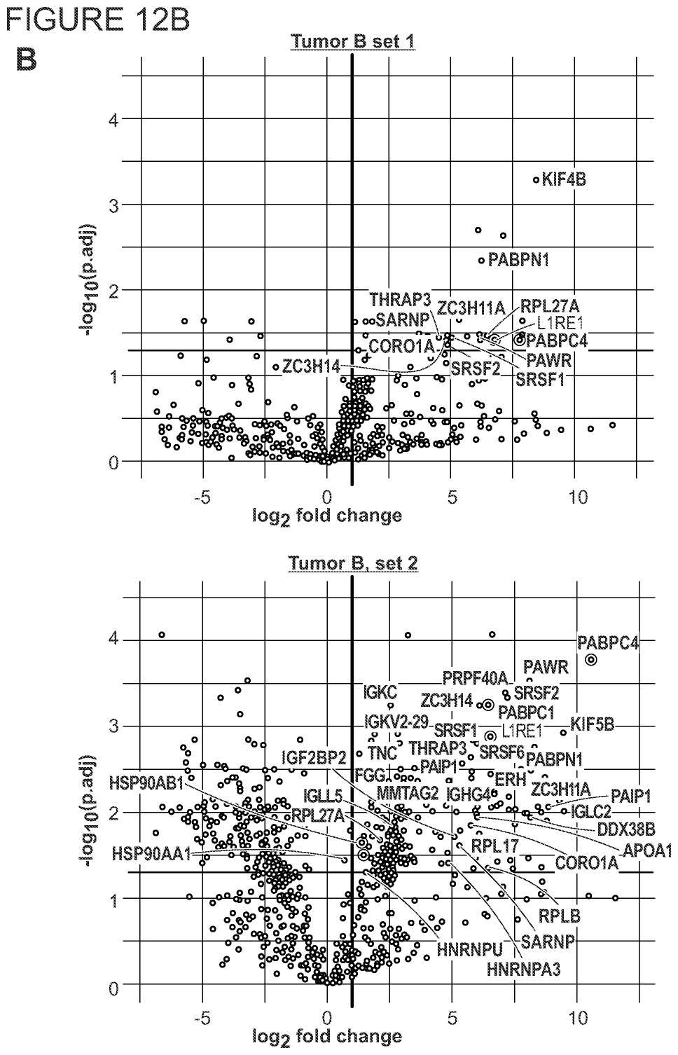

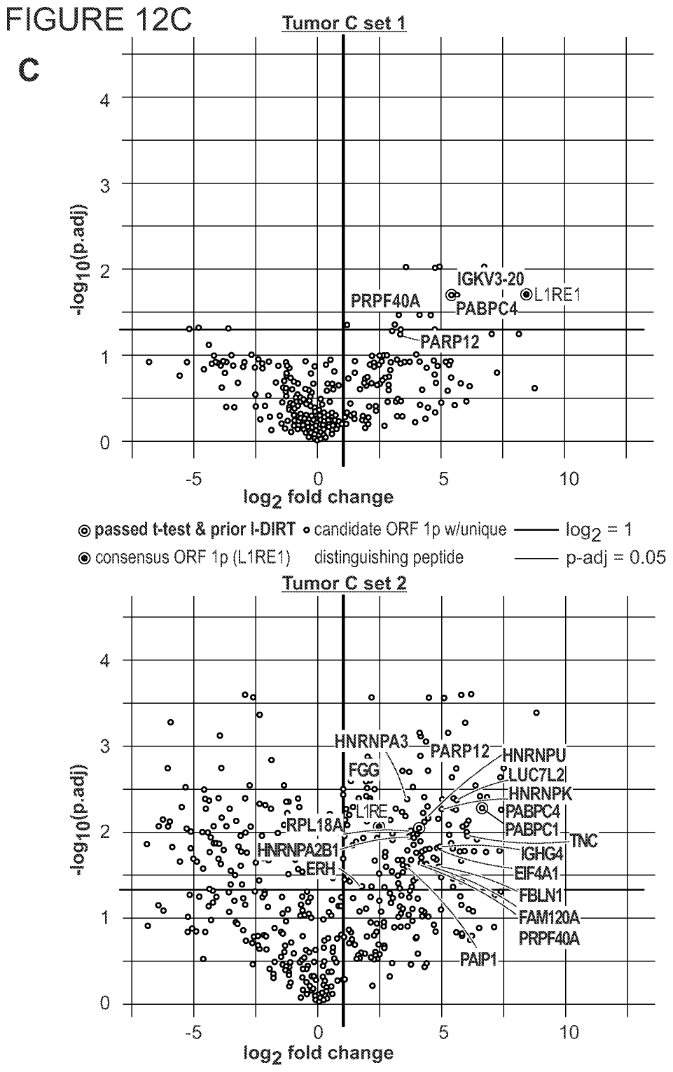

Proteins labeled in FIG. 11A selected for labeling via the following process: The RAW files were converted to MGF format by ProteoWizard and searched against the human protein database with X! Tandem, using the following settings: fragment mass error—10 ppm; parent mass error—10 ppm; cleavage site—R or K, except when followed by P; maximum missed cleavage sites—1; maximum valid peptide expectation value—0.1; fixed modification—carbamidomethylation at C; potential modification—oxidation at M; include reversed sequences—yes. Parameters for the refinement search were: maximum valid expectation value—0.01; potential modifications—deamidation at N or Q, oxidation or dioxidation at M or W; unanticipated cleavage—yes. For each protein ID list, the proteins were ranked by log E-value; keratins, proteins ranked below trypsin, and non-human proteins were removed; if multiple proteins remained, the nth protein (n>1) was removed if (a) it is homologous to a higher-ranked protein or (b) does not have within 50% the number of PSMs of the top-ranked remaining protein; remaining proteins were listed as IDs for each band. For identification of candidate phosphorylation sites using X! Tandem, the RAW files from tumor IPs (corresponding to FIG. 12) were converted to MGF, these were searched against orthogonalized ORF protein sequences (described below), and included the following additional potential modifications during the refinement search: Phospho@S, Phospho@T, Phospho@Y.

Label-Free Quantitative Analysis

Processing RAW data in MaxQuant: we used MaxQuant v1.6.5.0 with default settings and the following adjustments (in brief). Trypsin/P cleavage. Modifications included in protein quantification: Oxidation (M); Acetyl (Protein N-term); Carbamidomethyl (C). Phospho (STY) was searched but excluded from quantification along with unmodified counterpart peptides. Label min. ratio count: 2. Match between runs (within groups of cognate experiments and controls): True. Second peptides: True. Stabilize large LFQ ratios: True. Separate LFQ in parameter groups: False. Require MS/MS for LFQ comparisons: True. We used a protein database composed of the Uniprot human proteome (reviewed), supplemented with non-redundant ORF1p and ORF2p sequences. To increase detection sensitivity, we orthogonalized our ORF1 loci database (from above, Detection of L1 ORF peptides in CPTAC data) within the context of our detected peptides in two steps: (a) retaining loci for which at least one unique peptide was observed and (b) in cases that a peptide was not assigned to any loci in previous step and was commonly shared by several loci, we included only one representative sequence from the group, which was the most different one to consensus ORF1 (L1RE1).

Data preparation. (a) Remove contaminants and reverse protein entries (provided by MaxQuant) and IGHG1. (b) Loge transformation of LFQ MS intensities. (c) Remove proteins with zero values across all cases and controls in a tissue. (d) Impute small values in scenarios that all replicates had zero values for intensity in either cases or controls: we calculated the average (mean) and standard deviation (std) of non-zero values of each replicate and produced small values with the uniform random function between mean—2*std and mean—3*std. (e) Impute values for proteins that have zero intensities in one or two replicates in either cases or controls: we built a distribution of deltas from replicates with non-zero protein intensities:

$$delta = \frac{( {Int}_{rep 1} - {Int}_{rep 2} )}{mean ( {Int}_{rep 1} , {Int}_{rep 2} )} ;$$

Calculate μ_{delta}, Sd_{delta}; Calculate new delta and new Intensity:

$${delta}_{new} = rnorm ( mu = {μ}_{delta} , sd = \frac{{sd}_{delta}}{mean ( correlations ) * \sqrt{2}} ) \\ {I}_{new} = mean ( {Int}_{other} ) * abs ( 1 + {delta}_{new} )$$

Variance Analysis. (a) For tumor A, we performed t-tests between anti-ORF1p IPs and IgG-controls. For tumor B and C, we performed t-tests between tumor and normal tissue after anti-ORF1p IP, as well as between tumor anti-ORF1p IPs and IgG-controls. (b) Adjusted p-values were calculated using Benjamin-Hochberg method. (c) For each entry loge fold change was calculated between case and control average intensity. (d) Significant proteins from all comparison were integrated (p. adjusted ≤0.05 & log2 fold change ≥1). We only accept proteins with (non-imputed) MS intensity values in at least two experimental replicates as candidate true positives. Therefore, proteins that passed ANOVA but were represented by less than two MS-derived intensity values were not considered significant.

Ethics Approval and Consent to Participate

Formalin fixed paraffin embedded (FFPE) and fresh frozen CRC samples were collected at Massachusetts General Hospital Department of Pathology as de-identified patient samples in accordance with Exemption 4 of research involving human subjects from the NIH.

### Example 1

Heterogeneous LINE-1 Expression in Colon Cancer

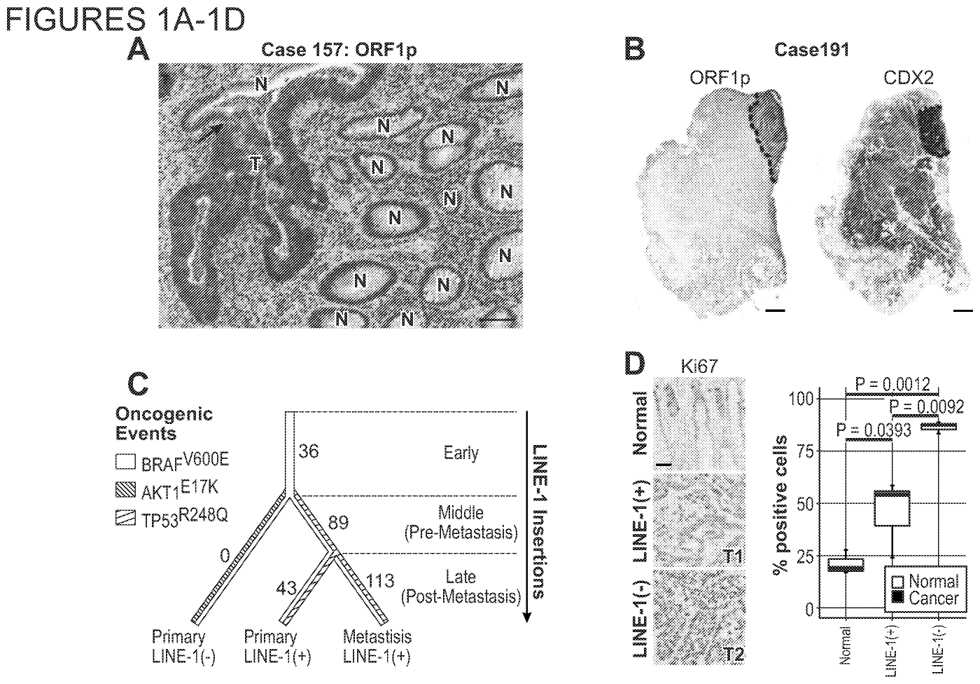

We assessed 22 colorectal cancers (CRC) for ORF1p expression by immunohistochemistry. All were positive, with varied ORF1p staining intensity; immunoreactivity was limited to cancerous epithelium and not found in adjacent normal (FIG. 1A). One tumor showed dichotomous ORF1p expression, containing a well-differentiated LINE-1(+) sector and an adjacent, poorly differentiated (CDX2-dim), LINE-1(−) sector (FIG. 1B). A metastatic site of disease closely resembled the former. To evaluate whether these two tumor regions were clonally related or independently derived, we genotyped driver point mutations and somatically-acquired LINE-1 insertions to create a phylogenetic map (FIG. 1C). We found that the LINE-1(+) and LINE-1(−) parts of the primary tumor both share a BRAFv600E mutation as well as numerous somatically-acquired LINE-1 insertions incurred before retrotransposition ceased in the LINE-1(−) component (data not shown). The LINE-1(−) clone has a markedly increased proliferation index (FIG. 1D). Thus, the LINE-1(−) section derives from a LINE-1(+) lineage, and loss of LINE-1 expression is associated with an enhanced growth rate.

### Example 2

The p53-p21 Pathway Restricts Growth of LINE-1(+) Cells

To identify growth determinants of LINE-1(+) cells, we developed an ectopic expression system in telomerase-immortalized retinal pigment epithelium-1 (RPE) cells, genetically-stable diploid cells with intact p53 and DNA damage responses (FIGS. 2A-B). LINE-1 expression markedly inhibited RPE clonogenic growth 98.2% compared to eGFP control (FIG. 2C). TP53 loss-of-function mutations clinically correlate with LINE-1 activity, so we compared clonogenic growth of RPE cells expressing LINE-1 or eGFP (LINE-1/100 eGFP colonies) with and without TP53 knockdown (FIG. 2D). TP53 knockdown rescued LINE-1(+) cells 42.3-fold but did fully restore to LINE-1(+) cells the clonogenic potential of controls. To test whether TP53 function affects retrotransposition efficiency in this system, we used a reporter assay to compare LINE-1 insertion frequencies in control and TP53 knockdown cells but found no significant difference (data not shown). Thus, TP53 restricts growth of these cells but not retrotransposition potential.

We next performed a genome-wide CRISPR knockout screen to identify knockouts that rescue growth of LINE-1(+) cells (FIG. 2E and Methods). Single-guide RNAs (sgRNAs) targeting TP53 were the only ones to significantly enhance cell fitness (FIG. 2F). Guides targeting CDKN1A (p21), a TP53-mediated growth arrest effector and retrotransposition suppressor, were enriched but did not reach genome-wide significance (FIG. 2F). Guide RNAs targeting other genes downstream of TP53 did not tolerize cells to LINE-1 expression. To validate these findings, we transduced two individual sgRNAs targeting TP53, CDKN1A, or non-targeting controls (NTC) in RPE cells expressing Cas9, and found that each knockout rescued growth of LINE-1(+) cells (FIG. 2G). These data demonstrate that LINE-1 expression causes a p53-p21-dependent growth arrest.

### Example 3

LINE-1 Induces p53-Mediated G1 Arrest and an Interferon Response

To characterize this further, we performed RNAseq in RPE cells encoding a doxycycline-inducible (Tet-On) codon-optimized LINE-1 (ORFeus) or luciferase control (see Methods). In total, 2,261 genes were differentially expressed by more than 2-fold and met Bonferroni-corrected significance (FIG. 3A). Gene set enrichment analysis revealed upregulation of the p53 pathway, and downregulation of cell cycle progression genes (FIG. 3A). Genes possessing p53 regulatory elements (so-called “direct targets”)³³ including CDKN1A (p21) were upregulated in LINE-1(+) cells (p<2.2×10⁻¹⁶), and genes repressed via p21³⁴, were downregulated (p<2.2×10⁻¹⁶) (FIG. 3B). We confirmed by flow cytometry that LINE-1(+) cells accumulated in G1 in a LINE- and TP53-dependent manner (data not shown). LINE-1 expression increases apoptotic effector RNAs PMAIP1 (NOXA) and BBC3 (PUMA), but not caspase 3 activation by western blot (data not shown); Genes associated with the senescence associated secretory phenotype (SASP) were not significantly upregulated (data not shown). These findings are consistent with LINE-1 inducing a p53-mediated G1 cell cycle arrest.

Most (63.6%) of the gene sets upregulated by LINE-1 expression reflect interferon (IFN) signaling (FIG. 3C) and IFN stimulated genes. This appears driven by IFN beta 1 (IFNB1) and the dsRNA sensing pathway TLR3, DDX58 (RIG-I), and IFIH1 (MDA5) (FIG. 3D-E). cGAS-STING is not expressed in these cells. LINE-1 also induces nuclear factor kappa-B (NF-kB)—an immune signaling transcription factor that can be activated by the RNA-sensing pathway—and NF-kB transcriptional targets, including the pro-inflammatory cytokines interleukin-1 beta (IL-1B) and CXCL8 (data not shown). LINE-1 expression in TP53-knockdown cells similarly induces expression of IFNB1 and interferon-inducible genes including TLR3, IFIT 1 and IFIT2, as assessed by qRT-PCR (data not shown), indicating the response is p53-independent. In contrast, addition of nucleoside reverse transcriptase inhibitors known to act on LINE-1, zalcitabine (ddC) or didanosine (ddI), attenuated the IFN response. Thus, LINE-1 expression induces an IFN response which may contribute to its inhibitory effects on cell growth independent of p53.

### Example 4

Mapping LINE-1 Fitness Interactions in TP53-Deficient Cells

We next hypothesized that p53-deficient, LINE-1(+) cells may rely on specific pathways to suppress LINE-1 toxicity. Their loss would be synthetic lethal with LINE-1 expression, and they would be potential therapeutic targets for LINE-1(+) cancers. To identify these pathways, we conducted a knockout screen in TP53-deficient (TP53KD) RPE-Cas9 cells with Tet-On transgenes encoding codon-optimized LINE-1 or luciferase (FIG. 4A and Methods). We generated knockout cell pools in triplicate and expressed LINE-1 or luciferase for 27 days, sampling the populations for sgRNA representation every 4-5 days. Knockouts that become more highly represented in LINE-1(+) cells relative to luciferase(+) controls indicate a positive growth interaction, whereas those that are lost indicate a synthetic lethal interaction. Non-targeting-control (NTC) sgRNAs were equally represented in LINE-1(+) and luciferase(+) cells data not shown). TP53 and CDKN1A knockouts exhibited no difference between LINE-1(+) cells and luciferase(+) cells, confirming that TP53 knockdown effectively inhibited its function and that any p21 growth effects are p53-dependent. As expected, sgRNAs targeting essential genes were depleted from both LINE-1(+) and uciferase(+) populations (data not shown).

We found 1,390 gene knockouts with significant fitness interactions (FIG. 4B see Methods). Only 24 rescued LINE-1(+) cell growth. Knockout of the APC tumor suppressor is among these (data not shown), which is notable since TP53 and APC mutations frequently co-occur in colorectal cancer, and LINE-1 insertions have been shown to disrupt APC in colon cancers. IFNAR1 (IFN receptor) knockout also enhanced cell growth, highlighting that LINE-1-associated IFN activation suppresses cell growth independently of p53. In contrast, most genes identified in this screen (n=1,366) demonstrate synthetic lethal interactions in LINE-1(+) cells within 3 weeks of sustained expression (FIG. 4C).

We asked whether genes known to alter LINE-1 retrotransposition efficiency⁵ or that encode proteins that physically interact with ORF1p or ORF2p were enriched for fitness interactions (FIG. 4D). Of these 239 genes, 59 (24.7%) were identified in our fitness screen, compared to 12.0% (1,390/11,564) of all genes tested, a 2.05-fold enrichment (X²=8.4×10⁻⁹). The majority, 58 of 59 (98.3%), demonstrated synthetic lethal interactions. Of the 59 genes, 10 enhance retrotransposition, 26 suppress retrotransposition, and 25 encode physical interactors. However, these 59 genes only account for 4.2% of genes identified in our study, indicating that most fitness interactors are distinct from host genes that regulate retrotransposition. We conclude that specific gene knockouts cause synthetic lethality in LINE-1(+) cells. Relatively few knockouts act independently of p53 to enhance growth of LINE-1(+) cells, and only a minor proportion of fitness interactors are known to influence retrotransposition.

We performed an overrepresentation analysis on all significant fitness interactors and found a 1.4-fold enrichment of genes encoding nuclear proteins (X²=6.61×10⁻²¹; 50.1% of significant genes compared to 35.2% of genes in the library, see Methods). We found 41 gene ontology (GO) terms with a false-discovery rate (FDR) <0.05 (data not shown). The top enriched term was mRNA processing (FDR=2.29×10⁻¹⁰); we also found terms related to maintenance of genome integrity, including DNA repair (FDR=4.47×10⁻⁷) and DNA replication (FDR=0.01), and chromatin-related gene sets, including histone modification (FDR=3.07×10⁻⁸) and regulation of chromatin organization (FDR=0.001).

### Example 5

HUSH Complex Loss Increases LINE-1 Transgene Expression

Human silencing hub (HUSH) knockouts produced pronounced LINE-1 synthetic lethal interactions which we validated by single gene knockout clonogenic growth studies (data not shown). HUSH is an epigenetic repressor complex that targets transgenic DNA sequences including lentivirus insertions and endogenous LINE-1 loci. Thus, we tested whether HUSH loss increases LINE-1 expression, either from endogenous LINE-1 loci or from the codon-optimized transgene. We did not detect ORF1p or ORF2p in no-doxycycline controls (data not shown), indicating that HUSH mutant RPE cells do not upregulate endogenous LINE-1 proteins. In doxycycline-treated cells with the LINE-1 transgene, ORF1p, ORF2p, and transgene mRNA expression increased with HUSH knockout and ORF2p protein level linearly correlated with transgene mRNA level (2-4 fold increase, data not shown). ORF2p expression could be similarly increased in cells with intact HUSH by higher doses of doxycycline, and this is highly cytotoxic. We conclude that the synthetic lethal effect of HUSH mutants is caused by enhanced expression of the LINE-1 transgene. We note that high levels of ORF2p expression overwhelm the survival advantage conferred by TP53 deficiency.

### Example 6

RNA Processing Gene Knockouts Sensitize Cells to LINE-1 Expression

The GO term mRNA processing encompasses 81 genes demonstrating fitness interactions in LINE-1(+) cells; these genes are enriched for spliceosome components (P=2.24×10⁻³⁴) and knockouts of these are synthetic lethal in LINE-1(+) cells (data not shown). We validated this effect by treating cells with the splicing inhibitor pladienolide B (PLA-B), which acts on the essential gene SF3B1 (splicing factor 3b subunit 1), a component of the U2 snRNP. At a PLA-B dose that reduced luciferase(+) clonogenic growth by 6.8%, LINE-1(+) cells grew 27.8% fewer colonies, a 4.1-fold increased sensitivity to PLA-B (P=0.044). We analyzed RNAseq data from LINE-1(+) RPE and did not observe alternatively spliced isoforms of the LINE-1 transgene (data not shown), indicating that these gene knockouts likely impact cell growth through an indirect mechanism rather than by directly processing the LINE-1 RNA. Notably, cells subjected to ultraviolet or ionizing radiation DNA damage also are sensitized to loss of spliceosome components.

We found pronounced synthetic lethal interactions caused by knockouts of genes encoding the nuclear exosome targeting (NEXT) complex, which degrades intronic RNAs and processed transcripts. Two of the three complex members demonstrate synthetic lethal interactions (RBM7 and ZCCHC8) whereas the third (SKIV2L2) is an essential gene (data not shown). Similarly, RNASEH2 knockout is synthetic lethal in LINE-1(+) cells. RNASEH2 facilitates retrotransposition by degrading LINE-1 RNA from RNA-DNA hybrids after reverse transcription occurs. Thus, when RNASEH2 is lost, this precludes LINE-1 retrotransposition and enhances toxicity.

Finally, we find that LINE-1(+) cells require the dsRNA adenosine (A) to inosine (I) editing enzyme ADAR1 (data not shown), as do cancer cell lines with high expression of interferon stimulated genes.

### Example 7

Fanconi Anemia Proteins Suppress LINE-1 Toxicity

DNA repair genes that suppress LINE-1 toxicity were enriched for Fanconi Anemia (FA)-BRCA1 pathway components (P=7.65×10⁻¹³, FIG. 5A). The FA pathway is critical for resolving DNA interstrand crosslinks and transcriptional R-loops that interfere with progression of DNA replication. Knockout of the majority (83%) of the genes known to cause FA and several related genes exhibited synthetic lethal interactions with LINE-1 (FIG. 5B), including BRCA1 (FANCS). We chose five genes to validate based on their functions in the pathway: FANCM, a highly-conserved helicase and branch translocase that has high affinity for stalled replication forks and RNA:DNA hybrids; FANCA, which is required for FA “core complex” assembly; FANCL, the E3 ubiquitin ligase that activates the downstream effectors of the “ID Complex,” FANCI and FANCD2. We confirmed knockout efficacy by measuring mitomycin C (MMC)-induced FANCD2 monoubiquitination (FANCD2-Ub) (FIG. 5C). MMC induced FANCD2-Ub in NTC-treated cells but not in the FA knockouts. These FA-deficient mutants were selectively sensitive to LINE-1 expression compared to NTCs (FIG. 5D) and displayed slight increases in chromatin-bound γH2A.X compared to NTC-treated LINE-1(+) cells (1.1-1.7 fold, data not shown). Expression of native LINE-1 sequence is also synthetic lethal in FANCD2-knockout cells compared to NTC controls (data not shown).

Based on these data and reports that FA proteins suppress retrotransposition, we hypothesized that the FA pathway is activated by LINE-1. To test this, we measured monoubiquitination of FA effector proteins FANCD2 and FANCI and found 1.6- and 1.5-fold increases, respectively, with LINE-1 expression (FIG. 5E). Importantly, LINE-1 cytotoxicity has been previously reported to depend on endonuclease (EN) and reverse transcriptase (RT) activities, and we confirmed that expression of LINE-1 with inactivating EN and RT mutations is less toxic than wildtype (WT) LINE-1. To dissect whether the enzymatic activities of LINE-1 are necessary for FA activation, we measured FANCD2 monoubquitination in HeLa cells expressing WT LINE-1 or mutants lacking EN activity and/or RT activity. Whereas WT LINE-1 increased FANCD2-Ub (2.6-fold), both EN-(H230A) and RT-(D702Y) inactivating mutations did not (FIG. 5f). We next assessed FA activation by enumerating FANCD2 nuclear foci. We expressed WT or RT mutant LINE-1 and quantified FANCD2 nuclear foci in randomly-imaged, EdU-labeled cells (see Methods). Both hydroxyurea (HU) treatment and LINE-1 expression increased the number of FANCD2 foci in S phase (EdU+) cells (p=1.7×10⁻⁸ and 5.8×10⁻¹¹, respectively, FIG. 5G) but not in G1/G2 (EdU−) phase. The LINE-1 RT mutant did not induce FANCD2 foci formation. Together, these data demonstrate that LINE-1 activates the FA complex and replication-coupled DNA repair. By contrast, LINE-1 EN and RT mutants do not have this effect, suggesting that the LINE-1 retrotransposition intermediate is crucial to the process.

To evaluate DNA damage associated with LINE-1 expression, we measured γH2A.X and 53BP1 nuclear foci. We found that LINE-1(+) cells have transient increases in numbers of γH2A.X and 53BP1 foci as compared to control cells (p=3.4×10⁻⁶ and 1.7×10⁻, respectively, FIG. 5H). These increases are detectable in S phase and resolve by G2 whereas doxorubicin-induced DNA damage foci continue to accumulate (data not shown). This pattern is more consistent with LINE-1-induced replication stress than with a large burden of persistent, dsDNA breaks.

### Example 8

Retrotransposition-Replication Conflict Underpins LINE-1 Toxicity

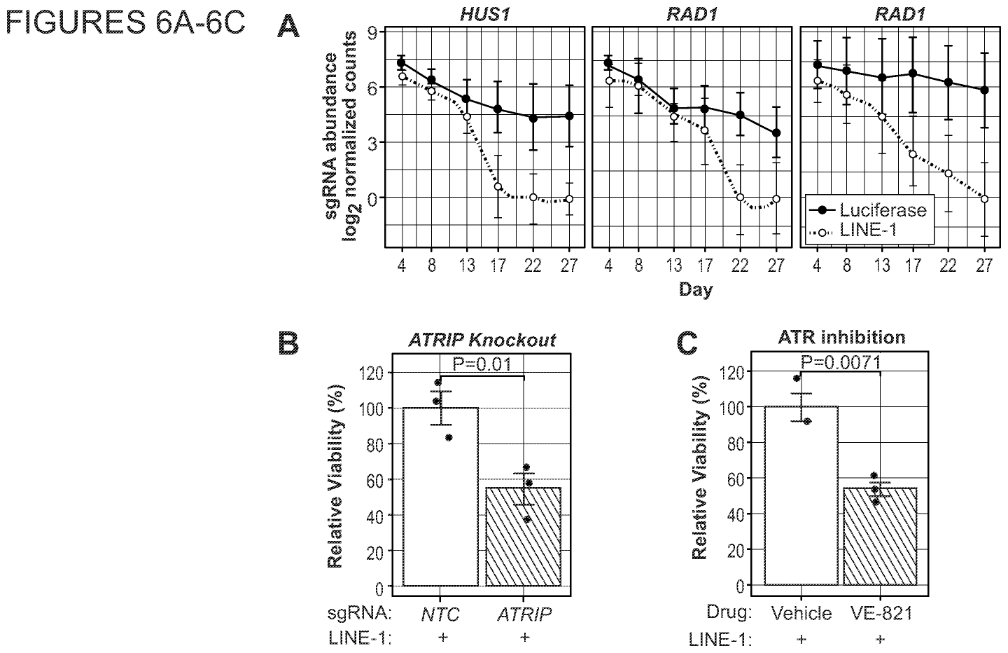

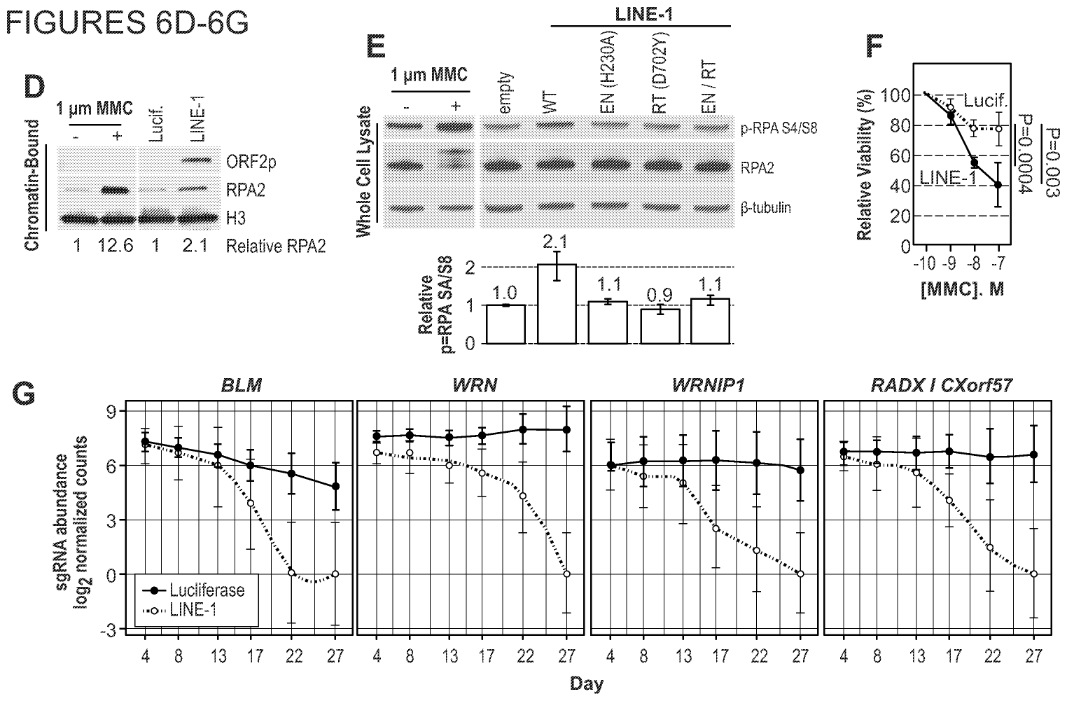

We next explored interactions between LINE-1 retrotransposition and DNA replication using our fitness screen data. Stalled replication forks activate signaling pathways involving ATR (Ataxia Telangiectasia and Rad3-Related) and ATRIP (ATR-interacting protein), as well as the tripartite RAD9, HUS1, RAD1 (9-1-1) complex. ATR and RAD9 are essential, but genes encoding all non-essential components of these complexes (ATRIP, HUS1, and RAD1) are synthetic lethal LINE-1 interactors (FIG. 6A). We validated that ATRIP knockout cells exhibited heightened sensitivity to LINE-1 expression (FIG. 6B); they also failed to sufficiently activate FANCD2 upon MMC-induced DNA damage (data not shown). Similarly, ATR inhibition with VE-821 sensitized cells to LINE-1 (FIG. 6C) at a dose that had no effect on viability in luciferase(+) cells (data not shown). Thus, compromising replication stress signaling is synthetic lethal in LINE-1(+) cells, potentially related to the role of ATR-ATRIP signaling in activating the FA pathway.

We next assayed for signs of replication fork stall. Stalled replication forks accumulate ssDNA coated by RPA, a heterotrimer comprised of RPA1, RPA2 and RPA3, to protect genomic DNA from nucleases. We isolated chromatin-bound protein fractions from cells treated with MMC or expressing LINE-1 or luciferase and found that both MMC treatment and LINE-1 expression induced chromatin-bound RPA2 (FIG. 6D). These data show replication stress occurring in a LINE-dependent manner. We next asked whether LINE-1-associated replication stress depends on ORF2p enzymatic activity. We expressed WT or mutant LINE-1 from Tet-On plasmids in HeLa cells and measured p-RPA S4/S8, a phosphorylation modification placed on RPA during replication stress. WT LINE-1 significantly induced p-RPA S4/S8 by 2.1-fold (p=0.0007), whereas EN- and RT-inactivating mutations did not (FIG. 6E). These data indicate that ORF2p must nick DNA and reverse transcribe in order to induce replication stress, highlighting the importance of the retrotransposition intermediate in these events. Moreover, LINE-1(+) cells were 1.9-fold more sensitive to mitomycin C (MMC) as compared to luciferase-expressing controls (FIG. 6F). Together, these data indicate that LINE-1 retrotransposition induces replication stress and sensitizes cells to compounds that increase demands on replication-coupled DNA repair.

Several key processes occur downstream of replication stress signaling, including: (i.) fork reversal, (i.e., translocation of the replication fork away from the lesion and resection by nucleases including ZRANB3, SMARCAL1, and HLTF), (ii.) fork protection from excess degradation by nucleases, and (iii.) fork restart. Fork reversal genes do not score in our screen, whereas the fork protection factor RADX and proteins that are important for fork restart—including Bloom helicase (BLM), Werner helicase (WRN) and WRN interacting protein 1 (WRNIP1)—are LINE-1 synthetic lethal interactors (FIG. 6G). Fork restart additionally requires the removal of RPA from the ssDNA. To this end, we note that knockout of RFWD3, an FA member whose E3 ubiquitin ligase activity regulates RPA unloading from chromatin, produces synthetic lethality (data not shown). These findings indicate that replication fork protection and restart, but not reversal, are essential for LINE-1 cell growth.

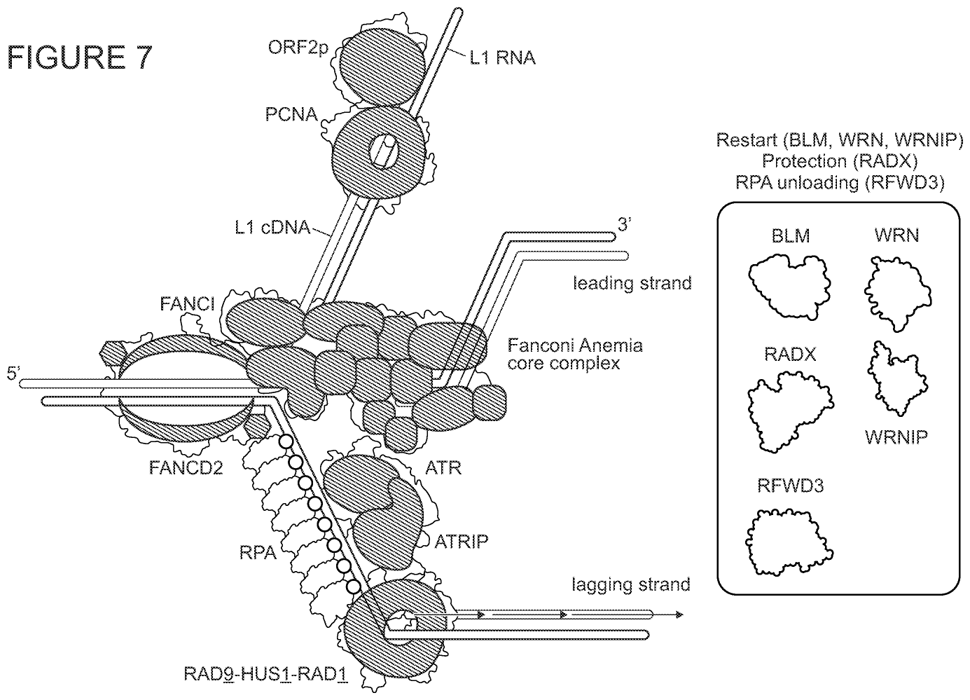

Taken together, these data are consistent with a model wherein LINE-1 retrotransposition intermediates cause replication stress (FIG. 7). LINE-1(+) cells rely on FA-mediated DNA repair, replication stress signaling, and fork restart pathways for growth.

Our findings underscore that limits on LINE-1 expression are required in order to preserve cell growth, and indeed we began our study based on evidence of one tumor that lost LINE-1 expression and subsequently grew faster. Moreover, we provide the first evidence of unique molecular vulnerabilities in LINE-1(+) cells, which has significant implications for translational cancer research. From a therapeutic perspective, it is possible that LINE-1(+) cancers will have characteristic drug sensitivities; for example, LINE-1 ORF2p expression and retrotransposition may prove a biomarker for tumors that respond to DNA damaging agents, or inhibitors of ATR or WRN helicase. We also demonstrate that LINE-1 promotes a type I interferon (IFN) response, suggesting roles for LINE-1 in sensitivities to immunotherapies or ADAR inhibition.

### Example 9

Detecting ORF1p and ORF2p Peptides in Tumor Mass Spectrometry Data

We reanalyzed data from Clinical Proteomics Tumor Analysis Consortium (CPTAC) to assess L1 ORF1p and ORF2p protein production in tumors. CPTAC has generated deep mass spectrometry based proteomics data from treatment naive breast and ovarian tumors using isobaric labeling and extensive prefractionation with alkaline reversed-phase chromatography followed by inline (acidic) reversed-phase chromatography and Orbitrap mass spectrometry. For the detection of ORF1p and ORF2p peptides, we constructed a protein sequence collection that, in addition to human proteins from Ensembl, also included high confidence LINE-1 protein coding sequences from L1Base2, and used the X! Tandem search engine with the curated databases and the same search parameters as Ruggles et al.

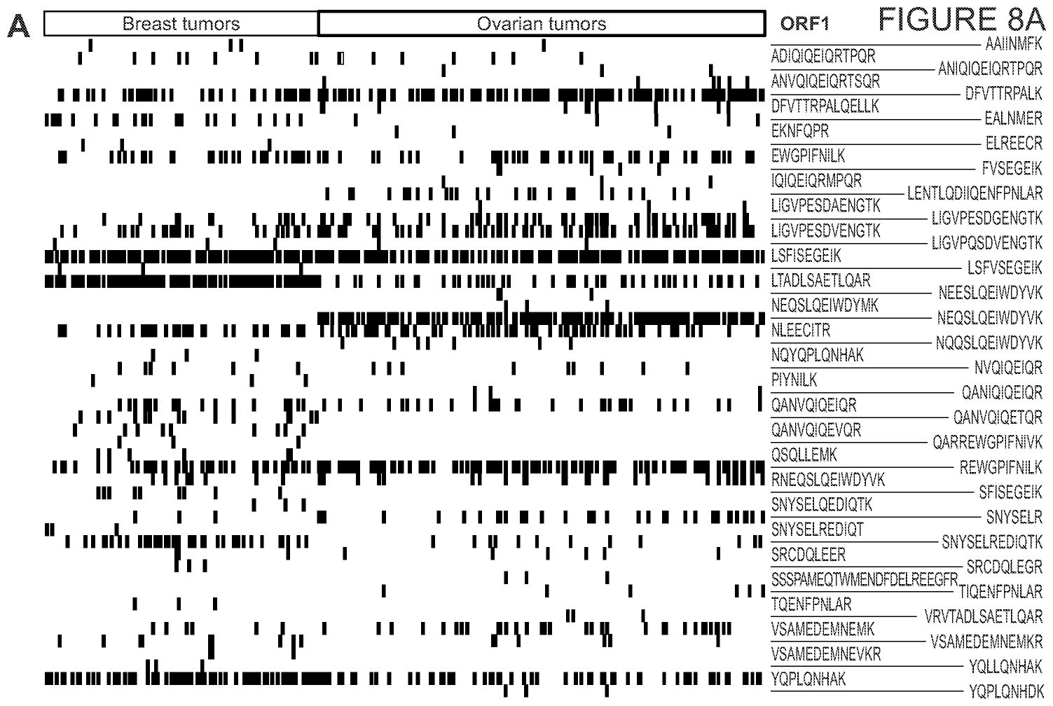

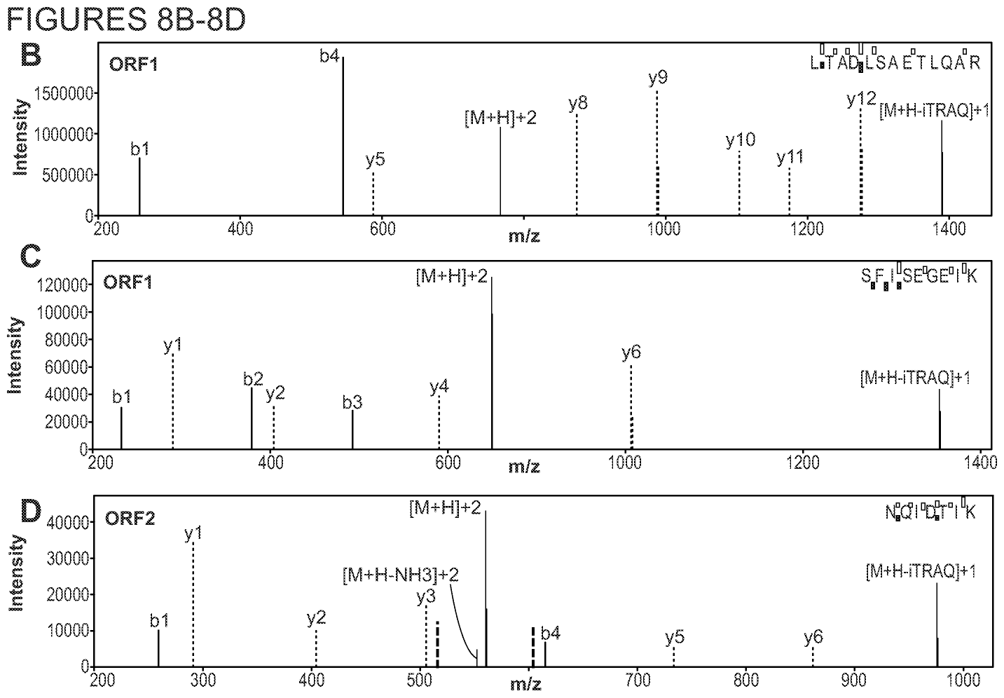

We observed ORF1p in most breast and ovarian tumors (FIG. 8A, with several peptides observed for the majority of tumors; see FIG. 8B and 8C for two examples of quality ORF1p peptide spectrum matches [PSMs]), but there was no clear evidence for ORF2p. Even when we relaxed the filters, the potential evidence for ORF2p peptides was questionable and the majority of PSMs were semi-tryptic and had borderline e-values 0.01). We also inspected the potential ORF2p PSMs manually and rejected them because they had several large peaks that could not be explained by fragmentation of the assigned ORF2p peptide. The best ORF2p PSM and only potential evidence for ORF2p that was not rejected is shown in FIG. 8D, but this peptide is short and still has two prominent peaks that are not explained by the sequence. In summary, we can reliably observe ORF1p in breast and ovarian tumors using deep mass spectrometry-based proteomics, but, in contrast, the evidence for detection of ORF2p is inconclusive.

### Example 10

Monoclonal Antibodies Detect Human LINE-1 ORF2 Protein

To pursue targeted ORF2p detection methods, we chose the retrotransposition-competent L1RP sequence as an immunogen for generating ORF2p monoclonal antibodies. L1RP is part of the highly active Ta-1d subfamily of L1, which encompasses the vast majority of hot L1s found in humans (including LRE3, L1RP, L1.3). Prior to immunization in rabbits, we expressed tagged ORF2 fragments from bacteria, one fragment with the endonuclease domain (EN, amino acids 1-238, His6 tag) and one fragment containing the reverse transcriptase domain and surrounding sequence (RT, amino acids 238-1061, tagged with mannose binding protein/MBP or a small ubiquitin-like modifier/SUMO) (FIG. 9A). We also expressed full-length Flag-tagged ORF2 (ORF2-3× Flag) in Tet-On human embryonic kidney-293T (HEK-293T_{LD}) cells to screen immune sera. We confirmed fragment purity after Nickel or size-exclusion chromatography (FIG. 9B).

For EN-targeting antibodies, we immunized and boosted two rabbits with EN-His6 fragments, then screened hybridoma supernatants by ELISA against purified EN domain and subsequently ORF2-3× Flag. We used the same strategy for RT-targeting antibodies, but used MBP-RT to stimulate the primary immune response and boosted with SUMO-RT to avoid MBP-specific antibody generation. We counter-screened against MBP and SUMO immunoreactivity to identify hybridomas with reactivity for ORF2 (FIG. 9C). Hybridoma supernatants were then tested for their ability to detect ORF2-3× Flag by western blot (FIG. 9D), IP (9E), immunofluorescence (IF, 9F), and IHC (9G). We used Flag-antibody as a control to determine whether our ORF2 antibodies detected ORF2-3× Flag. Five monoclonal antibodies (mAbs) were selected based on their ability to detect full-length ORF2-3× Flag by each modality: MT5, MT9, MT11, MT49, and MT69.

### Example 11

ORF2 Antibodies Identify Non-Overlapping Epitopes

There are several hundred potentially active L1 loci with intact open reading frames that have been characterized in modern humans, and these sequences are highly identical to one another at both the nucleotide and amino acid level. The specific repertoire of L1 loci that is expressed varies among individuals, and by cell or tissue type. To evaluate the potential of our mAbs to detect proteins originating from distinct copies of L1Hs, we mapped the specific epitopes recognized by each antibody and evaluated the conservation of each epitope among L1Hs loci.

We mapped target epitopes using a peptide array of overlapping 15-mers tiling the length of ORF2p. We incubated these arrays with each antibody and then used secondary antibodies conjugated to horseradish peroxidase (HRP) to identify which peptides were identified. Epitopes were identified as the largest contiguous stretch of amino acids that showed mAb binding over background (FIG. 10A). The linear epitopes ranged in length from 6 to 14 amino acids. Each of the 5 epitopes mapped to a discrete, non-overlapping segment of ORF2 in a manner consistent with the purified protein fragments we used for rabbit immunization. The MT49 epitope (DRSTRQ) (SEQ ID NO: 1) and MT69 epitope (LHQADLID) (SEQ ID NO: 2) occur adjacent to one another and target amino acids on the surface of the endonuclease domain according to a published crystal structure. Both the MT9 epitope (KASRRQEITKIRAE) (SEQ ID NO: 3) and MT11 epitope (KELEKQEQT) (SEQ ID NO: 4) are located between the annotated EN and RT domains, whereas MT5 identifies an epitope (QDIGVGKD) (SEQ ID NO: 5) ˜300 amino acids from the C terminus, adjacent to the C domain.

To validate these epitopes, we pre-incubated mAbs with blocking peptides and attempted IP of ORF2-3× Flag. We found a concentration-dependent blocking activity of each peptide on its corresponding mAb; a range of 10-1000-fold excess peptide was required to achieve this effect depending on the mAb (FIG. 10B). These findings confirm that these epitopes are the antibody targets. PhIP-seq data were also consistent with these being the cognate epitopes recognized by each antibody. Finally, we complemented our finding of antibody specificity by PhIP-seq by performing a BLAST search of these epitopes, which revealed that the only perfect matches in the human genome belong to L1 ORF2p sequences.

### Example 12

ORF2 mAbs are Sensitive for Many Genomic Source Elements

To evaluate the occurrence of these epitopes in naturally-occurring L1 sequences, we used a census of fixed and commonly-occurring potentially protein-coding L1 elements found in the hg38 reference genome build. We focused on those with intact ORF2 reading frames as previously annotated by L1Base. We performed clustal alignments for two non-overlapping sets of these elements, one consisting of 146 full-length loci (111 L1Hs, 35 L1PA2) and one with 107 ORF2-intact loci. We included consensus sequences of the youngest human-specific L1 (L1Hs) and next-youngest primate-specific L1 (L1PA2) as well as the sequence of L1RP—the antigen against which our mAbs were raised—to compare sequences of the immunogen used for antibody generation against those of other genomic L1 loci. Full-length, intact LINEs are predominantly of the species-specific L1Hs subfamily, but include some older, primate-specific L1 elements. As expected, full-length and ORF2-intact L1 amino acid sequences are nearly identical for this set (data not shown). L1RP-encoded ORF2p is 1,275 amino acids long. Individual, full-length L1 elements had open reading frames that differed from this on average by 16 amino acids (1.25%, range 1-61, data not shown), and ORF2-intact L1 loci differed on average by 32 amino acids (2.5%, range 2-79, data not shown).

To assess which of the several hundred reference L1Hs loci could be detected by our mAbs, we used these clustal alignments to evaluate the proportion of L1 loci matching each mAb epitope (FIG. 10C). For each epitope, most full-length L1 loci have amino acid sequences that are 100% identical. The MT11 epitope (KELEKQEQT) (SEQ ID NO: 4) and MT9 epitope (KASRRQEITKIRAE) (SEQ ID NO: 3) similarly occur nearly universally in ORF2-intact L1 sequences. The greatest discrepancy occurred in the MT5 epitope (QDIGVGKD) (SEQ ID NO: 5), where amino acid position 990, which tends to be universal in intact, full-length L1 sequences, is not consistently found in elements selected only for an intact ORF2. Position 990 is typically a valine in L1Hs sequences and a methionine in older elements such as L1PA2 due to a G>A nucleotide substitution (ORF2 position 2,968). We tested whether substituting the L1Hs valine for methionine was sufficient to preclude antibody recognition of the epitope. We created Flag-tagged L1RP ORF2p with an M990 substitution, expressed both protein variants in HEK-293TLD, and performed a western blot using both MT5 and anti-Flag antibodies (FIG. 10D). We detected no signal from MT5 or anti-Flag in untransfected cells. We detected both V990 and M990 variants by both anti-Flag and MT5 antibodies. The M990 variant was detected at a weaker relative intensity by MT5 compared to anti-Flag, indicating a reduced affinity for the ancestral (M990) sequence compared to the derived (V990) sequence. Consequently, we concluded that this single amino acid change within the target epitope may reduce detection sensitivity but would not prevent detection of L1PA2-encoded ORF2p by this reagent. Among 31 loci previously reported to be ‘hot’ or highly active elements, mAb epitopes differ by at most 1 amino acid from the L1RP variant (FIG. 10E), suggesting that these youngest L1Hs sequences are likely identifiable by all of the 5 antibodies. It is important to note that these reference L1 sequences do not capture all of the sequence variation within the many polymorphic L1 alleles that are currently segregating in human populations; these are likely close in sequence to the L1Hs consensus but will differ by some number of amino acids. Thus, without having actual sequence data, our reagent may fail to bind some variants. However, we expect that our mAbs can detect the large majority of active L1 encoded in the human genome. We thus probed ORF2p expression by western blot in a panel of cancer cell lines known to express ORF1p but were unable to detect it (FIG. 10F).

### Example 13

Characterizing L1 Immunoprecipitates from CRCs

We next pursued ORF2p detection after first affinity enriching ORF1p from tumor extracts. In our prior work with ectopically expressed L1 RNPs in HEK-293T_{LD} cells, we readily co-immunoprecipitated ORF1p/ORF2p/L1 RNA-containing macromolecules, and robustly detected ORF proteins. The detection of ORF2p had, at the time, been a widely recognized problem which we addressed by appending a 3× Flag epitope-tag to the protein. The 3× Flag tag allowed us to robustly capture and detect ORF2p. Because these experiments provided a window on L1 biology only in an ectopic expression context, we wanted to evaluate concordance with pathophysiology. To this end, we sought to isolate L1 RNPs, directly from ORF1p-expressing tumors using an anti-ORF1p affinity medium, comparing and contrasting the results obtained with those from our studies of ectopic L1 expression. We obtained a cohort of CRCs that were shown to be ORF1p positive (+) by immunohistochemistry (IHC) and carried out a preliminary proteomic characterization of three tumors selected from across the ORF1p+ IHC staining spectrum. FIG. 11 shows the results we obtained by multiple proteomic methods. Tumor A was the highest ORF1p-expressing case among this group. Tumor C was on the low-to-moderate-end of the expression spectrum and did not yield a distinct, visible ORF1p band after immunoprecipitation (IP): FIG. 11A, compare the ORF1p staining intensity in the first (far left, Tumor A), fourth (Tumor B), and eighth (Tumor C) lanes of the gel; 11B exhibits results obtained with Tumor A using a modified procedure (see FIG. 11 legend and Methods). Also, see FIG. 11C for a comparison of ORF1p yield after IP from our highest-expressing ectopic system (pLD401) to tumors A and B. Although IP from these materials yields ORF1p quantities that are directly comparable, side-by-side western blotting of cell extracts revealed that ectopic expression produced a significantly higher level of ORF1p than these tumors (FIGS. 11D). Surprisingly and importantly, ORF2p is only detected on our western blots in the ectopic expression positive control (FIGS. 11D, 11E). The result did not change when extending the blot exposure time from 2 min (shown) to 30 min (not shown); nor was the result different when using alternative anti-ORF2p antibody clones characterized in this study. Presuming ORF2p has been retrieved from these tumors by co-IP with ORF1p, we conclude the yield is below the lower limit of detection of our blotting, under the conditions tested. FIG. 11F shows cognate anti-ORF1p IHC staining results for tumors B and C-corroborating the signal intensity difference revealed by western blotting.

To contextualize the results obtained with CRCs, we developed a similar analysis in a broader selection of cell lines (FIG. 11E). We observed that the yield of endogenous ORF1p by IP was ≲1/10^{th} the amount observed in HEK-293T_{LD} expressing L1 ectopically from pMT302. This construct was chosen on account of its milder ectopic expression level. Modified from a naturally occurring L1 sequence (L1RP), expression from pMT302 has been estimated to yield ˜1/40^{th} the L1 RNA and ORF2p and ˜1/4^{th} the ORF1p expression typically observed from codon-optimized L1 encoded by pLD401. PA-1, an ovarian teratocarcinoma cell line known to be permissive for the expression of endogenous L1, stood out among this group—demonstrating ˜1/10^{th} the ORF1p yield of pMT302 and the highest yield from an endogenous context in this panel. Western blotting also demonstrated ORF1p signal in cell lysates from the panel, but only under probing conditions that increased high mass (nonspecific) signal in the blot (data not shown). In this panel, ORF2p was not detected, except by co-IP with ORF1p from pMT302.

Believing we exhausted the potential of western blotting for ORF2p detection, we turned to MS-based proteomic analyses. FIG. 12 displays the results of a label-free, quantitative MS analysis of affinity captured ORF1p, from the same tumor samples displayed and analyzed in FIG. 11. As expected, we identified L1RE1 (consensus ORF1p) as a significantly enriched protein in each IP set. Taken all together, we observed eight other proteins that we have previously characterized as putative physiological L1 interactors (PABPC1, PABPC4, TUBB, RO60, UPF1, MOV10, HSP90AA1, HSP90AB1); PABPC1/4 being most frequently recovered. We explored the interactors discussed in, originating from two studies, conducted by the Moran and Kazazian labs. We observed DHX9 and MATR3 (in Tumor A, set 1), HNRNPC and LARP1 (in Tumor A, set 2), SRSF1 (in Tumor B, set 1 & Tumor B, set 2), SRSF6 and IGF2BP2 (Tumor B, set 2), HNRNPU (in Tumor B, set 2 & Tumor C, set 2), and FAM120A and HNRNPA2B1 (in Tumor C, set 2). Only HNRNPU was observed to be a significant hit in two different patient tumors. Notably, HNRNPU, DHX9, MATR3, HNRNPC, and other RNA binding proteins have been reported to accumulate on L1 and retro-element-derived RNAs; in one hypothesis, insulating these sequences from nuclear RNA processing pathways that might otherwise be deleterious to the retro-element and host genes harboring these sequences.

The data can be summarized as follows: 291 proteins were detected as significant in one comparison (tumor vs. control IP: p-adjusted value of ≤0.05 and loge fold change >1), 37 passed two comparisons, and 22 passed three comparisons; 21 ORF1p candidates with one or more mutations from consensus ORF1p were detected and of these 12 were observed in both tumor A and tumor B. We observed a candidate phosphorylation site at S18 (156 PSMs from this study). The next most frequent candidate phosphorylation site, S27, received only 31 PSMs; both S18 and S27 phospho-sites have previously been reported and have been implicated as (1) functionally important for retrotransposition and (2) mediating an interaction with the peptidyl-prolyl cis/trans isomerase PIN1. Importantly, we did not detect ORF2p in any of these tumor analyses.

The above proteomic analyses were conducted under the assumption that these tumors will harbor somatic retrotransposition events based on what has been reported in the literature. Nevertheless, we could not rule out the possibility that these tumors did not express ORF2p. We therefore selected a tumor (tumor D: a sigmoid colon cancer that metastasized to liver) and carried out transposon insertion profiling by sequencing (TIP-seq) to map new insertions and establish ORF2p activity. TIP-seq analysis revealed somatically acquired insertions in the primary tumor and its metastatic sites (data not shown). On this basis we conducted co-IP/western blot analysis on the same material in an effort to detect ORF2p (data not shown). No ORF2p was detected, verifying that even when new insertions are observed to occur, and when ORF1p has first been highly enriched, ORF2p detection remains challenging.

### Example 14

Endogenous ORF2p Expression Cannot Presently be Directly Detected in Human Cancers

In summary, given the lack of ORF2p detection by mass spectrometry of tumor extracts (FIG. 8), we tested for endogenous ORF2p expression in human cancers tissues and cell lines by western blotting, IP-western, IP-MS, and IHC (FIGS. 10-12). Western blotting and IP-western in several widely used human cell lines expressing endogenous ORF1p showed no evidence of ORF2p expression (FIGS. 10F and 11E). We also conducted IHC in human CRCs with these reagents, including cases known to sustain somatic retrotransposition. ORF1p was readily detectable in all cases evaluated. ORF2p immunostaining, by contrast, showed no consistent signal over isotype controls under standard conditions. Under conditions employing a highly sensitive protocol, immunoreactivity over the isotype control was apparent only inconsistently as cytoplasmic staining with one of the antibodies (MT49, data not shown).

We have evaluated L1 ORF2p expression in human cancers using several independent and orthogonal approaches—one reliant on whole proteome analysis; one employing a series of new, apparently avid and specific monoclonal antibodies for ORF2p detection; and one leveraging ORF1p interactions to seek evidence of ORF2p. While many types of epithelial cancers express levels of ORF1p that are directly detectable by western blotting and mass spectrometry, ORF2p in these cases appears to be only indirectly detectable by gDNA sequencing of de novo L1 insertions. The apparent uncoupling of ORF1p and ORF2p expression is striking, and potentially much more pronounced in vivo than in previously characterized experimental systems.

### Example 15

Inhibition of LINE-1

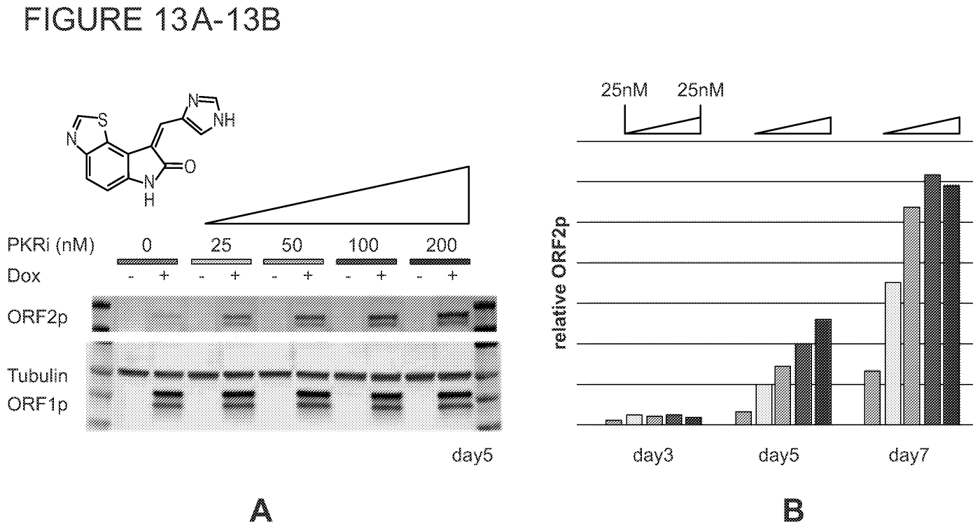

FIG. 13 shows that the experiment shows that treatment with 0.1 uM of a PKR inhibitor increases the accumulation of ORF2p (relative to ORF1p and tubulin) by day 6 of treatment. (Day 1 and 3 show little effect. The effect is sustained at day 10. Higher doses of the drug are toxic.) The finding is significant as proof-of-principle that specific cellular pathways are involved in uncoupling the expression of LINE-1 encoded proteins. As such increasing ORF2p intracellularly in tumors can cause tumor cell death.

The following two articles which include corresponding and related data, results and discussion also are incorporated herein by reference in their entireties: Ardeljan D et al. Mob DNA. 2019 Dec. 31;11:1. doi: 10.1186/s13100-019-0191-2. PMID: 31892958; PMCID: PMC6937734; and Ardeljan D et al., Nat Struct Mol Biol. 2020 Feb.; 27(2):168-178. doi: 10.1038/s41594-020-0372-1. Epub 2020 Feb. 10. PMID: 32042151; PMCID: PMC7080318.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for treatment of a neoplasia in a cell or population of cells comprising:
increasing expression of ORF2p in the cell or population of cells.

2. The method of claim 1 wherein the cell or cells are identified and/or selected for treatment based on an assessed level of ORF1p and/or ORF2p expression.

3. The method of claim 1 wherein the cell or cells are identified and/or selected for treatment based on assessing reduced ORF2p expression as compared to ORF1p expression.

4. The method of claim 1 wherein ORF2p expression is assessed to be less than ORF1p expression.

5. The method of claim 1 wherein one or more PKR targeting agents are administered to the cell or cells.

6. The method of claim 1 wherein a PKR targeting agent is administered to the cell or cells that is selected from 2-aminopurine, 6,8-dihydro-8-(1H-imidazol-5-ylmethylene)-7H-pyrrolo[2,3-g]benzothiazol-7-one, 6-amino-3-methyl-2-oxo-N-phenyl-2,3-dihydro-1H-benzo[d]imidazole-1-carboxamide, and 3-methyl-6-(methylsulphonamido)-2-oxo-N-phenyl-2,3-dihydro-1H-benzo[d]imidazole-1-carboxamide.

7. The method of claim 1 wherein the method comprises contacting the cell or population of cells with a small RNA or SINEUP nucleic acid molecule that affects ORF2p translation. For SINEUP molecules, these might be specific for the inter-ORF spacer near the start codon of open reading frame 2 (ORF2).

8. The method of claim 1 wherein one or more proteasomal inhibitors is administered to the cell or cells.

9. The method of claim 8 wherein the proteasomal inhibitor is selected from the group consisting of: peptide aldehydes, peptide boronates, and nonpeptide inhibitors.

10. The method of claim 8 wherein the proteasomal inhibitor is selected from the group consisting of: Epoxomicin, Lactacystin, Bortezomib, MG-132, Carfilzomib, MLN9708, Ixazomib, PI-1840, ONX-0914, Oprozomib, CEP-18770, and Gabexate Mesylate.

11. A method for treatment of a neoplasia in a subject in need thereof, comprising administering to the subject a biologically active agent which increases expression of ORF2p in the neoplasia of the subject.

12. The method of claim 11 further comprising:
identifying and/or selecting the subject for treatment based on an assessed level of ORF1p and/or ORF2p expression of a suspected neoplasia of the subject; and
administering to the subject a biologically active agent which increases expression of ORF2p in the neoplasia of the subject.

13. The method of claim 11 further comprising:
identifying and/or selecting a subject's neoplasia for treament based on an assessed level of ORF1p and/or ORF2p expression of a suspected neoplasia of the subject; and
administering to the subject a biologically active agent which increases expression of ORF2p in the neoplasia of the subject.

14. The method of claim 11 wherein ORF2p expression is reduced as compared to ORF1p expression.

15. The method of claim 11 wherein the subject and/or neoplasia is selected for treatment based on assessing reduced ORF2p expression as compared to ORF1p expression.

16. The method of claim 11 wherein the biologically active agent is a small RNA or SINEUP nucleic acid molecule that affects ORF2p translation.

17. The method of claim 11 comprising administering to the subject a proteasomal inhibitor.

18. (canceled)

19. The method of claim 17 wherein the proteasomal inhibitor is selected from the group consisting of: Epoxomicin, Lactacystin, Bortezomib, MG-132, Carfilzomib, MLN9708, Ixazomib, PI-1840, ONX-0914, Oprozomib, CEP-18770, and Gabexate Mesylate.

20. The method of claim 1 further comprising administering to the subject one or more additional chemotherapeutic agents.

21. A monoclonal antibody that specifically binds ORF2p antigenic epitopes.

22-25. (canceled)


SEQUENCE LISTING (156 sequences)

SEQ ID NO: 1 - protein, 6 aa, Homo sapiens
DRSTRQ

SEQ ID NO: 2 - protein, 8 aa, Homo sapiens
LHQADLID

SEQ ID NO: 3 - protein, 14 aa, Homo sapiens
KASRRQEITKIRAE

SEQ ID NO: 4 - protein, 9 aa, Homo sapiens
KELEKQEQT

SEQ ID NO: 5 - protein, 8 aa, Homo sapiens
QDIGVGKD

SEQ ID NO: 6 - DNA, 94 bp, Artificial Sequence
  Description of Artificial Sequence Synthetic primer
  modified_base (59)..(62): a, c, t, g, unknown or other
aatgatacggcgaccaccgagatctacactctttccctacacgacgctcttccgatctnnnnaagtagagtcttgtggaaaggacgaaacaccg

SEQ ID NO: 7 - DNA, 95 bp, Artificial Sequence
  Description of Artificial Sequence Synthetic primer
  modified_base (59)..(63): a, c, t, g, unknown or other
aatgatacggcgaccaccgagatctacactctttccctacacgacgctcttccgatctnnnnnacacgatctcttgtggaaaggacgaaacaccg

SEQ ID NO: 8 - DNA, 96 bp, Artificial Sequence
  Description of Artificial Sequence Synthetic primer
  modified_base (59)..(64): a, c, t, g, unknown or other
aatgatacggcgaccaccgagatctacactctttccctacacgacgctcttccgatctnnnnnncgcgcggttcttgtggaaaggacgaaacaccg

SEQ ID NO: 9 - DNA, 97 bp, Artificial Sequence
  Description of Artificial Sequence Synthetic primer
  modified_base (59)..(65): a, c, t, g, unknown or other
aatgatacggcgaccaccgagatctacactctttccctacacgacgctcttccgatctnnnnnnncatgatcgtcttgtggaaaggacgaaacaccg

SEQ ID NO: 10 - DNA, 94 bp, Artificial Sequence
  Description of Artificial Sequence Synthetic primer
  modified_base (59)..(62): a, c, t, g, unknown or other
aatgatacggcgaccaccgagatctacactctttccctacacgacgctcttccgatctnnnncgttaccatcttgtggaaaggacgaaacaccg

SEQ ID NO: 11 - DNA, 95 bp, Artificial Sequence
  Description of Artificial Sequence Synthetic primer
  modified_base (59)..(63): a, c, t, g, unknown or other
aatgatacggcgaccaccgagatctacactctttccctacacgacgctcttccgatctnnnnntccttggttcttgtggaaaggacgaaacaccg

SEQ ID NO: 12 - DNA, 96 bp, Artificial Sequence
  Description of Artificial Sequence Synthetic primer
  modified_base (59)..(64): a, c, t, g, unknown or other
aatgatacggcgaccaccgagatctacactctttccctacacgacgctcttccgatctnnnnnnaacgcatttcttgtggaaaggacgaaacaccg

SEQ ID NO: 13 - DNA, 97 bp, Artificial Sequence
  Description of Artificial Sequence Synthetic primer
  modified_base (59)..(65): a, c, t, g, unknown or other
aatgatacggcgaccaccgagatctacactctttccctacacgacgctcttccgatctnnnnnnnacaggtattcttgtggaaaggacgaaacaccg

SEQ ID NO: 14 - DNA, 94 bp, Artificial Sequence
  Description of Artificial Sequence Synthetic primer
  modified_base (59)..(62): a, c, t, g, unknown or other
aatgatacggcgaccaccgagatctacactctttccctacacgacgctcttccgatctnnnnaggtaaggtcttgtggaaaggacgaaacaccg

SEQ ID NO: 15 - DNA, 95 bp, Artificial Sequence
  Description of Artificial Sequence Synthetic primer
  modified_base (59)..(63): a, c, t, g, unknown or other
aatgatacggcgaccaccgagatctacactctttccctacacgacgctcttccgatctnnnnnaacaatggtcttgtggaaaggacgaaacaccg

SEQ ID NO: 16 - DNA, 96 bp, Artificial Sequence
  Description of Artificial Sequence Synthetic primer
  modified_base (59)..(64): a, c, t, g, unknown or other
aatgatacggcgaccaccgagatctacactctttccctacacgacgctcttccgatctnnnnnnactgtatctcttgtggaaaggacgaaacaccg

SEQ ID NO: 17 - DNA, 97 bp, Artificial Sequence
  Description of Artificial Sequence Synthetic primer
  modified_base (59)..(65): a, c, t, g, unknown or other
aatgatacggcgaccaccgagatctacactctttccctacacgacgctcttccgatctnnnnnnnaggtcgcatcttgtggaaaggacgaaacaccg

SEQ ID NO: 18 - DNA, 96 bp, Artificial Sequence
  Description of Artificial Sequence Synthetic primer
  modified_base (67)..(71): a, c, t, g, unknown or other
caagcagaagacggcatacgagatacacgatcgtgactggagttcagacgtgtgctcttccgatctnnnnntctactattctttcccctgcactgt

SEQ ID NO: 19 - DNA, 50 bp, Artificial Sequence
  Description of Artificial Sequence Synthetic primer
gtaccacttcctaccctcgaaaggcctctgatgggcaagaagcagaaccg

SEQ ID NO: 20 - DNA, 51 bp, Artificial Sequence
  Description of Artificial Sequence Synthetic primer
tcatgtctatcgatggaagcttggcctgacatcagttgccgccgatcaggc

SEQ ID NO: 21 - DNA, 20 bp, Artificial Sequence
  Description of Artificial Sequence Synthetic primer
tgtcttgtacccttgtgcct

SEQ ID NO: 22 - DNA, 20 bp, Artificial Sequence
  Description of Artificial Sequence Synthetic primer
aagatgtagagcgggccttt

SEQ ID NO: 23 - DNA, 18 bp, Artificial Sequence
  Description of Artificial Sequence Synthetic primer
ggatttggtcgtattggg

SEQ ID NO: 24 - DNA, 19 bp, Artificial Sequence
  Description of Artificial Sequence Synthetic primer
ggaagatggtgatgggatt

SEQ ID NO: 25 - DNA, 21 bp, Artificial Sequence
  Description of Artificial Sequence Synthetic primer
agctgcttaatctcctcaggg

SEQ ID NO: 26 - DNA, 20 bp, Artificial Sequence
  Description of Artificial Sequence Synthetic primer
tctcctgttgtgcttctcca

SEQ ID NO: 27 - DNA, 23 bp, Artificial Sequence
  Description of Artificial Sequence Synthetic primer
tgtctcataatggcttgtcatct

SEQ ID NO: 28 - DNA, 23 bp, Artificial Sequence
  Description of Artificial Sequence Synthetic primer
ggccaaataatcttccaattgcg

SEQ ID NO: 29 - DNA, 20 bp, Artificial Sequence
  Description of Artificial Sequence Synthetic primer
aaacaactgctccatctgcg

SEQ ID NO: 30 - DNA, 20 bp, Artificial Sequence
  Description of Artificial Sequence Synthetic primer
tgcaaagcctcagaatctgc

SEQ ID NO: 31 - DNA, 20 bp, Artificial Sequence
  Description of Artificial Sequence Synthetic primer
gctttcaaatcccttccgct

SEQ ID NO: 32 - DNA, 20 bp, Artificial Sequence
  Description of Artificial Sequence Synthetic primer
taggcagagatcgcataccc

SEQ ID NO: 33 - DNA, 20 bp, Artificial Sequence
  Description of Artificial Sequence Synthetic primer
aagatcatccgcgccatcta

SEQ ID NO: 34 - DNA, 20 bp, Artificial Sequence
  Description of Artificial Sequence Synthetic primer
tcagcttcacctcctccttg

SEQ ID NO: 35 - DNA, 20 bp, Artificial Sequence
  Description of Artificial Sequence Synthetic primer
ccattcacaatcccactaca

SEQ ID NO: 36 - DNA, 20 bp, Artificial Sequence
  Description of Artificial Sequence Synthetic primer
tgagcattcgtagcccagca

SEQ ID NO: 37 - DNA, 20 bp, Artificial Sequence
  Description of Artificial Sequence Synthetic primer
gatccactcacagtttccat

SEQ ID NO: 38 - DNA, 20 bp, Artificial Sequence
  Description of Artificial Sequence Synthetic primer
ggtgccctatgagccgcctg

SEQ ID NO: 39 - DNA, 20 bp, Artificial Sequence
  Description of Artificial Sequence Synthetic primer
gtcaccgagacaccactgga

SEQ ID NO: 40 - DNA, 20 bp, Artificial Sequence
  Description of Artificial Sequence Synthetic primer
agtcgaagttccatcgctca

SEQ ID NO: 41 - DNA, 20 bp, Artificial Sequence
  Description of Artificial Sequence Synthetic primer
tgacggtggttacaacacgc

SEQ ID NO: 42 - DNA, 20 bp, Artificial Sequence
  Description of Artificial Sequence Synthetic primer
gacacacagaaccttccgag

SEQ ID NO: 43 - DNA, 20 bp, Artificial Sequence
  Description of Artificial Sequence Synthetic primer
cgagatgaatccctcataca

SEQ ID NO: 44 - DNA, 20 bp, Artificial Sequence
  Description of Artificial Sequence Synthetic primer
tatgactgtattcttatacc

SEQ ID NO: 45 - DNA, 20 bp, Artificial Sequence
  Description of Artificial Sequence Synthetic primer
agttgactgacaatgagtcg

SEQ ID NO: 46 - DNA, 20 bp, Artificial Sequence
  Description of Artificial Sequence Synthetic primer
tcctaggaaaaacccttctg

SEQ ID NO: 47 - DNA, 20 bp, Artificial Sequence
  Description of Artificial Sequence Synthetic primer
aggtgttagacaaacccagt

SEQ ID NO: 48 - DNA, 20 bp, Artificial Sequence
  Description of Artificial Sequence Synthetic primer
ggaaaacgaaataactcaag

SEQ ID NO: 49 - DNA, 20 bp, Artificial Sequence
  Description of Artificial Sequence Synthetic primer
atacatcggatgatgatacc

SEQ ID NO: 50 - DNA, 20 bp, Artificial Sequence
  Description of Artificial Sequence Synthetic primer
acattagaagtacgcctagg

SEQ ID NO: 51 - DNA, 20 bp, Artificial Sequence
  Description of Artificial Sequence Synthetic primer
aaggaaagagtctactgtcg

SEQ ID NO: 52 - DNA, 20 bp, Artificial Sequence
  Description of Artificial Sequence Synthetic primer
tgacacggtcctccgcatcg

SEQ ID NO: 53 - DNA, 20 bp, Artificial Sequence
  Description of Artificial Sequence Synthetic primer
ttagctcagaagtgcacagg

SEQ ID NO: 54 - DNA, 20 bp, Artificial Sequence
  Description of Artificial Sequence Synthetic primer
cttggacaagttctaccgga

SEQ ID NO: 55 - DNA, 20 bp, Artificial Sequence
  Description of Artificial Sequence Synthetic primer
ttggtgaccatctcagaact

SEQ ID NO: 56 - DNA, 20 bp, Artificial Sequence
  Description of Artificial Sequence Synthetic primer
ccccaaccagcaccagactg

SEQ ID NO: 57 - DNA, 20 bp, Artificial Sequence
  Description of Artificial Sequence Synthetic primer
aagacccccccaaagatcct

SEQ ID NO: 58 - DNA, 20 bp, Artificial Sequence
  Description of Artificial Sequence Synthetic primer
aggatcgacttgtggaacaa

SEQ ID NO: 59 - DNA, 20 bp, Artificial Sequence
  Description of Artificial Sequence Synthetic primer
agatatcacacattacagtg

SEQ ID NO: 60 - DNA, 20 bp, Artificial Sequence
  Description of Artificial Sequence Synthetic primer
cattcagaaagggctcgtaa

SEQ ID NO: 61 - DNA, 20 bp, Artificial Sequence
  Description of Artificial Sequence Synthetic primer
tgactttgggtaccatcatg

SEQ ID NO: 62 - DNA, 20 bp, Artificial Sequence
  Description of Artificial Sequence Synthetic primer
cctgccatatcagtcagccg

SEQ ID NO: 63 - DNA, 20 bp, Artificial Sequence
  Description of Artificial Sequence Synthetic primer
cagagtgagccgtttgtccg

SEQ ID NO: 64 - DNA, 20 bp, Artificial Sequence
  Description of Artificial Sequence Synthetic primer
tgcagccgtagtcttgattg

SEQ ID NO: 65 - DNA, 20 bp, Artificial Sequence
  Description of Artificial Sequence Synthetic primer
tgtgccggaagtgccgcaca

SEQ ID NO: 66 - DNA, 20 bp, Artificial Sequence
  Description of Artificial Sequence Synthetic primer
ctacgacagtcacttcgatg

SEQ ID NO: 67 - DNA, 20 bp, Artificial Sequence
  Description of Artificial Sequence Synthetic primer
ctaaccgttagagcttccga

SEQ ID NO: 68 - DNA, 20 bp, Artificial Sequence
  Description of Artificial Sequence Synthetic primer
agggcctttatacgccacca

SEQ ID NO: 69 - DNA, 20 bp, Artificial Sequence
  Description of Artificial Sequence Synthetic primer
ttctggactggtgttatggg

SEQ ID NO: 70 - DNA, 20 bp, Artificial Sequence
  Description of Artificial Sequence Synthetic primer
attaccagatcgtcattacc

SEQ ID NO: 71 - DNA, 20 bp, Artificial Sequence
  Description of Artificial Sequence Synthetic primer
gagcaaactttgcagatcca

SEQ ID NO: 72 - DNA, 20 bp, Artificial Sequence
  Description of Artificial Sequence Synthetic primer
aaaatgggcaacgagaaacc

SEQ ID NO: 73 - DNA, 20 bp, Artificial Sequence
  Description of Artificial Sequence Synthetic primer
gaagagcaggaatcttcaga

SEQ ID NO: 74 - DNA, 20 bp, Artificial Sequence
  Description of Artificial Sequence Synthetic primer
tcctcccatcacaaaaaggt

SEQ ID NO: 75 - DNA, 20 bp, Artificial Sequence
  Description of Artificial Sequence Synthetic primer
caattgaatctgttattgca

SEQ ID NO: 76 - DNA, 20 bp, Artificial Sequence
  Description of Artificial Sequence Synthetic primer
cttacctggtcaggtcaagg

SEQ ID NO: 77 - DNA, 20 bp, Artificial Sequence
  Description of Artificial Sequence Synthetic primer
agctatcctgcgctacagtg

SEQ ID NO: 78 - DNA, 20 bp, Artificial Sequence
  Description of Artificial Sequence Synthetic primer
tatggtttccaatatcgaca

SEQ ID NO: 79 - DNA, 20 bp, Artificial Sequence
  Description of Artificial Sequence Synthetic primer
ggatcatacgattccctcag

SEQ ID NO: 80 - DNA, 20 bp, Artificial Sequence
  Description of Artificial Sequence Synthetic primer
aaaacaccagacgaattggc

SEQ ID NO: 81 - DNA, 20 bp, Artificial Sequence
  Description of Artificial Sequence Synthetic primer
atttgcatccaaatatactg

SEQ ID NO: 82 - DNA, 20 bp, Artificial Sequence
  Description of Artificial Sequence Synthetic primer
aaagagcacttggtaatcgg

SEQ ID NO: 83 - DNA, 20 bp, Artificial Sequence
  Description of Artificial Sequence Synthetic primer
tgtagcgtgggtaaatacga

SEQ ID NO: 84 - DNA, 20 bp, Artificial Sequence
  Description of Artificial Sequence Synthetic primer
gacgacaagccagacgactc

SEQ ID NO: 85 - DNA, 20 bp, Artificial Sequence
  Description of Artificial Sequence Synthetic primer
ccaggaggcttaggaatttg

SEQ ID NO: 86 - DNA, 20 bp, Artificial Sequence
  Description of Artificial Sequence Synthetic primer
aggtggttcagctcgcatag

SEQ ID NO: 87 - DNA, 20 bp, Artificial Sequence
  Description of Artificial Sequence Synthetic primer
gtccggtagtgcccgaaatg

SEQ ID NO: 88 - DNA, 20 bp, Artificial Sequence
  Description of Artificial Sequence Synthetic primer
aaggagcgtctatgcaacca

SEQ ID NO: 89 - DNA, 20 bp, Artificial Sequence
  Description of Artificial Sequence Synthetic primer
gtttgcctacctcgtgaacg

SEQ ID NO: 90 - DNA, 20 bp, Artificial Sequence
  Description of Artificial Sequence Synthetic primer
accaacagccgggtcacggt

SEQ ID NO: 91 - DNA, 20 bp, Artificial Sequence
  Description of Artificial Sequence Synthetic primer
tcgacagagacaatctcgca

SEQ ID NO: 92 - DNA, 20 bp, Artificial Sequence
  Description of Artificial Sequence Synthetic primer
gggcgaggacctgaacgact

SEQ ID NO: 93 - DNA, 20 bp, Artificial Sequence
  Description of Artificial Sequence Synthetic primer
cgaggcgcagatcaacaacg

SEQ ID NO: 94 - DNA, 20 bp, Artificial Sequence
  Description of Artificial Sequence Synthetic primer
tggtggtaaatgtaacgtgg

SEQ ID NO: 95 - DNA, 20 bp, Artificial Sequence
  Description of Artificial Sequence Synthetic primer
ctctacaagtcgaactttgg

SEQ ID NO: 96 - DNA, 23 bp, Artificial Sequence
  Description of Artificial Sequence Synthetic primer
tgagcggctacaagatcaacgtg

SEQ ID NO: 97 - DNA, 40 bp, Artificial Sequence
  Description of Artificial Sequence Synthetic primer
gctcatgaagtccttgcccatgccgatgtcctggatggtg

SEQ ID NO: 98 - DNA, 40 bp, Artificial Sequence
  Description of Artificial Sequence Synthetic primer
caccatccaggacatcggcatgggcaaggacttcatgagc

SEQ ID NO: 99 - DNA, 21 bp, Artificial Sequence
  Description of Artificial Sequence Synthetic primer
acatgtgcacattgtgcaggt

SEQ ID NO: 100 - protein, 10 aa, Homo sapiens
MENDFDELRE

SEQ ID NO: 101 - DNA, 26 bp, Artificial Sequence
  Description of Artificial Sequence Synthetic primer
agatatacctaatgctagatgacaca

SEQ ID NO: 102 - protein, 6 aa, Artificial Sequence
  Description of Artificial Sequence Synthetic 6xHis tag
HHHHHH

SEQ ID NO: 103 - protein, 8 aa, Homo sapiens
AAIINMFK

SEQ ID NO: 104 - protein, 14 aa, Homo sapiens
ADIQIQEIQRTPQR

SEQ ID NO: 105 - protein, 14 aa, Homo sapiens
ANIQIQEIQRTPQR

SEQ ID NO: 106 - protein, 14 aa, Homo sapiens
ANVQIQEIQRTSQR

SEQ ID NO: 107 - protein, 10 aa, Homo sapiens
DFVTTRPALK

SEQ ID NO: 108 - protein, 14 aa, Homo sapiens
DFVTTRPALQELLK

SEQ ID NO: 109 - protein, 7 aa, Homo sapiens
EALNMER

SEQ ID NO: 110 - protein, 7 aa, Homo sapiens
EKNFQPR

SEQ ID NO: 111 - protein, 7 aa, Homo sapiens
ELREECR

SEQ ID NO: 112 - protein, 10 aa, Homo sapiens
EWGPIFNILK

SEQ ID NO: 113 - protein, 8 aa, Homo sapiens
FVSEGEIK

SEQ ID NO: 114 - protein, 12 aa, Homo sapiens
IQIQEIQRMPQR

SEQ ID NO: 115 - protein, 18 aa, Homo sapiens
LENTLQDIIQENFPNLAR

SEQ ID NO: 116 - protein, 14 aa, Homo sapiens
LIGVPESDAENGTK

SEQ ID NO: 117 - protein, 14 aa, Homo sapiens
LIGVPESDGENGTK

SEQ ID NO: 118 - protein, 14 aa, Homo sapiens
LIGVPESDVENGTK

SEQ ID NO: 119 - protein, 14 aa, Homo sapiens
LIGVPQSDVENGTK

SEQ ID NO: 120 - protein, 10 aa, Homo sapiens
LSFISEGEIK

SEQ ID NO: 121 - protein, 10 aa, Homo sapiens
LSFVSEGEIK

SEQ ID NO: 122 - protein, 13 aa, Homo sapiens
LTADLSAETLQAR

SEQ ID NO: 123 - protein, 13 aa, Homo sapiens
NEESLQEIWDYVK

SEQ ID NO: 124 - protein, 13 aa, Homo sapiens
NEQSLQEIWDYMK

SEQ ID NO: 125 - protein, 13 aa, Homo sapiens
NEQSLQEIWDYVK

SEQ ID NO: 126 - protein, 8 aa, Homo sapiens
NLEECITR

SEQ ID NO: 127 - protein, 13 aa, Homo sapiens
NQQSLQEIWDYVK

SEQ ID NO: 128 - protein, 11 aa, Homo sapiens
NQYQPLQNHAK

SEQ ID NO: 129 - protein, 9 aa, Homo sapiens
NVQIQEIQR

SEQ ID NO: 130 - protein, 7 aa, Homo sapiens
PIYNILK

SEQ ID NO: 131 - protein, 11 aa, Homo sapiens
QANIQIQEIQR

SEQ ID NO: 132 - protein, 11 aa, Homo sapiens
QANVQIQEIQR

SEQ ID NO: 133 - protein, 11 aa, Homo sapiens
QANVQIQETQR

SEQ ID NO: 134 - protein, 11 aa, Homo sapiens
QANVQIQEVQR

SEQ ID NO: 135 - protein, 14 aa, Homo sapiens
QARREWGPIFNIVK

SEQ ID NO: 136 - protein, 8 aa, Homo sapiens
QSQLLEMK

SEQ ID NO: 137 - protein, 11 aa, Homo sapiens
REWGPIFNILK

SEQ ID NO: 138 - protein, 14 aa, Homo sapiens
RNEQSLQEIWDYVK

SEQ ID NO: 139 - protein, 9 aa, Homo sapiens
SFISEGEIK

SEQ ID NO: 140 - protein, 13 aa, Homo sapiens
SNYSELQEDIQTK

SEQ ID NO: 141 - protein, 7 aa, Homo sapiens
SNYSELR

SEQ ID NO: 142 - protein, 12 aa, Homo sapiens
SNYSELREDIQT

SEQ ID NO: 143 - protein, 13 aa, Homo sapiens
SNYSELREDIQTK

SEQ ID NO: 144 - protein, 9 aa, Homo sapiens
SRCDQLEER

SEQ ID NO: 145 - protein, 9 aa, Homo sapiens
SRCDQLEGR

SEQ ID NO: 146 - protein, 24 aa, Homo sapiens
SSSPAMEQTWMENDFDELREEGFR

SEQ ID NO: 147 - protein, 11 aa, Homo sapiens
TIQENFPNLAR

SEQ ID NO: 148 - protein, 10 aa, Homo sapiens
TQENFPNLAR

SEQ ID NO: 149 - protein, 15 aa, Homo sapiens
VRVTADLSAETLQAR

SEQ ID NO: 150 - protein, 12 aa, Homo sapiens
VSAMEDEMNEMK

SEQ ID NO: 151 - protein, 13 aa, Homo sapiens
VSAMEDEMNEMKR

SEQ ID NO: 152 - protein, 13 aa, Homo sapiens
VSAMEDEMNEVKR

SEQ ID NO: 153 - protein, 9 aa, Homo sapiens
YQLLQNHAK

SEQ ID NO: 154 - protein, 9 aa, Homo sapiens
YQPLQNHAK

SEQ ID NO: 155 - protein, 9 aa, Homo sapiens
YQPLQNHDK

SEQ ID NO: 156 - protein, 7 aa, Homo sapiens
NQIDTIK